SEQUENCE LISTING (78 sequences)

SEQ ID NO: 1 - protein, 123 aa, Synechocystis sp. PCC6803
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 2 - protein, 37 aa, Synechocystis sp. PCC6803
MVKVIGRRSLGVQRIFDIGLPQDHNFLLANGAIAANC

SEQ ID NO: 3 - DNA, 75 bp, Artificial Sequence
  Modified from Synechocystis sp. PCC6803, to contain plant preferred codons
tgcctttctttcggaactgagatccttaccgttgagtacggaccacttcctattggtaagatcgtttctgaggaa

SEQ ID NO: 4 - DNA, 75 bp, Artificial Sequence
  Modified from Synechocystis sp. PCC6803, to contain plant preferred codons
attaactgctcagtgtactctgttgatccagaaggaagagtttacactcaggctatcgcacaatggcacgatagg

SEQ ID NO: 5 - DNA, 75 bp, Artificial Sequence
  Modified from Synechocystis sp. PCC6803, to contain plant preferred codons
ggtgaacaagaggttctcgagtacgagcttgaagatggatccgttattcgtgctacctctgaccatagattcttg

SEQ ID NO: 6 - DNA, 75 bp, Artificial Sequence
  Modified from Synechocystis sp. PCC6803, to contain plant preferred codons
actacagattatcagcttctcgctatcgaggaaatctttgctaggcaacttgatctccttactttggagaacatc

SEQ ID NO: 7 - DNA, 69 bp, Artificial Sequence
  Modified from Synechocystis sp. PCC6803, to contain plant preferred codons
aagcagacagaagaggctcttgacaaccacagacttccattccctttgctcgatgctggaaccatcaag

SEQ ID NO: 8 - DNA, 30 bp, Artificial Sequence
  Modified from Synechocystis sp. PCC6803, to contain plant preferred codons
cttgatggttccagcatcgagcaaagggaa

SEQ ID NO: 9 - DNA, 75 bp, Artificial Sequence
  Modified from Synechocystis sp. PCC6803, to contain plant preferred codons
tggaagtctgtggttgtcaagagcctcttctgtctgcttgatgttctccaaagtaaggagatcaagttgcctagc

SEQ ID NO: 10 - DNA, 75 bp, Artificial Sequence
  Modified from Synechocystis sp. PCC6803, to contain plant preferred codons
aaagatttcctcgatagcgagaagctgataatctgtagtcaagaatctatggtcagaggtagcacgaataacgga

SEQ ID NO: 11 - DNA, 75 bp, Artificial Sequence
  Modified from Synechocystis sp. PCC6803, to contain plant preferred codons
tccatcttcaagctcgtactcgagaacctcttgttcacccctatcgtgccattgtgcgatagcctgagtgtaaac

SEQ ID NO: 12 - DNA, 75 bp, Artificial Sequence
  Modified from Synechocystis sp. PCC6803, to contain plant preferred codons
tcttccttctggatcaacagagtacactgagcagttaatttcctcagaaacgatcttaccaataggaagtggtcc

SEQ ID NO: 13 - DNA, 39 bp, Artificial Sequence
  Modified from Synechocystis sp. PCC6803, to contain plant preferred codons
gtactcaacggtaaggatctcagttccgaaagaaaggca

SEQ ID NO: 14 - DNA, 75 bp, Artificial Sequence
  Modified from Synechocystis sp. PCC6803, to contain plant preferred codons
atggttaaggtgattggaagacgttctcttggtgttcaaaggatcttcgatatcggattgccacaagaccacaac

SEQ ID NO: 15 - DNA, 36 bp, Artificial Sequence
  Modified from Synechocystis sp. PCC6803, to contain plant preferred codons
tttcttctcgctaatggtgccatcgctgccaattgc

SEQ ID NO: 16 - DNA, 75 bp, Artificial Sequence
  Modified from Synechocystis sp. PCC6803, to contain plant preferred codons
gcaattggcagcgatggcaccattagcgagaagaaagttgtggtcttgtggcaatccgatatcgaagatcctttg

SEQ ID NO: 17 - DNA, 36 bp, Artificial Sequence
  Modified from Synechocystis sp. PCC6803, to contain plant preferred codons
aacaccaagagaacgtcttccaatcaccttaaccat

SEQ ID NO: 18 - DNA, 21 bp, Artificial Sequence
  Modified from Synechocystis sp. PCC6803, to contain plant preferred codons
tgcctttctttcggaactgag

SEQ ID NO: 19 - DNA, 24 bp, Artificial Sequence
  Modified from Synechocystis sp. PCC6803, to contain plant preferred codons
tcacttgatggttccagcatcgag

SEQ ID NO: 20 - DNA, 24 bp, Artificial Sequence
  Modified from Synechocystis sp. PCC6803, to contain plant preferred codons
ccatggttaaggtgattggaagac

SEQ ID NO: 21 - DNA, 21 bp, Artificial Sequence
  Modified from Synechocystis sp. PCC6803, to contain plant preferred codons
gcaattggcagcgatggcacc

SEQ ID NO: 22 - DNA, 369 bp, Artificial Sequence
  Modified from Synechocystis sp. PCC6803 SspE, to contain plant preferred codons
tgcctttctttcggaactgagatccttaccgttgagtacggaccacttcctattggtaagatcgtttctgaggaaattaactgctcagtgtactctgttgatccagaaggaagagtttacactcaggctatcgcacaatggcacgataggggtgaacaagaggttctcgagtacgagcttgaagatggatccgttattcgtgctacctctgaccatagattcttgactacagattatcagcttctcgctatcgaggaaatttttgctaggcaacttgatctccttactttggagaacattaagcagacagaagaggctcttgacaaccacagacttccattccctttgctcgatgctggaaccatcaag

SEQ ID NO: 23 - protein, 123 aa, Artificial Sequence; identical to SEQ ID NO: 1
  Synthetic Construct

SEQ ID NO: 24 - DNA, 111 bp, Artificial Sequence
  Modified from Synechocystis sp. PCC6803 SspE, to contain plant preferred codons
atggttaaggtgattggaagacgttctcttggtgttcaaaggatcttcgatatcggattgccacaagaccacaactttcttctcgctaatggtgccatcgctgcaaattgc

SEQ ID NO: 25 - protein, 37 aa, Artificial Sequence; identical to SEQ ID NO: 2
  Synthetic Construct

SEQ ID NO: 26 - DNA, 48 bp, Artificial Sequence
  Primer to introduce CDS for peptide MAHHHHHH at the N-terminus of GUS
atggctcatcatcatcatcatcatgtacgtcctgtagaaaccccaacc

SEQ ID NO: 27 - DNA, 27 bp, Artificial Sequence
  Primer to introduce a BamHI site after the stop codon of GUS
ggatccttgtttgcctccctgctgcgg

SEQ ID NO: 28 - protein, 618 aa, Artificial Sequence
  Modified GUS protein, with 6x His tags on the N-terminus and C-terminus
MAHHHHHHVRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQGSHHHHHH

SEQ ID NO: 29 - DNA, 21 bp, Artificial Sequence
  Primer to amplify GUS
cgcagcgtaatgctctacacc

SEQ ID NO: 30 - DNA, 21 bp, Artificial Sequence
  Primer to amplify GUS
ccgtaataacggttcaggcac

SEQ ID NO: 31 - DNA, 45 bp, Artificial Sequence
  Primer to amplify the 2 um yeast replication origin and a Trp selective marker
agggaacaaaagctggagctccaccagagggccaagagggagggc

SEQ ID NO: 32 - DNA, 45 bp, Artificial Sequence
  Primer to amplify the 2 um yeast replication origin and a Trp selective marker
cactagttctagagcggccgccaccatatgatccaatatcaaagg

SEQ ID NO: 33 - DNA, 45 bp, Artificial Sequence
  Primer for PCR-directed recombination for in-frame fusion of GUS-n/ Int-n and Int-c/GUS-c
ggatctcagttccgaaagaaaggcagtcttgcgcgacatgcgtca

SEQ ID NO: 34 - DNA, 45 bp, Artificial Sequence
  Primer for PCR-directed recombination for in-frame fusion of GUS-n/ Int-n and Int-c/GUS-c
cccctcgaggtcgacggtatcgatatccatggctcatcatcatca

SEQ ID NO: 35 - DNA, 45 bp, Artificial Sequence
  Primer for PCR-directed recombination for in-frame fusion of GUS-n/ Int-n and Int-c/GUS-c
gtccgtactcaacggtaaggatctcgtcttgcgcgacatgcgtca

SEQ ID NO: 36 - DNA, 45 bp, Artificial Sequence
  Primer for PCR-directed recombination for in-frame fusion of GUS-n/ Int-n and Int-c/GUS-c
cgctaatggtgccatcgctgccaattgtaaccacgcgtctgttga

SEQ ID NO: 37 - DNA, 22 bp, Artificial Sequence
  Primer for PCR-directed recombination for in-frame fusion of GUS-n/ Int-n and Int-c/GUS-c
cgaggtcgacggtatcgataag

SEQ ID NO: 38 - protein, 326 aa, Artificial Sequence
  GUSn/Intn fusion
MAHHHHHHVRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 39 - protein, 320 aa, Artificial Sequence
  GUSn/Intn(6) fusion, with 6 amino acid deletion in Intn
MAHHHHHHVRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 40 - protein, 450 aa, Artificial Sequence
  Intc/GUSc fusion
MVKVIGRRSLGVQRIFDIGLPQDHNFLLANGAIAANCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQGSHHHHHH

SEQ ID NO: 41 - DNA, 41 bp, Artificial Sequence
  Primer to amplify NOS terminator region
gcgtcgacagtcactctagagacatcgatctagtaacatag

SEQ ID NO: 42 - DNA, 41 bp, Artificial Sequence
  Primer to amplify NOS terminator region
ggggtaccccatgcggccgcctaaagaaggagtgcgtcgaa

SEQ ID NO: 43 - DNA, 18 bp, Artificial Sequence
  18 bp polylinker
ggtacccgatccaattcc

SEQ ID NO: 44 - DNA, 26 bp, Artificial Sequence
  Primer
gaccatggccaatttactgaccgtac

SEQ ID NO: 45 - DNA, 27 bp, Artificial Sequence
  Primer
cgaaagaaaggcagcagcgatcgctat

SEQ ID NO: 46 - DNA, 29 bp, Artificial Sequence
  Primer
atagcgatcgctgctgcctttctttcgga

SEQ ID NO: 47 - DNA, 27 bp, Artificial Sequence
  Primer
atgtcgactcacttgatggttccagca

SEQ ID NO: 48 - DNA, 3034 bp, Artificial Sequence
  Sequence of 3034 bp Asp 718 fragment containing 35S-CreN-IntN-3′ocs gene in plasmid pGV947
ggtacccgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatggccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactagttatgcggcggatccgaaaagaaaacgttgatgccggtgaacgtgcaaaacaggctctagcgttcgaacgcactgatttcgaccaggttcgttcactcatggaaaatagcgatcgctgctgcctttctttcggaactgagatccttaccgttgagtacggaccacttcctattggtaagatcgtttctgaggaaattaactgctcagtgtactctgttgatccagaaggaagagtttacactcaggctatcgcacaatggcacgataggggtgaacaagaggttctcgagtacgagcttgaagatggatccgttattcgtgctacctctgaccatagattcttgactacagattatcagcttctcgctatcgaggaaatctttgctaggcaacttgatctccttactttggagaacatcaagcagacagaagaggctcttgacaaccacagacttccattccctttgctcgatgctggaaccatcaagtgagtcgacataatcactagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttatcgataccgtcgacctcgagggggggcccggtacc

SEQ ID NO: 49 - DNA, 17 bp, Artificial Sequence
  17 bp linker sequence
gtcgacataatcactag

SEQ ID NO: 50 - DNA, 60 bp, Artificial Sequence
  60 bp polylinker
caggacctgcaggcatgcaagcttatcgataccgtcgacctcgagggggggcccggtacc

SEQ ID NO: 51 - DNA, 27 bp, Artificial Sequence
  Primer
gaccatggttaaggtgattggaagacg

SEQ ID NO: 52 - DNA, 30 bp, Artificial Sequence
  Primer
tacgtatatcctggcaattggcagcgatgg

SEQ ID NO: 53 - DNA, 31 bp, Artificial Sequence
  Primer
cgctgccaattgccaggatatacgtaatctg

SEQ ID NO: 54 - DNA, 28 bp, Artificial Sequence
  Primer
agtcgacctaatcgccatcttccagcag

SEQ ID NO: 55 - DNA, 2873 bp, Artificial Sequence
  Sequence of 2873 bp Asp718 fragment containing 35SIntC-CreC3′ocs in plasmid pGV951
ggtacccgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatggttaaggtgattggaagacgttctcttggtgttcaaaggatcttcgatatcggattgccacaagaccacaactttcttctcgctaatggtgccatcgctgccaattgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggtcgactatcactagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttatcgataccgtcgacctcgagggggggcccggtacc

SEQ ID NO: 56 - DNA, 14 bp, Artificial Sequence
  15 bp linker
tcgactatcactag

SEQ ID NO: 57 - DNA, 5449 bp, Artificial Sequence
  Sequence of 5449 bp Sal I-HindIII fragment containing the blocked GUS reporter gene for Cre-Lox excision in plasmid pGV801
gtcgactctagaggatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgataacttcgtatagcatacattatacgaagttatcctaggatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcctaggtaaataacttcgtatagcatacattatacgaagttattagtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtggggaattccccgggggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgactctagaaagctt

SEQ ID NO: 58 - DNA, 24 bp, Artificial Sequence
  24 bp polylinker
gtcgactctagaggatccaattcc

SEQ ID NO: 59 - DNA, 34 bp, Artificial Sequence
  Lox P sequence
ataacttcgtatagcatacattatacgaagttat

SEQ ID NO: 60 - DNA, 22 bp, Artificial Sequence
  22 bp polylinker
tggggaattccccgggggtacc

SEQ ID NO: 61 - DNA, 18 bp, Artificial Sequence
  18 bp polylinker
gtcgactctagaaagctt

SEQ ID NO: 62 - protein, 605 aa, Artificial Sequence
  Elastin-based protein polymer (Zhang et al., Plant Cell Rep. 16(3-4)174-179 (1996))
GVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVP

SEQ ID NO: 63 - protein, 8 aa, Artificial Sequence
  Coding sequence introduced by oligomer HGUSH-n
MAHHHHHH

SEQ ID NO: 64 - protein, 13 aa, Artificial Sequence
  Insertion sequence in pGY101 (a pBluscript-based plasmid)
MARSRGSHHHHHH

SEQ ID NO: 65 - protein, 6 aa, Artificial Sequence
  Residues deleted from IntN to create GUSn/Intn(6) fusion
CLSFGT

SEQ ID NO: 66 - protein, 12 aa, Artificial Sequence
  12-amino acid N-terminal amino acid extension to GUS ORF
ITSYSIHYTKLL

SEQ ID NO: 67 - DNA, 21 bp, artificial sequence
  primer
gtatcaccgtttgtgtgaaca

SEQ ID NO: 68 - DNA, 32 bp, artificial sequence
  primer
ggaattcctcagatgttctccaaagtaaggag

SEQ ID NO: 69 - DNA, 32 bp, artificial sequence
  primer
ggaattcctcaatctgtagtcaagaatctatg

SEQ ID NO: 70 - DNA, 32 bp, artificial sequence
  primer
ggaattcctcaaacctcttgttcacccctatc

SEQ ID NO: 71 - protein, 78 aa, Synechocystis sp. PCC6803
CLSFGTEILTVEYGPLPIGKIVSEEINCSVYSVDPEGRVYTQAIAQWHDRGEQEVLEYELEDGSVIRATSDHRFLTTD

SEQ ID NO: 72 - DNA, 234 bp, artificial sequence
  plant codon optimized IntN encoding sequence
tgcctttctttcggaactgagatccttaccgttgagtacggaccacttcctattggtaagatcgtttctgaggaaattaactgctcagtgtactctgttgatccagaaggaagagtttacactcaggctatcgcacaatggcacgataggggtgaacaagaggttctcgagtacgagcttgaagatggatccgttattcgtgctacctctgaccatagattcttgactacagat

SEQ ID NO: 73 - protein, 12 aa, Synechocystis sp. PCC6803
CLSFGTEILTVE

SEQ ID NO: 74 - protein, 15 aa, Synechocystis sp. PCC6803
DGSVIRATSDHRFLT

SEQ ID NO: 75 - protein, 13 aa, Trichodesmium erythraeum
CLTYETEIMTVEY

SEQ ID NO: 76 - protein, 15 aa, Trichodesmium erythraeum
DGTVIRATPEHKFMT

SEQ ID NO: 77 - protein, 12 aa, artificial
  compiled sequence
CLXXXTEIXTVE

SEQ ID NO: 78 - protein, 15 aa, artificial
  compiled sequence
DGXVIRATXXHXFXT

(19) United States
(12) Patent Application Publication
(10) Pub. No.: US 2004/0172688 A1
(43) Pub. Date: Sep. 2, 2004

(54) Intein-mediated protein splicing

(76) Inventors: Narendra S. Yadav, Chadds Ford, PA (US); Jianjun Yang, Hockessin, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/799,326

(22) Filed: Mar. 12, 2004

Related U.S. Application Data
(63) Continuation-in-part of application No. 10/356,088, filed on Jan. 31, 2003.
(60) Provisional application No. 60/354,395, filed on Feb. 4, 2002.

(51) Int. Cl. A01H 1/00; C12N 15/82; C12N 5/04

(52) U.S. Cl. 800/288; 435/69.1; 435/320.1; 435/419

(57) ABSTRACT

The present invention provides methods for intein-mediated protein splicing, particularly in plants. This permits in vivo and in vitro synthesis of homogeneous and large multi-functional hybrid protein polymers and circular proteins. Additionally, methods are provided which are suitable for the regulation of transgene expression, such that a particular transgene is expressed only under selected environmental conditions, in selected plant tissues, at selected development stages, or in selected plant generations.

This application is a Continuation-in-Part of U.S. Ser. No. 10/356088, filed on Jan. 31, 2003 now pending, which claims the benefit of United States Provisional Application 60/354395, filed Feb. 4, 2002, now expired.

### FIELD OF INVENTION

The invention relates to the field of molecular biology and plant genetics. More specifically, this invention describes a technique to produce proteins in transgenic plants using intein-mediated protein splicing technology.

### BACKGROUND OF THE INVENTION

Inteins (Internal protein fragments) are in-frame intervening sequences that disrupt the coding region of a host gene. These internal protein elements mediate the post-translational protein splicing process, catalyzing a series of reactions to remove the intein from the protein precursor and to ligate the flanking external protein fragments, known as exteins, into a mature protein (Perler, F. B. Cell 92:1-4 (1998)). A typical intein element consists of 400 to 500 amino acid residues and contains four conserved protein splicing motifs (A, B, F, and G) which are separated by a homing endonuclease coding region. The endonuclease does not play a role in protein splicing and can be deleted from the intein sequence without impacting the intein's function (Chong, S. and Xu, M.-Q. J. Biol. Chem. 272:15587-15590 (1997); Shingledecker, K. et al. Gene. 207:187-195 (1998)). A few mini-inteins have been identified, which do not contain a homing endonuclease; these are approximately 150 amino acids in size (Perler, F. B. Nucl. Acids. Res. 28:344-345 (2000)).

Nearly 140 putative inteins have been found from prokaryotes (archaea and eubacteria) and single cell eukaryotes such as algae and yeast, mostly through genome sequencing projects (Perler, F. B. Nucl. Acids. Res. 28:344-345 (2000)). The majority of these inteins mediate maturation of enzymes involved in replication, DNA repair, transcription, or translation. Protein splicing has yet to be observed in a multicellular organism.

Since the discovery of inteins, much has been done to elucidate their functional mechanisms and potential applications. The complete splicing mechanism, consisting of four coupled nucleophilic displacements between three conserved amino acid residues at intein-extein junctions, is reviewed by Noren, C. J. et al. (Angew. Chem. Int. Ed. 39:450-466 (2000)). This protein splicing mechanism has been reconstituted in vivo and in vitro, demonstrating that inteins could be used as powerful tools for protein modification and engineering (Perler, F. B. and Adam, E. Curr. Opin. Biol. 11:377-383 (2000)). Additionally, both trans-splicing and cis-splicing have been studied.

Protein trans-splicing is a reaction that ligates separate proteins into a hybrid molecule, mediated by a pair of split inteins. Therefore, protein trans-splicing offers great advantages over cis-splicing. For example, trans-splicing can permit the synthesis of highly toxic proteins, when a strategy is applied such that single cells only contain a portion of the toxic protein, while the entire toxic protein is synthesized in vitro. Additionally, it may permit expression of a gene from two different loci of a genome or two cellular compartments. To study protein trans-splicing, artificial split inteins have been generated, in which the N-terminal half intein (Int-n) usually contains the critical A and B splicing motifs and the C-terminal half intein (Int-c) contains the C and F motifs. When the half inteins are fused, each half intein being associated with a partial protein, the two partial proteins can be spliced to form a new hybrid product both in vitro and in vivo (Mills, K. V. Proc. Natl. Acad. Sci. USA. 95: 3543-3548 (1998); Southworth, M. W. et al. EMBO. 17:918-926 (1998); Wu, H. et al. Biochimica et Biophysica Acta 187:422-432 (1998); Yamazaki, T. et al. J. Am. Chem. Soc. 120:5591-5592 (1998)). The general utility of these artificial inteins, however, is hindered by a strict requirement for urea treatment to denature and renature the proteins.

The Ssp DnaE inteins are the only known naturally split inteins. This intein class was identified from the split DnaE genes of Synechocystis sp. PCC6803, which encode the catalytic subunit a of DNA polymerase III (Wu, H. et al. Proc. Natl. Acad. Sci. USA. 95:9226-9231 (1998)). The N-terminal half of the DnaE protein containing 774 amino acid residues is fused to the N-terminal 123 amino acid Ssp DnaE intein sequence. The remaining 36 amino acid residues of the C-terminal portion of the Ssp DnaE intein are fused separately to the C-terminal portion of the DnaE protein, containing 423 amino acids. The N-terminal and C-terminal portions are located 745 kb apart on opposite strands of the Ssp PCC6803 genome, although their protein product is an intact catalytic subunit of 1197 amino acid residues lacking any intein sequence due to the intein-mediated protein trans-splicing. In general, efficiency of the protein trans-splicing is usually higher when using Ssp DnaE natural split inteins instead of artificial split inteins (Martin, D. D. et al. Biochemistry. 40:1393-1402 (2001)).

The split Ssp DnaE inteins are also unique in their ability to catalyze the trans-splicing reaction even when two halves of the exteins are foreign proteins. For example, using two compatible plasmids each with an unlinked gene fragment, E. coli was found to be able to: (1) express two gene fragments containing halves of a herbicide-resistant form of the bacterial acetolactate synthase II (ALS II) gene fused to the split intein sequences; and (2) form a herbicide-insensitive enzyme in vivo (Sun, L. et al. Appl. Envir. Micro. 67:1025-1029 (2001)). When a wild type corn ALS gene was similarly used, the expected size of the reconstituted enzyme was formed in vivo (in E. coli) but no evidence was presented as to whether it was functional or whether intein-mediated splicing can occur in plant cells. A similar study was performed, again in E. coli, whereby it was determined that an artificially split 5-enolpyruvylshikimate-3-phosphate synthase (EPSPS) gene (derived from Salmonella typhimurium) could be reassembled as a functional enzyme via intein trans-splicing (Chen et al. Gene 263:3948 (2001).

In both Sun et al., supra, and Chen et al., supra, it is suggested that the split Ssp DnaE inteins are especially applicable for agricultural use of genetically modified plants. More specifically, these authors suggest that trans-splicing technology can be utilized for containment of herbicide resistant transgenes in crops, by expressing inactive gene fragments in separate DNA locations and only allowing protein activity to be generated following trans-splicing. For example, one transgene-intein fragment could be inserted into the nuclear genome, while the other transgene-intein fragment could be fused to an appropriate chloroplast transit peptide and inserted into the chloroplast genome. Thus, two genes could be located in different genomes but their protein products could be spliced in the cytosol. This would prevent the possibility of transferring a functional foreign gene from the transgenic plant to closely related species via cross-pollination, since neither the nucleus nor the chloroplast carries an intact transgene but instead only carries an inactive partial gene. However, these references are silent concerning the methodology that would be necessary for one skilled in the art of plant transgene expression to practice this concept. Further, there has been no demonstration that inteins are able to function in higher organisms, such as plants. There remains a need, therefore, for a method of trans-splicing split inteins in higher plants.

Thus far, the applications for inteins include splicing-dependent protein synthesis, self-cleaving affinity tags for protein purification, use as a novel polypeptide ligation system for protein semisynthesis, segmental labeling of proteins for NMR analysis, addition of fluorescent biosensors, and generation of cyclized proteins (reviewed by Noren, C. J. et al. Angew. Chem. Int. Ed. 39:450-466 (2000)). Despite this wide range of applications, there is yet no reported examples of intein-mediated protein splicing in plants. Although inteins have been identified in yeast nuclear and Chlamydomonus chloroplast genomes, inteins have not yet been found in higher plants or other higher eukaryotes (Perler, F. B. Nucl. Acids. Res. 28:344-345 (2000)). In addition, the art does not teach a method of intein-mediated protein splicing in higher plants.

Plants are increasingly being looked to as platforms for the production of materials foreign to plant systems. Many recombinant proteins have been produced in transgenic plants (Franken et al., Curr. Opin. Biotechnol. 8:411-416 (1997); Whitelam et al., Biotechnol. Genet. Eng. Rev. 11:1-29 (1993)). As the art of genetic engineering advances, it will be possible to engineer plants for the production of a multiplicity of monomers and polymers, currently only available by chemical synthetic means. The accumulation of these materials in various plant tissues will be toxic at some level and it will be useful to tightly regulate the relevant genes to prevent expression in inappropriate plant tissues.

Plant genetic engineering combines modern molecular recombination technology and agricultural crop production. Careful design of transgenic plants will enable production of plants which produce large protein polymers, hybrid protein polymers, and circular protein polymers that are currently impossible for native plant machinery to produce. Further, it will be possible to engineer plants such that they possess certain traits only under selected environmental conditions, in selected plant tissues, at selected development stages, or in selected plant generations.

In the arena of silk-like and fiber-forming proteins, others have demonstrated abundant expression in microbial systems and attempts have also been made to express such proteins in plants. Unfortunately, size limitation is a common problem for both microbial and plant expression. Zhang et al. teach the expression of an elastin-based protein polymer (Gly-Val-Gly-Val-Pro)₁₂₁ (SEQ ID NO:62) in transgenic tobacco plants (Plant Cell Rep. 16(3-4): 174-179 (1996)). Although this represents the expression of a repetitive sequence in plants, the elastin polypeptide bears little resemblance to large silk-like proteins and thus the feasibility of silk-like and fiber-forming protein expression in plants can not be predicted based on this work. Furthermore, methods for the production of complex hybrid protein polymers in plants, where functionality of the hybrid protein can be readily designed and produced in the plant, are not available. One problem to be solved, therefore, is to develop a method of producing large protein polymers, hybrid protein polymers, and circular protein polymers in a variety of plant hosts.

In the arena of regulated transgene expression in plants, few methods provide tight regulation and prevent non-specific expression of transgenes in non-target cells, tissues, or generations. Conditional or regulated expression has been reported in plants based on site-specific recombination systems (i.e., cre-lox, flp-frt, etc.) ([Yadav, PCT Int. Appl. WO 01/36595 A2 (2001); Odell et al., Plant Physiol. 106:447-458 (1994); Odell et al., PCT Int. Appl. WO 9109957 (1991); Surin et al., PCT Int. Appl WO 9737012 (1997); Surin et al., U.S. Pat. No. 2002147168 A1; Ow et al., PCT Int. Appl. WO 9301283 A1 (1992); Russel et al., Mol. Gen. Genet. 234:49-59 (1992); and Hodges et al. (U.S. Pat. No. 6,110,736)]). However, when tested stringently for basal non-specific expression, very few have been strictly specific. Thus, there is a need for an appropriately stringent system suitable for controlling transgenic protein expression and activation of said proteins in commercially-attractive, agricultural crops. This is important where the goal is to produce such high levels of materials in transgenic plants that could otherwise be phytotoxic or adversely affect normal plant development. A second problem to be solved, therefore, is to develop a method suitable for the regulation of transgene expression, such that a particular transgene is expressed only under selected environmental conditions, in selected plant tissues, at selected development stages, or in selected plant generations.

Applicants have solved the stated problems in the present application by applying intein-mediated trans-splicing mechanisms in plants. Applicants have shown that inteins function effectively in plants when they contain plant optimized codons, leading to their self-excision from a protein precursor and ligation of the extein fragments to produce an active protein in the plant. This technique is suitable for a variety of transgene expression applications in plants.

It is often desirable to make use of the smallest DNA and encoded polypeptide segments that are functional, to provide ease of manipulation for the complex constructions and processes that are required for producing genetically altered plants. The IntN portion of the Synechocystis sp. PCC6803 DnaE intein consists of 123 amino acids (Wu, H. et al. (1998) Proc. Natl. Acad. Sci. USA 95, 9226-9231), which is long as compared to the IntC portion of 36 amino acids. It was recently reported that Trichodesmium erythraeum has a DnaE-3 IntN of 102 amino acids; 21 amino acids shorter in its C-terminus than Synechocystis DnaE IntN (Liu, X-Q. & Yang, J. (2003) J. Biol. Chem. 278, 26315-26318). A C-terminal 23 amino acid residue deletionof the Synechocystis DnaE IntN (leaving 100 amino acids) was shown to be functional in E. coli (Ghosh, I. et al. (2001) J. Biol. Chem. 276, 24051-24058). However, this same study found that a 27 amino acid deletion of IntN (leaving 96 amino acids) led to the failure of normal protein splicing in E. coli. Thus there is a need to identify the size of the IntN that is required for protein splicing in plants, so that a smaller than 123 amino acid polypeptide segment can be used, if found to be functional.

Applicants have made a further improvement of intein-mediated trans-splicing in plants, which is the use of a substantially reduced IntN fragment of about 78 amino acids, surprisingly shown by Applicants to be fully functional inplant cells.

### SUMMARY OF THE INVENTION

The present invention provides the application of intein-mediated protein splicing, particularly trans-splicing. The intein-mediated protein splicing of the invention is particularly suitable for use in plants, and the polynucleotides transformed into the plants may be modified with plant optimized codons. The intein-mediated protein splicing of the invention may also be utilized in non-plant eukaryotes, including microbial, yeast, and animal systems.

The invention includes an isolated polynucleotide comprising a nucleotide sequence that encodes a polypeptide comprising an N-terminal portion of the polypeptide (ExtN), a C-terminal portion of the polypeptide (ExtC), and an intein (Int) interposed between the ExtN and the ExtC, wherein at least a portion of the nucleotide sequence has been modified to contain plant optimized codons.

The invention also provides an isolated polynucleotide comprising a nucleotide sequence that encodes a fusion polypeptide consisting of an ExtN, a ExtC, and an Int interposed between the ExtN and the ExtC. In one embodiment, the polynucleotides of the invention encode an intein (Int) that is of bacterial origin. In another embodiment, the polynucleotides further comprise a regulatory sequence, such as a constitutive plant promoter, a plant tissue-specific promoter, or a plant developmental stage-specific promoter. In another embodiment, the polynucleotides encode an intein (Int) that is a naturally split intein consisting of an N-terminal portion (IntN) and a C-terminal portion (IntC). In yet another embodiment, the polynucleotides comprise a nucleotide sequence that comprises (i) an N-nucleotide sequence encoding the ExtN and the IntN and (ii) a C-nucleotide sequence encoding the IntC and the ExtC. In another embodiment, the polynucleotides comprise an N-regulatory sequence that is operably linked to the N-nucleotide sequence and a C-regulatory sequence that is operably linked to the C-nucleotide sequence, and wherein the C-regulatory sequence is interposed between the N-nucleotide sequence and the C-nucleotide sequence. In another embodiment, the ExtN and ExtC together form an active protein. The polynucleotides of the invention also include an isolated polynucleotide comprising a nucleotide sequence that encodes a polypeptide consisting of (i) an ExtN and an IntN or (ii) an ExtC and an IntC, wherein the IntN and the IntC together form a naturally split intein.

In a another embodiment the polynucleotides of the invention comprise a nucleotide sequence encoding an IntN polypeptide that is substantially shorter than known IntN polypeptides. The IntN of this embodiment is about 78 amino acids in length (SEQ ID NO:71 and 72). The IntN includes no amino acids derived from the ExtN that is adjacent to it in the native state.

The invention also includes vectors, host cells, transgenic plants, and seeds that comprise the polynucleotides of the invention.

The invention also includes a method for producing a protein comprising an ExtN and a ExtC. This method comprises the steps of (a) obtaining an N-nucleotide sequence that encodes an N-polypeptide comprising an ExtN and an IntN; (b) obtaining a C-nucleotide sequence that encodes a C-polypeptide comprising an IntC and an ExtC; (c) transforming a plant host with the N-nucleotide sequence and the C-nucleotide sequence such that the plant produces the protein; and (d) optionally recovering the protein. In one embodiment of this method, the step (c) transforming comprises transforming the plant host with a vector that comprises the N-nucleotide sequence and the C-nucleotide sequence. In another embodiment, the step (c) transforming comprises separately transforming the plant host with the N-nucleotide sequence and the C-nucleotide sequence. In yet another embodiment, at least a portion of at least one of the N-nucleotide sequence and the C-nucleotide sequence has been modified to contain plant optimized codons. The IntN and the IntC can together form a naturally split intein and can form an intein of bacterial origin. The protein can consist of the ExtN and the ExtC and, further, can be an active protein.

The invention also includes a method for producing a protein that comprises an ExtN and a ExtC. This method comprises the steps of (a) transforming an N-plant host with an N-polynucleotide comprising an N-nucleotide sequence that encodes an N-polypeptide comprising the ExtN and an IntN, such that the N-plant host produces the N-polypeptide; (b) transforming a C-plant host with a C-polynucleotide comprising a C-nucleotide sequence that encodes a C-polypeptide comprising a IntC and the ExtC, such that the C-plant host produces the C-polypeptide; and (c) crossing the N-plant host and the C-plant host to obtain a progeny of the N-plant host and the C-plant host, wherein the progeny comprises the protein. In one embodiment of this method, at least a portion of at least one of the N-nucleotide sequence and the C-nucleotide sequence has been modified to contain plant optimized codons. In another embodiment, the IntN and the IntC form a naturally split intein. In yet another embodiment, the (a) transforming comprises introducing an N-vector into the N-plant host and wherein the N-vector comprises the N-nucleotide sequence, and wherein the (b) transforming comprises introducing a C-vector into the C-plant host and wherein the C-vector comprises the C-nucleotide sequence.

The invention further includes a method for producing a protein comprising an ExtN and a ExtC. This method comprises the steps of (a) transforming an N-plant host with an N-polynucleotide comprising an N-nucleotide sequence that encodes an N-polypeptide comprising the ExtN and an IntN, such that the N-plant host produces the N-polypeptide; (b) transforming a C-plant host with a C-polynucleotide comprising a C-nucleotide sequence that encodes a C-polypeptide comprising a IntC and the ExtC, such that the C-plant host produces the C-polypeptide; (c) isolating the N-polypeptide from the N-plant host and the C-polypeptide from the C-plant host; and (d) combining the N-polypeptide and the C-polypeptide in vitro to obtain the protein. In one embodiment of this method, at least a portion of at least one of the N-nucleotide sequence and the C-nucleotide sequence has been modified to contain plant optimized codons. In another embodiment, the step (a) transforming comprises introducing an N-vector into the N-plant host and wherein the N-vector comprises the N-nucleotide sequence, and wherein the (b) transforming comprises introducing a C-vector into the C-plant host, the C-vector comprising the C-nucleotide sequence.

In the methods of the invention, the plant host can be a plant, a plant derived tissue, or a plant cell. The plant host can also be selected from food plants, non-food plants, arboreous plants, and aquatic plants.

The invention further provides a transgenic plant that produces an active protein comprising an ExtN and a ExtC, wherein the protein is produced from a polynucleotide comprising a nucleotide sequence that encodes the ExtN, the ExtC, and an intein interposed between the ExtN and the ExtC. The invention also provides a transgenic plant that expresses a polypeptide consisting of (i) an ExtN and an IntN or (ii) an ExtC and an IntC, wherein the IntN and the IntC together form an intein, and wherein the ExtN and the ExtC together form an active protein. In one embodiment of the transgenic plant of the invention, at least a portion of the nucleotide sequence has been modified to contain plant optimized codons. In another embodiment, the protein is expressed in at least one of a leaf, a root, a stem, a flower, a fruit, or a seed of the plant.

### BRIEF DESCRIPTION OF FIGURES AND SEQUENCE DESCRIPTIONS

FIG. 1A is a plasmid map of pHGUSH, in which the GUS fragment contains a 6×His peptide at the N-terminus and a 6×His peptide with a stop codon integrated at the C-terminus. FIG. 1B shows the amino acid sequence derived from the HGUSH coding region.

FIG. 2A is a plasmid map of pGUSN-Intn, which contains a GUSn/Intn fusion whose sequence is shown in FIG. 2B. FIG. 2C is a plasmid map of pGUSN-Intn(6), which contains a GUSn/Intn(6) fusion whose sequence is shown in FIG. 2D.

FIG. 3A is a plasmid map of pIntC-GUSc, which contains a Intc/GUSc fusion whose sequence is shown in FIG. 3B.

FIG. 4A is a plasmid map of pGYV1/GUS, upon which expression plasmids were designed. This vector contains expression cassettes of 35S-Pro::GUS::NOS-Ter and NOS-Pro::NPTII::OCS-Ter. FIG. 4B is a plasmid map of pGYV1/GUSM, derived from pGYV1/GUS.

FIG. 5A is a plasmid map of p35SGIN, containing an expression cassette of NOS-Pro::NPTII::OCS-Ter for transgenic plant selection and expression Cassette I (35S-Pro::GUSn/Intn::NOS-Ter) for GUSn/Intn fusion protein expression. FIG. 5B is a plasmid map of p35SGIN(6), containing an expression cassette of NOS-Pro::NPTII::OCS-Ter for transgenic plant selection and expression Cassette II (35S-Pro::GUSn/Intn(6)::NOS-Ter) for GUSn/Intn(6) fusion protein expression. FIG. 5C is a plasmid map of p35SIGC(−)-Bar, a binary vector containing an expression cassette of NOS-Pro::Bar::NOS-Ter for transgenic plant selection and expression Cassette III (35S-Pro::Intc/GUSc::NOS-Ter) for Intc/GUSc fusion protein expression.

FIG. 6A is a plasmid map of p35SGIN-35SIGC, containing an expression cassette of NOS-Pro::NPTII::OCS-Ter for transgenic plant selection. It also has expression Cassette I (35S-Pro::GUSn/Intn::NOS-Ter) for GUSn/Intn fusion protein expression and expression Cassette III (35S-Pro::Intc/GUSc::NOS-Ter) for Intc/GUSc fusion protein expression.

FIG. 6B is a plasmid map of p35SGIN(6)-35SIGC, containing an expression cassette of NOS-Pro::NPTII::OCS-Ter for transgenic plant selection. It also has expression Cassette II (35S-Pro::GUSn/Intn(6)::NOS-Ter) for GUSn/Intn(6) fusion protein expression and expression Cassette III (35S-Pro::Intc/GUSc::NOS-Ter) for Intc/GUSc fusion protein expression.

FIGS. 7, 8A, 12, and 15 show GUS staining results on transgenic Arabidopsis plants, in various stages of development. FIG. 8B depicts staining of seeds from wildtype and transformed plants.

FIGS. 9A, B, and C and 13A and B show PCR results from genomic DNA for transgene integration into transgenic Arabidopsis plants.

FIGS. 10, 14A, and 16A show RNA filter hybridization assay results.

FIGS. 11A and B, 14B, and 16B show protein filter immunoblot assays detected with various antibodies.

FIG. 17 shows GUS staining results on 2-week old leaves of transgenic tobacco, soy, pea, maize, and barley plants.

FIGS. 18A and 18B show transient co-expression of split Cre recombinase elements results in site specific recombination and activation of the GUS reporter gene. FIG. 18C illustrates the molecular events that must occur for intein-mediated protein splicing of the Cre recombinase, thereby permitting excision of the blocking fragment and expression of the GUS reporter.

FIG. 19A shows the expression cassettes used to analyze the length of IntN required for protein splicing in plant cells and 19B details each deletion point in the amino acid sequence and shows the presence of motif A and motif B.

The following sequence descriptions and sequences listings attached hereto comply with the rules governing nucleotide and/or amino acid sequence disclosures in patent applications as set forth in 37 C.F.R. §1.821-1.825. The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

SEQ ID NOs:1 and 2 are the native amino acid sequence of the split intein DnaE from Synechocystis sp. PCC6803.

SEQ ID NOs:3-21 represent overlapping oligomers, containing plant optimized codons, used for synthesis of the split intein DnaE from Synechocystis sp. PCC6803.

SEQ ID NO:22 is the nucleotide sequence for the split intein Ssp DnaE Int-n, containing plant optimized codons, and named as PInt-n.

SEQ ID NO:23 is the amino acid sequence encoded by the PInt-n sequence of SEQ ID NO:22.

SEQ ID NO:24 is the nucleotide sequence for the split intein Ssp DnaE Int-c, containing plant optimized codons, and named as PInt-c.

SEQ ID NO:25 is the amino acid sequence encoded by the PInt-c sequence of SEQ ID NO:24.

SEQ ID NOs:26 and 27 are PCR primers HGUSH-n and GUSC-Bam, used for modification of the GUS gene.

SEQ ID NO:28 is the amino acid sequence encoding the GUS protein with 6×His tags at both N- and C-termini (the HGUSH coding region).

SEQ ID NOs:29 and 30 are PCR primers GUS-N2 and GUS-C2, used to confirm the sequence of the HGUSH region in PHGUH.

SEQ ID NOs:31 and 32 are PCR primers trpN-SstII and trpC-SstII.

SEQ ID Nos:33-37 are PCR primers designed for PCR-directed recombination to create in-frame fusions of GUS-n/Int-n and Int-c/GUS-c.

SEQ ID NO:38-40 are the amino acid sequences for the GUSn/Intn, GUS/Intn(6), and Intc/GUSc fusion proteins, respectively.

SEQ ID NOs:41 and 42 are PCR primers KNNOS and NOSXS.

SEQ ID NOs:43, 49, 50, 56, 58, 60, and 61 are various linker sequences used in vector design.

SEQ ID NOs:44-47 are the primers used as PH820, PH821, PH824, and PH825, respectively.

SEQ ID NO:48 is a 3034 bp Asp 718 fragment containing a 35S-CreN-IntN ocs gene in plasmid pGV947.

SEQ ID NOs:51-54 are the primers used as PH826, PH827, PH822, and PH823, respectively.

SEQ ID NO:55 is the 2873 bp Asp 718 bp fragment containing 35S:IntC-CreC:3′ocs in plasmid pGV951.

SEQ ID NO:57 is the 5449 bp Sal I-Hind III fragment containing the blocked GUS reporter gene for Cre-Lox excision in plasmid pGV801.

SEQ ID NO:59 is the Lox P sequence.

SEQ ID NO:62 is the elastin-based protein polymer synthesized by Zhang et al. (Plant Cell Rep. 16(3-4):174-179 (1996)).

SEQ ID NO:63 is the coding sequence introduced by oligomer HGUSH-n.

SEQ ID NO:64 is the insertion sequence in pGY101 (a pBluscript-based plasmid).

SEQ ID NO:65 are the residues deleted from IntN to create the GUSn/Intn(6) fusion.

SEQ ID NO:66 is the 12-amino acid N-terminal extension added to the GUS ORF.

SEQ ID NOs:67-70 are primers Nint-1, Nint-2, Nint-3, and Nint-4 used for deletion mutagenesis of the Intn coding sequence.

SEQ ID NO:71 is the 78 amino acid IntN sequence.

SEQ ID NO:72 is the nucleotide sequence encoding the 78 amino acid IntN.

SEQ ID NO:73 is the amino acid sequence of Motif A encompassed in the Synechocystis IntN sequence.

SEQ ID NO:74 is the amino acid sequence of Motif B encompassed in the Synechocystis IntN sequence.

### DETAILED DESCRIPTION OF THE INVENTION

The present invention provides constructs and methods to introduce a protein splicing mechanism into plants by employing inteins and transgenes. Inteins function effectively in plants when they contain plant optimized codons, leading to their self-excision from a protein precursor and ligation of the extein fragments to produce a mature or active protein in the plant. This mechanism can be utilized to assemble exteins into large protein polymers (including structural proteins and bioactive proteins), hybrid protein polymers, and circular protein polymers. Further, by selectively choosing promoters responsive to various inducers, plant tissues, or plant developmental states, it is possible to control the protein splicing mechanism so as to produce complex mature and active protein products under selected environmental conditions, in selected plant tissues, at selected development stages, or in selected plant generations. This permits use of the intein-mediated protein splicing reaction as a means to activate regulatory protein factors and enzymes, and thus control gene expression and metabolism. The present invention and its embodiments therefore can benefit plant-based protein polymer production methods and have use in agronomic practice for various other agricultural and industrial applications.

Definitions

The following terms and definitions shall be used to fully understand the specification and claims.

“Polymerase chain reaction” is abbreviated PCR.

“Open reading frame” is abbreviated ORF.

The term “intein-mediated protein splicing” refers to the process whereby an intein catalyzes its removal from a protein precursor, permitting synthesis of a mature, active protein. When a pair of split inteins are involved in the splicing process, the mature and active protein is formed from two separate protein precursors. This splicing process is defined as “trans-protein splicing”.

“Intein” refers to an in-frame intervening sequence in a protein precursor. The intein disrupts the coding region of a gene, until it catalyzes its own excision from the protein precursor through a post-translational protein splicing process to yield the free intein and a mature protein. This definition encompasses mini-inteins, synthetic inteins, split inteins, and optimized codon-modified inteins.

A “split intein” is comprised of two distinct polypeptides or proteins, referred to as the “N-terminal” or N-intein (abbreviated as IntN, Intn or Int-n) and the “C-terminal” or C-intein (abbreviated as IntC, Intc or Int-c) because of their homology to the N-terminal and C-terminal regions of non-split inteins, respectively. Together IntN and IntC polypeptides, when operably linked to foreign polypeptides, possess all necessary functionality to complete a trans-protein splicing reaction, whereby the two foreign “extein” fragments are ligated together by formation of a peptide bond. DNA sequences encoding IntN and IntC may be separated by many kilobases of nucleotides in a genome or on different chromosomes.

The intein (or IntN in the case of a split intein) is flanked immediately upstream by a N-terminal portion of a protein precursor known as the N-extein (abbreviated as N-extein or extN). In like manner, the intein (or IntC in the case of a split intein) is flanked immediately downstream by the C-terminal portion of a protein precursor known as the C-terminal extein (abbreviated as C-extein or extC). The N-extein and C-extein are ligated together by a peptide bond to form a mature, active protein in the protein splicing process.

An “intein cassette” refers to a synthetic construct that minimally includes an intein or a portion thereof, and an extein. This encompasses constructs which have the structure: ExtN-Int-ExtC, wherein: ExtN is the N-terminal portion of the polypeptide precursor; Int is an intein; and ExtC is the C-terminal portion of the polypeptide precursor. Additionally, an intein cassette also encompasses constructs that have the structures of ExtN-IntN and IntC-ExtC. In this case, the intein is a split intein, composed of an N-terminal portion of a split intein (IntN) or a C-terminal portion of a split intein (IntC)). In all cases, an intein cassette may possess intervening sequences between the intein sequence and extein fragment that are destined to produce a mature, active protein. These intervening sequences may include, for example, regulatory sequences (e.g., promoters and 3′ terminators) or blocking sequences.

An “N-nucleotide sequence” hereinafter refers to a split intein cassette that encodes the N-terminal portion of the polypeptide precursor (or “N-polypeptide”), and that minimally includes ExtN and IntN. In a preferred embodiment, ExtN and IntN are a fusion polypeptide in which the ExtN protein is fused at its C-terminus to the N-terminus of IntN protein.

A “C-nucleotide sequence” will hereinafter refer to a nucleotide sequence that encodes the C-terminal portion of a protein precursor (or “C-polypeptide”), and that minimally includes IntC and ExtC. In a preferred embodiment, IntC and ExtC are a fusion polypeptide in which the IntC protein is fused at its C-terminus to the N-terminus of ExtC protein.

A “N-vector” refers to a vector that contains a N-nucleotide sequence. A “C-vector” refers to a vector that contains a C-nucleotide sequence.

A “N-polypeptide” refers to a protein precursor that is produced from an N-nucleotide sequence, while a “C-polypeptide” refers to a protein precursor that is produced from a C-nucleotide sequence.

A “N-plant host” refers to a plant that has been transformed with an N-nucleotide sequence. In like manner, a “C-plant host” refers to a plant that has been transformed with a C-nucleotide sequence.

The “fusion protein” or “fusion polypeptide” of the invention refers to two or more proteins or polypeptides that are fused together. Examples of fusion polypeptides include polypeptides having the contiguous sequence of Ext-Int-Ext, ExtN-IntN-IntC-ExtC, ExtN-IntN, IntC-ExtC, or ExtN-ExtC.

“Gene” refers to a nucleic acid fragment that expresses mRNA, functional RNA, or specific protein, including regulatory sequences. The term “native gene” refers to gene as found in nature. The term “chimeric gene” refers to any gene that contains: 1) DNA sequences, including regulatory and coding sequences, that are not found together in nature; or 2) sequences encoding parts of proteins not naturally adjoined; or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism.

“Synthetic genes” can be assembled from oligonucleotide building blocks that are chemically synthesized using procedures known to those skilled in the art. These building blocks are annealed and ligated to form gene segments that are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available. “Plant optimized codons”, therefore, refers to the selection and use of optimized codons in plants. This bias can be targeted for either monocot or dicot plants, as necessary.

“Coding sequence” refers to a DNA or RNA sequence that codes for a specific amino acid sequence and excludes the non-coding sequences. The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters; however, some suitable regulatory sequences useful in the present invention will include, but are not limited to: constitutive plant promoters, plant tissue-specific promoters, plant developmental stage-specific promoters, inducible plant promoters and viral promoters.

The “3′ region” or “3′ terminator” means the 3′ non-coding regulatory sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, RNA processing or stability, or translation of the associated coding sequence (e.g. for a recombinase, a transgene, etc.).

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter”also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue-specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

“Constitutive promoter” refers to promoters that direct gene expression in all tissues and at all times. “Regulated promoter” refers to promoters that direct gene expression not constitutively but in a temporally- and/or spatially-regulated manner and include tissue-specific, developmental stage-specific, and inducible promoters. The constitutive and regulated promoters include natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered; numerous examples may be found in the compilation by Okamuro et al. (Biochemistry of Plants 15:1-82 (1989)). Since in most cases the exact boundaries of regulatory sequences have not been completely defined, DNA fragments of different lengths may have identical promoter activity. Typical regulated promoters useful in plants include but are not limited to safener-inducible promoters, promoters derived from the tetracycline-inducible system, promoters derived from salicylate-inducible systems, promoters derived from alcohol-inducible systems, promoters derived from the glucocorticoid-inducible system, promoters derived from pathogen-inducible systems, and promoters derived from ecdysome-inducible systems.

“Tissue-specific promoter” refers to regulated promoters that are not expressed in all plant cells but only in one or more cell types in specific organs (such as leaves, shoot apical meristem, flower, or seeds), specific tissues (such as embryo or cotyledon), or specific cell types (such as leaf parenchyma, pollen, egg cell, microspore- or megaspore mother cells, or seed storage cells). These also include “developmental stage-specific promoters” that are temporally regulated, such as in early or late embryogenesis, during fruit ripening in developing seeds or fruit, in fully differentiated leaf, or at the onset of senescence. It is understood that the developmental specificity of the activation of a promoter and, hence, of the expression of the coding sequence under its control, in a transgene may be altered with respect to its endogenous expression. For example, when a transgene under the control of a floral promoter is transformed into a plant, even when it is the same species from which the promoter was isolated, the expression specificity of the transgene will vary in different transgenic lines due to its insertion in different locations of the chromosomes.

“Plant developmental stage-specific promoter” refers to a promoter that is expressed not constitutively but at specific plant developmental stage or stages. Plant development goes through different stages and in context of this invention the germlne goes through different developmental stages starting, say, from fertilization through development of embryo, vegetative shoot apical meristem, floral shoot apical meristem, anther and pistil primordia, anther and pistil, micro- and macrospore mother cells, and macrospore (egg) and microspore (pollen).

“Inducible promoter” refers to those regulated promoters that can be turned on in one or more cell types by a stimulus external to the plant, such as a chemical, light, hormone, stress, or a pathogen.

“Promoter activation” means that the promoter has become activated (or turned “on”) so that it functions to drive the expression of a downstream genetic element. Constitutive promoters are continually activated. A regulated promoter may be activated by virtue of its responsiveness to various external stimuli (inducible promoter), or developmental signals during plant growth and differentiation, such as tissue specificity (floral specific, anther specific, pollen specific seed specific etc) and development-stage specificity (vegetative or floral shoot apical meristem-specific, male germlne specific, female germlne specific etc).

“Conditionally activating” refers to activating a transgenic protein that is normally not expressed. In context of this invention it refers to intein-mediated protein splicing either by a cross or, if it is inducible, also by an inducer, to produce a mature active protein.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences can be operably-linked to regulatory sequences in sense or antisense orientation. “Unlinked” means that the associated genetic elements are not closely associated with one another and function of one does not affect the other.

“Genetically linked” refers to physical linkage of transgenic cassettes such that they co-segregate in progeny. “Genetically unlinked” refers to the lack of physical linkage of transgenic cassettes such that they do not co-segregate in progeny.

“Expression” refers to the transcription and stable accumulation of sense (mRNA) or functional RNA. Expression may also refer to the production of active protein. “Overexpression” refers to the level of expression in transgenic organisms that exceeds levels of expression in normal or untransformed organisms. “Altered levels” refers to the level of expression in transgenic organisms that differs from that of normal or untransformed organisms. “Conditional and transient expression” refers to expression of an active transgenic protein only in the selected generation or two. In context of this invention, expression of a mature or active transgenic protein is triggered by intein-mediated protein splicing, which may only occur when the complete intein (or IntN and IntC) is co-localized within the same compartment in plant cells.

“Constitutive expression” refers to expression using a constitutive or regulated promoter. “Conditional” and “regulated expression” refer to expression controlled by a regulated promoter. “Transient” expression in the context of this invention refers to expression only in specific developmental stages or tissue in one or two generations. Finally, “non-specific expression” refers to constitutive expression or low level, basal (‘leaky’) expression in nondesired cells, tissues, or generation.

“Mature” protein or “active” protein refers to a polypeptide that has undergone post-translational processing and intein-mediated protein splicing processing, when possible. The mature or active protein no longer has any pre- or propeptides or inteins present, as these are removed from the primary translation product. It should be understood that a protein precursor which contains an intein fragment is fully transcribed into mRNA and translated into protein. However, the protein so produced is an inactive transgenic protein, due to the presence of the intein fragment. Only upon removal of the “blocking” intein fragment via intein-mediated protein splicing may an active transgenic protein be produced.

A “hybrid protein” refers to a protein with multiple functions, created by the artificial combination between a functional peptide and another functional molecule (e.g., another functional peptide) using the protein splicing mechanism. Typically, this hybrid protein is composed of amino acid sequences derived from more than one gene, yet the coding DNA sequences are “in frame” within a gene, thereby permitting complete expression of both “original” functional peptides.

The term “altered plant trait” means any phenotypic or genotypic change in a transgenic plant relative to the wildtype or non-transgenic plant host.

“Production tissue” refers to mature, harvestable tissue consisting of non-dividing, terminally-differentiated cells. It excludes young, growing tissue consisting of germline, meristematic, and not-fully-differentiated cells.

“Germline” refers to cells that are destined to be gametes. Thus, the genetic material of germlne cells is heritable. “Common germline” refers to all germlne cells prior to their differentiation into the male and female germlne cells and, thus, includes the germlne cells of developing embryo, vegetative SAM, floral SAM, and flower. “Male germline” refers to cells of the sporophyte (anther primordia, anther, microspore mother cells) or gametophyte (microspore, pollen) that are destined to be male gametes (sperm) and the male gametes themselves. “Female germline” refers to cells of the sporophyte (pistil primordial pistil, ovule, macrospore mother cells) or gametophyte (macrospore, egg cell) that are destined to be female gametes or the female gametes themselves.

“Transformation” refers to the transfer of a foreign gene into the genome of a host organism. Examples of methods of plant transformation include Agrobacterium-mediated transformation (De Blaere et al. Meth. Enzymol. 143:277 (1987)) and particle-accelerated or “gene gun” transformation technology (Klein et al. Nature (London) 327:70-73 (1987); U.S. Pat. No. 4,945,050). The terms “transformed”, “transformant” and “transgenic” refer to plants or calli that have been through the transformation process and contain a foreign gene integrated into their chromosome. The term “untransformed” refers to normal plants that have not been through the transformation process.

“Stably transformed” refers to cells that have been selected and regenerated on a selection media following transformation.

“Genetically stable” and “heritable” refer to chromosomally-integrated genetic elements that are stably maintained in the plant and stably inherited by progeny through successive generations.

“Wild-type” refers to the normal gene, virus, or organism found in nature without any known mutation.

“Genome” refers to the complete genetic material of an organism.

“Genetic trait” means a genetically determined characteristic or condition, which is transmitted from one generation to another. “Homozygous” state means a genetic condition existing when identical alleles reside at corresponding loci on homologous chromosomes. In contrast, “heterozygous” state means a genetic condition existing when different alleles reside at corresponding loci on homologous chromosomes. A “hybrid” refers to any offspring of a cross between two genetically unlike individuals. “Inbred” or “inbred lines” or “inbred plants” means a substantially homozygous individual or variety. This results by the continued mating of closely related individuals, especially to preserve desirable traits in a stock.

“Selfing” or “self fertilization” refers to the transfer of pollen from an anther of one plant to the stigma (a flower) of that same said plant. Selfing of a hybrid (F1) results in a second generation of plants (F2).

“Primary transformant” refer to transgenic plants that are of the same genetic generation as the tissue which was initially transformed (i.e., not having gone through meiosis and fertilization since transformation). Thus, primary transformants usually refer to the “T0 generation”. But, in flower transformation, “primary transformant” refers to the T1 generation instead, because the transformants can only be identified from the T1 generation of plants.

“Secondary transformants” and the “T₁, T₂, T₃, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

The terms “plasmid” and “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Typically, a “vector” is a modified plasmid that contains additional multiple insertion sites for cloning and an “expression cassette” that contains a DNA sequence for a selected gene product (i.e., a transgene) for expression in the host cell. This “expression cassette” typically includes a 5′ promoter region, the transgene ORF, and a 3′ terminator region, with all necessary regulatory sequences required for transcription and translation of the ORF. Thus, integration of the expression cassette into the host permits expression of the transgene ORF in the cassette.

As used herein the following abbreviations will be used to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamine acid | Glu | E |
| Glutamine or glutamic acid | Glx | Z |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Standard recombinant DNA and molecular cloning techniques used here are well known in the art and are described by Sambrook, J., Fritsch, E. F. and Maniatis, T., Molecular Cloninq: A Laboratory Manual, 2nd ed., Cold Spring Harbor Laboratory: Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience (1987).

The present invention provides constructs and methods to introduce an intein-mediated protein splicing mechanism into plants by employing transgenes and inteins with plant optimized codons. This mechanism is useful to assemble exteins into large hybrid and circular protein polymers, and/or to control expression of the transgene. By selectively choosing promoters (responsive to various inducers or functional in various plant tissues or during various plant developmental states), it is possible to control the protein splicing mechanism so as to produce complex mature and active protein products under selected environmental conditions, in selected plant tissues, at selected development stages, or in selected plant generations.

The Intein Cassette

The invention makes use of a variety of specialized expression constructs referred to herein as intein cassettes. Each intein cassette comprises an intein and an extein, wherein at least a portion thereof contains plant optimized codons. Intein cassettes have a variety of various structures, including ExtN-Int-ExtC, ExtN-IntN, and IntC-ExtC. Additionally the intein cassette may comprise a number of other components, such as specific regulatory signals.

Promoters

The present invention can make use of a variety of plant promoters to drive the expression of the intein cassettes of the invention. Regulated expression of each intein cassette is possible by placing the intein cassette under the control of promoters that may be conditionally regulated. Any promoter functional in a plant will be suitable including, but not limited to: constitutive plant promoters, plant tissue-specific promoters, plant development-specific promoters, inducible plant promoters, and flower-specific promoters. Additionally, viral promoters, male germline-specific promoters, female germline-specific promoters, and vegetative shoot apical meristem-specific promoters should be useful in the present invention.

Commonly used constitutive promoters in plants include the Arabidopsis SAMS (Mordhorst, A.P. et al. Genetics. 149(2):549-63 (1998)), Arabidopsis UBQ (ubiquitin) (Sun, C. K., and Callis, J. Plant May; 11(5):1017-27 (1997)), CaMV 35S, Ti Plasmid OCS (octopine synthase), and Ti plasmid NOS (nopaline synthase).

Several tissue-specific and/or development-specific regulated genes and/or promoters have been reported in plants. These include genes encoding the seed storage proteins (e.g., napin, cruciferin, beta-conglycinin [cotyledon specific from soy], and phaseolin [cotyledon-specific from common bean]), zein or oil body proteins (e.g., the endosperm-specific maize zein and the embryo-specific brassica oleosin), or genes involved in fatty acid biosynthesis (e.g., acyl carrier protein, stearoyl-ACP desaturase, and fatty acid desaturases (fad 2-1)), and other genes expressed during embryo development (e.g., Bce4, see, for example, EP 255378 and Kridl et al., Seed Science Research 1:209-219 (1991)). Particularly useful for seed-specific expression is the pea vicilin promoter (Czako et al., Mol. Gen. Genet. 235(1): 33-40 (1992)).

Other useful promoters for expression in mature leaves are those that are switched on at the onset of senescence, such as the SAG promoter from Arabidopsis (Gan et al., Science 270(5244):1986-8 (1995)). Root or tuber specific promoters are also known, such as tobacco TobRB7, wheat lamda pox1 (peroxidase), and potato patatin B33. Flower or “floral” -specific promoters are those whose expression occurs in the flower or flower primordia (e.g., petunia chsA (chalcone synthase)). Anther-specific promoters (e.g., Arabidopsis A9 for tapetum-specific) and pollen-specific promoters (maize Pex1 [pollen extensin-like protein] and tomato Lat52 [Twell et al. Trends in Plant Sciences 3:305 (1998)] for pollen-specific) have also been identified and will be useful in the present invention. Recently, cDNA clones representing genes apparently involved in tomato pollen (McCormick et al., Tomato Biotechnology (1987) Alan R. Liss: NY) and pistil (Gasser et al., Plant Cell 1:15-24 (1989)) interactions have also been isolated and characterized.

A class of fruit-specific promoters expressed at or during anthesis through fruit development, at least until the beginning of ripening, is discussed in U.S. Pat. No. 4,943,674, the disclosure of which is hereby incorporated by reference. cDNA clones that are preferentially expressed in cotton fiber have been isolated (John et al., Proc. Natl. Acad. Sci. U.S.A. 89(13): 5769-73 (1992)). cDNA clones from tomato displaying differential expression during fruit development have been isolated and characterized (Mansson et al., Mol. Gen. Genet. 200:356-361 (1985); Slater et al., Plant Mol. Biol. 5:137-147 (1985)). The promoter for polygalacturonase gene is active in fruit ripening. The polygalacturonase gene is described in U.S. Pat. No. 4,535,060 (issued Aug. 13, 1985), U.S. Pat. No. 4,769,061 (issued Sep. 6, 1988), U.S. Pat. No. 4,801,590 (issued Jan. 31, 1989) and U.S. Pat. No. 5,107,065 (issued Apr. 21, 1992), which disclosures are incorporated herein by reference.

Mature plastid mRNA for psbA (one of the components of photosystem II) reaches its highest level late in fruit development, in contrast to plastid mRNAS for other components of photosystem I and II which decline to nondetectable levels in chromoplasts after the onset of ripening (Piechulla et al., Plant Mol. Biol. 7:367-376 (1986)). A second promoter identified to function efficiently in chloroplasts is the tobacco Prrn promoter, a plastid rRNA operon promoter. In like manner, mitochondria promoters are also known, such as the wheat cox2 (cytochrome oxidase subunit 2) and soy atp9 (ATP snythase subunit 9) promoters. Other examples of tissue-specific promoters include those that direct expression in leaf cells following damage to the leaf (e.g., from chewing insects), in tubers (e.g., patatin gene promoter), and in fiber cells (e.g., the E6 developmentally-regulated fiber cell protein (John et al., Proc. Natl. Acad. Sci. U.S.A. 89(13): 5769-73 (1992),). The E6 gene is most active in fiber, although low levels of transcripts are found in leaf, ovule and flower.

The tissue-specificity of some “tissue-specific” promoters may not be absolute and may be tested by one skilled in the art using the diphtheria toxin sequence. One can also achieve tissue-specific expression with “leaky” expression by a combination of different tissue-specific promoters (Beals et al., Plant Cell, 9: 1527-1545 (1997)). Other tissue-specific promoters can be isolated by one skilled in the art (see U.S. Pat. No. 5,589,379).

Similarly, several inducible promoters (“gene switches”) have been reported. Many are described in the review by Gatz (Current Opinion in Biotechnology, 7: 168-172 (1996); Gatz, C. Annu. Rev. Plant Physiol. Plant Mol. Biol. 48: 89-108 (1997)). These include the tetracycline repressor system, Lac repressor system, copper-inducible systems (e.g., yeast acel), salicylate-inducible systems (such as the PR1a system), glucocorticoid—(Aoyama T. et al., N-H Plant Journal 11:605-612 (1997)), estradioal—(e.g., “XVE”), and ecdysome-inducible systems. Also, included are the benzene sulphonamide—(U.S. Pat. No. 5,364,780) and alcohol—(WO 97/06269 and WO 97/06268) inducible systems and glutathione S-transferase promoters. Other studies have focused on genes inducibly regulated in response to environmental stress or stimuli such as increased salinity, drought, pathogen, and wounding (Graham et al., J. Biol Chem. 260:6555-6560 (1985); Graham et al., J. Biol. Chem. 260:6561-6554 (1985); Smith et al., Planta 168:94-100 (1986)). Specific promoters include the wound/pathogen inducible Asparagua officinalis AoPR1 and tomato PI-1 (proteinase inhibitor-1) promoters and the water-stress inducible tobacco osmotin promoter and rice rab-16A promoter. Accumulation of a metallocarboxypeptidase-inhibitor protein has been reported in leaves of wounded potato plants (Graham et al., Biochem Biophys Res Comm 101:1164-1170 (1981)). Other plant genes that have been reported to be induced are methyl jasmonate, elicitors, heat-shock (e.g., Arabidopsis HSP18.2, soy Gmbsp 17-E), anerobic stress, or herbicide safeners (e.g., maize In2-2).

Inteins

The present invention provides intein-mediated protein splicing for use in assembly of protein polymers and the regulated expression of transgenic proteins. Protein precursors which contain an intein fragment are fully transcribed into mRNA and translated into protein. However, the protein so produced is an incomplete or inactive transgenic protein, due to the presence of the intein fragment. Only upon removal of the “blocking” intein fragment via intein-mediated protein splicing may a mature or active transgenic protein be produced. This intein-mediated splicing mechanism, consisting of four coupled nucleophilic displacements between three conserved amino acid residues at intein-extein junctions (reviewed by Noren, C. J. et al. Angew. Chem. Int. Ed. 39:450-466 (2000)), allows the self-excision of the blocking intein fragment from the protein precursor, thereby permitting production of a mature or active protein. Therefore, the conditional excision of the blocking fragment by intein-mediated protein splicing controls the production of the mature or active transgenic protein.

Although only 140 putative inteins have been found thus far in prokaryotes (archaea and eubacteria) and single cell eukaryotes such as algae and yeast (Perler, F. B. Nucl. Acids. Res. 28:344-345 (2000)), it is expected that many more will be identified in future genome sequencing projects. The present invention is not limited by the choice of intein. Instead the invention embodies all those inteins which are capable of catalyzing said self-excision from a protein precursor to yield an active protein. This class of inteins thus embodies naturally discovered inteins from prokaryotes and eukaryotes (including multicellular organisms, if discovered), and synthetic inteins. These synthetic inteins can be modified to contain optimized codons for a specific host organism, as in the present invention, can be modified to function as split inteins, or can be modified to function as mini-inteins (whereby the central homing region of the intein is deleted).

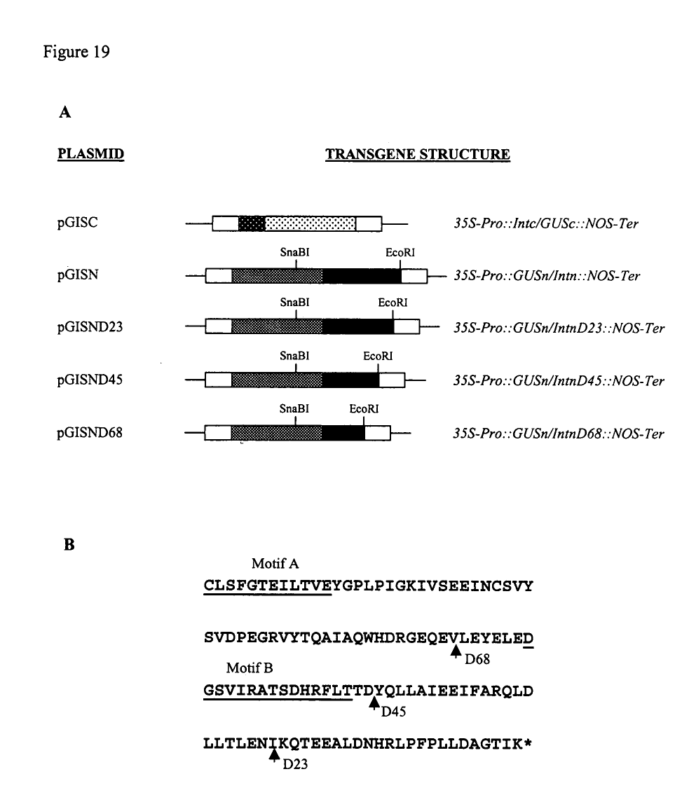

In addition, the intein sequence can be shortened if it is found that the entire sequence (after removal of the central homing region) is not necessary to mediate the trans-splicing of ExtN and ExtC. For example, Applicants made deletions of the polynucleotide encoding the 123 amino acid long IntN (N-terminal portion of the intein) and found that a 78 amino acid long IntN was sufficient for splicing to occur at a level comparable to the full sequence. The 45 amino acids were removed from the C-terminal end of IntN. However, removal of 68 amino acids from the C-terminus, leaving an IntN of 55 amino acids, resulted in extremely low to no trans-splicing activity. Thus the functional portion IntN is shorter than the entire 123 amino acids, longer than 55 amino acids, with an approximately 78 amino acid length being preferred. The 78 amino acid IntN DNA segment includes two motifs (motif A and motif B, FIG. 19B) that are conserved in known inteins. In the 55 amino acid non-functional intein one of these motifs was deleted. Thus a DNA segment sufficiently long to include the two conserved motifs and natural intervening sequence is adequate for function in plant cells. The two conserved motifs, called motif A and motif B, have high identities between the IntN sequences of the two known naturally split inteins (Liu, X-Q and Yang, J 2003 J of Biol Chem 278:26315-18). The two motif A's differ in only 4 out of 12 amino acids (SEQ ID NOs: 73 and 75). The two motif B's differ in 5 out of 15 amino acids (SEQ ID NOs: 74 and 76). It should be understood that the two motif A sequences of SEQ ID NOs:73 and 75 are equivalent compositions of motif A, while the two motif B sequences of SEQ ID NOs:74 and 76 are equivalent compositions of motif B. Comparison between the motifs of the art and those of the invention indicate a motif A and B sequences will be functional in plants while allowing for a certain level of degeneracy as indicated in SEQ ID NO:77 (motif A) and SEQ ID NO:78 (motif B).

Split inteins, composed of an N-terminal portion (IntN) and a C-terminal portion (IntC), have been discovered naturally (e.g., the split DnaE genes of Synechocystis sp. PCC6803) and made synthetically (see Mills, K. V. Proc. Natl. Acad. Sci. USA. 95: 3543-3548 (1998); Southworth, M. W. et al. EMBO. 17:918-926 (1998); Wu, H. et al. Biochimica et Biophysica Acta 187:422-432 (1998); Yamazaki, T. et al. J. Am. Chem. Soc. 120:5591-5592 (1998)). The literature provides abundant knowledge demonstrating the critical motifs required for functional inteins. Thus, it is envisioned that a variety of mutated split inteins could be generated that would still possess the ability to self-excise from a protein precursor.

Inteins can be modified to contain optimized codons for a specific host. The present invention provides sequences for a split intein containing plant optimized codons. A split intein sequence containing optimized codons for a specific plant host can be generated by following the teachings of the present invention and techniques known in the art, such as Murray et al. (Nucl. Acids. Res. 17(2):477-498 (1989)). It is expected that once an intein system is developed in a given crop, the intein system can be easily adapted for conditional activation of a variety of target trait genes and for production of large protein polymers.

Exteins Pairs, which Yield Mature and Active Transgenic Proteins

Exteins pairs refer to an N-terminal portion of a protein precursor extein (ExtN), and a C-terminal portion of a protein precursor extein (ExtC) that are ligated together in the intein-mediated protein splicing process to yield a mature and active transgenic protein, which no longer possesses a blocking intein fragment. Exteins of the present invention will be those that convey a desirable phenotype on the transformed plant, those that produce a desirable product in the host plant, or those that may be harvested from the plant and combined in vitro to produce an active protein that otherwise could not be readily synthesized in the plant host.

Particularly desirable exteins in the present invention are those which could be useful as protein building blocks, for assembly into hybrid protein polymers. Exteins having distinct domains and functions (i.e., protein building blocks) could be spliced together by the process of the present invention, to yield large multidomain and/or multifunctional proteins or large homogeneous protein polymers, in vitro or in vivo. Each extein building block could thus represent variable “designer” specialty domains (e.g., a β-turn, a catalytic domain for a particular enzyme, etc.) or possess other special characteristics (e.g., amino acid length and structure) that could be selectively bred into plants. Subsequent crossing of the appropriate plant lines, each containing a desired extein building block, would yield a protein polymer with the predesigned functionalities and/or molecular size. Thus, particularly useful transgenes will include, but not be limited to: genes which encode for strong structural proteins such as silk, collagen, and elastin; or, those genes with special functional domains such as a cellulose or metal -binding domains. It is also suggested that plant-produced peptide building blocks could also be ligated with other types of natural or synthetic building blocks mediated by inteins, after isolation from plant hosts.

Exteins can encode other foreign proteins not natively produced in the plant hosts. Such foreign proteins will include, for example, enzymes for primary or secondary metabolism in plants, proteins that confer disease or herbicide resistance, commercially useful non-plant enzymes, and proteins with desired properties useful in animal feed or human food. Additionally, foreign proteins encoded by the transgenes will include seed storage proteins with improved nutritional properties, such as the high-sulfur 10 kD corn seed protein or high-sulfur zein proteins. Additional examples of a transgene suitable for use in the present invention include genes for disease resistance (e.g., gene for endotoxin of Bacillus thuringiensis, WO 92/20802)), herbicide resistance (mutant acetolactate synthase gene, WO 92/08794)), seed storage protein (e.g., glutelin gene, WO 93/18643)), fatty acid synthesis (e.g., acyl-ACP thioesterase gene, WO 92/20236)), cell wall hydrolysis (e.g., polygalacturonase gene (D. Grierson et al., Nucl. Acids Res., 14: 8595 (1986)), anthocyanin biosynthesis (e.g., chalcone synthase gene (H. J. Reif et al., Mol. Gen. Genet., 199: 208 (1985)), ethylene biosynthesis (e.g., ACC oxidase gene (A. Slater et al., Plant Mol. Biol., 5: 137 (1985)), active oxygeN-scavenging system (e.g., glutathione reductase gene (S. Greer & R. N. Perham, Biochemistry, 25: 2736 (1986)), and lignin biosynthesis (e.g., phenylalanine ammonia-lyase gene, cinnamyl alcohol dehydrogenase gene, o-methyltransferase gene, cinnamate 4-hydroxylase gene, 4-coumarate-CoA ligase gene, cinnamoyl CoA reductase gene (A. M. Boudet et al., New Phytol., 129: 203 (1995)).

Exteins may also be chosen, such that upon intein-mediated protein splicing in plants, a circular recombinant protein or enzymes with higher stability are produced. Trans-splicing activity of the Ssp DnaE intein has been successfully applied to the cyclization of a protein in vivo in bacteria (Evans, T. C. et al. J. Biol. Chem. 275:9091-9094 (2000); Scott, C. P. et al. P.N.A.S. 96: 13638-13643 (1999)). It is known that circularized polymers may possess special properties that are not found in the comparable linearized molecule, and thus the ability to create a circularized polymer in vivo in a plant host could be especially useful.

Exteins may also function as transformation markers. Transformation markers include: selectable genes (e.g., antibiotic or herbicide resistance genes), which are used to select transformed cells in tissue culture; non-destructive screenable reporters (e.g., green fluorescent and luciferase genes); or, a morphological marker (e.g., such as “shooty”, “rooty”, or “tumorous” phenotype).

Additionally, exteins may encode proteins that affect plant morphology and thus may also be used as markers. Morphological transformation marker genes include cytokinin biosynthetic genes, such as the bacterial gene encoding isopentenyl transferase (IPT; Ebumina et al. Proc. Natl. Acad. Sci. USA 94:2117-2121 (1997); and Kunkel et al. Nat. Biotechnol. 17(9): 916-919 (1999)). Other morphological markers include developmental genes that can induce ectopic shoots, such as Arabidopsis STM, KNAT 1, AINTEGUMANTA, Lec 1, Brassica “Babyboom” gene, rice OSH1 gene, or maize Knotted (Kn1) genes. Yet other morphological markers are the wild type T-DNA of Ti and Ri plasmids of Agrobacterium that induce tumors or hairy roots, respectively, or their constituent T-DNA genes for distinct morphological phenotypes, such as shooty (e.g., cytokinin biosynthesis gene) or rooty phenotype (e.g. rol C gene).

Plant Hosts and Transformation Methods

The present invention additionally provides plant hosts for transformation with the present intein cassettes. Moreover, the host plants for use in the present invention are not particularly limited. Examples of useful host plants are categorized as food plants (annuals), non-food plants (annuals), arboreous plants, and aquatic plants. Specific examples for each type of useful host plant are listed below.

Food plants (annuals): asparagus (Asparagus), banana (Musa), barley (Hordeum), blueberry (Vaccinium), broad bean (Vicia), cacao (Theobroma), capsicum pepper (Capsicum), carrot (Daucus), cassava (Manihot), corn (Zea), cucumber (Cucumis), eggplant (Solanum), Lentil (lens), lettuce (Lactuca), mango (Mangifera), oilseed rape, canola, cabbage, broccoli, cauliflower (Brassica), oat (Avena), onions (Allium), papaya (Carica), peas (Pisum), peanut (Arachis), pineapple (Ananas), pinto bean, mung bean, lima bean (Phaseolus), potato (Solanum), pumpkin, zucchini (Cucurbita), radish (Raphanus), rice (Oryza), rye (Secale), sesame (Sesame), spinach (Spinaceae), sorphum (Sorphum), soybean (Glycine), strawberry (Fragaria), sugarcane (Saccharum), sugar beet (Beta), sunflower (Helianthus), sweet potato (Ipomoea), tomato (Lycopersicom), watermelon (Citrullus), wheat (Triticum), and yam (Dioscorea).

Non-food plants (annuals): alfalfa (Medicago), amaranth (Amaranthus), angelica (Agelica), arabidopsis (Arabidopsis), castorbean (Ricinus), cotton (Gossypium), colewort (Crambe), dandelion (Taraxacum), flax (Linum), hemp (Cannabis), jojoba (Simmondsia), jute (Corchorus), kenaf (Hibiscus), lupine (Lupinus), petunia (Petunia), plantain (Plantago), sisal (Agave), snapdragon (Antirrhinum), switch grass (Panicum), and tobacco (Nicotiana).

Arboreous plants: apple (Malus), acacia (Acacia), chestnut (Castanea), citrus (Citrus), coconut (Cocos), coffee (Coffea), cypress (Cupressus), eucalypti (Eucalyptus), grape (Vitis), hemlock (Tsuga), hickory (Carya), maple (Acer), oak (Quercus), pear (Pyrus), peach, plum, cherry (Prunus), pine (Pinus), poplar (Populus), rose (Rosa), spruce (Picea), and walnut (Juglans).

Aquatic plants: brown alga (Laminaria), duckweed (Lemna), green alga (Chlamydomonas), and red alga (Porphyra).

However, the host plants for use in the present invention are not limited thereto.

One skilled in the art recognizes that the expression level and regulation of a transgene in a plant can vary significantly from line to line. Thus, one has to test several lines to find one with the desired expression level and regulation. Once a line is identified with the desired regulation specificity for a particular split intein cassette, it can be crossed with lines carrying different split intein cassettes for production of a mature active protein from each individual N- and C-polypeptide.

A variety of techniques are available and known to those skilled in the art for introduction of constructs into a plant cell host. These techniques include transformation with DNA employing A. tumefaciens or A. rhizogenes as the transforming agent, particle acceleration, electroporation, etc. (See for example, EP 295959 and EP 138341). It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti et al., Bio/Technology 3:241 (1985); Byrne et al., Plant Cell, Tissue and Organ Culture 8:3 (1987); Sukhapinda et al., Plant Mol. Biol. 8:209-216 (1987); Lorz et al., Mol. Gen. Genet. 199:178 (1985); Potrykus Mol. Gen. Genet. 199:183 (1985); Park et al., J. Plant Biol. 38(4): 365-71 (1995); Hiei et al., Plant J. 6:271-282 (1994)). The use of T-DNA to transform plant cells has received extensive study and is amply described (“Arabidopsis protocols”. In Methods in Molecular Biology Vol. 82; Martinez-Zapater, J. M., and Salinas, J., Eds.; Humana: Totowa, N.J., 1998; Plant Molecular Biology, A Laboratory Manual, Clark, M. S., Ed. Springer-Verlag: Berlin, Heidelberg, 1997; and Methods in Plant Molecular Biology, A Laboratory Course Manual, Maliga, P., et al., Eds; Cold Spring Harbor Laboratory: Cold Spring Harbor, N.Y., 1995). For introduction into plants, the intein cassettes of the invention can be inserted into binary vectors as described in the examples.

Other transformation methods are available to those skilled in the art, such as high-velocity ballistic bombardment with metal particles coated with the nucleic acid constructs (see Kline et al., Nature (London) 327:70 (1987), and see U.S. Pat. No. 4,945,050), direct uptake of foreign DNA constructs (see EP 295959), or techniques of electroporation (see Fromm et al., Nature (London) 319:791 (1986)). Once transformed, the cells can be regenerated by those skilled in the art. Of particular relevance are the recently described methods to transform foreign genes into commercially important crops, such as rapeseed (see De Block et al., Plant Physiol. 91:694-701 (1989)), sunflower (Everett et al., Bio/Technology 5:1201 (1987)), soybean (McCabe et al., Bio/Technology 6:923 (1988); Hinchee et al., Bio/Technology 6:915 (1988); Chee et al., Plant Physiol. 91:1212-1218 (1989); Christou et al., Proc. Natl. Acad. Sci USA 86:7500-7504 (1989); EP 301749), rice (Hiei et al., Plant J., 6:271-282 (1994)), and corn (Gordon-Kamm et al., Plant Cell 2:603-618 (1990); Fromm et al., Biotechnology 8:833-839 (1990)).

Transgenic plant cells are then placed in an appropriate selective medium for selection of transgenic cells that are then grown to callus. Shoots are grown from callus and plantlets generated from the shoot by growing in rooting medium. The various cassettes normally will be joined to a marker for selection in plant cells. Conveniently, the marker may be resistance to a biocide (particularly an antibiotic such as kanamycin, G418, bleomycin, hygromycin, chloramphenicol, herbicide, or the like). The particular marker used will allow for selection of transformed cells as compared to cells lacking the DNA that has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region which is not native to the gene from which the transcription-initiation-region is derived.

To confirm the presence of the transgenes in transgenic cells and plants, a Southern blot analysis or PCR can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts may be harvested, and/or the seed collected. The seed may serve as a source for growing additional plants with tissues or parts having the desired characteristics.

Applications of Intein-Mediated Protein Splicing in Plants

The present invention provides a method in plant gene expression that enables use of inteins to autonomously produce an active protein (ExtN-ExtC) in the plant by ligation of flanking exteins, ExtN and ExtC. The technology demonstrated in the present application is particularly useful as it proves that known bacterial inteins, such as the split Ssp DnaE inteins, function effectively in plants when the genes are modified to contain plant optimized codons. Applications of this technique permits the conditional or regulated expression of transgenes in higher plants under selected environmental conditions, in selected plant tissues, at selected developmental stages, or in selected plant generations.

The constructs of the invention are referred to as intein cassettes. Each intein cassette will comprise at least one intein or portion thereof containing plant optimized codons and one extein. Various regulatory sequences, intervening blocking sequences, and other DNA may be located within the intein cassette. Regulatory sequences may include constitutive, inducible, tissue specific or developmental stage-specific promoters, 3′ terminator sequences, and other regulatory elements. One N-nucleotide sequence will typically include a single promoter operably linked to the split intein IntN fragment and ExtN. A C-nucleotide sequence will typically include a promoter that drives the expression of IntC and ExtC. Transgenes of the present invention will encode hybrid proteins, complex polymers, genetic traits, or various transformation or morphological markers. Only by configuring the intein cassettes and placing them carefully to enable intein-mediated protein splicing of ExtN and ExtC will production of an active protein be permitted within the plant. This permits placement of intein cassettes in different parental plants or the same parental plant. The result of this invention is active expression of the transgene under selected environmental conditions, in selected plant tissues, at selected developmental stages, or in selected plant generations. It will be appreciated that any number of intein cassettes may be created with these essential components to permit expression of any number of transgenes.

Application of this intein-mediated protein splicing technology in plants lends itself well to applications requiring protein assembly. Specifically, the intein catalyzes its own removal from a protein precursor and ligates the flanking peptide sequences ExtN and ExtC to produce a mature active protein (ExtN-ExtC). In trans-protein splicing, a pair of split inteins assemble two separate, disassociated peptides into a mature and active protein. The reaction is mediated entirely by the intein, while the particular extein sequence has no limitation. Based on the ability of inteins to function both in vitro and in vivo in all plant tissues, and on split inteins' ability to effectively function on either two separate loci or within the same locus, applications of these techniques in plant genetic engineering are further extended.

One embodiment of the invention is for assembly of recombinant protein or protein-derived products. Plants, like many other organisms, can only synthesize recombinant proteins efficiently within a certain molecular weight range. For example, plants can efficiently synthesize high quality 65 kD silk-like protein (SLP); however, SLP larger than 125 kD is produced with significantly lower efficiency and diminished quality. This difficulty could be overcome using the intein-mediated protein splicing mechanism, as each 65 kD SLP precursor could be readily synthesized without stressing the plant's native protein synthesis machinery. Then, the protein precursors could be subsequently assembled via the intein-mediated ligation process in vivo, to produce a 125 kD SLP representing a “large homogeneous SLP polymer”. This strategy would enable plants to overcome their natural limitations concerning protein synthesis and thereby synthesize high molecular weight protein polymers.

A further embodiment of the invention requires combination of the protein splicing technology herein with plant breeding or in vitro splicing techniques. For example, advanced SLP polymers often require additional functionalities, created by adding selected functional domains to a basic SLP sequence (O'Brien, J. P., et al., Advanced Materials 10:1185-1195 (1998)). To make this kind of “hybrid molecule”, a SLP sequence could be fused to IntN and transformed into a N-plant host, while the selected functional domain could be fused with IntC and transformed into a C-plant host. This process could be repeated, to create a suite of N- and C-plant hosts which each host containing desired peptide building blocks, in the form of various N- and C-nucleotide sequences present within each plant. SLP trait and functional domain traits could be crossed selectively, according to the demands of the breeding program, to produce special advanced SLP polymers in the progeny plants via in vivo intein-mediated assembly. If one peptide building block was SLP and another building block was a functional domain, the final product produced in the progeny plants would be a “hybrid SLP molecule”. If both peptide building blocks were SLPs, the final product produced in the progeny plants would be a “large homogeneous SLP molecule”. In comparison to traditional methods, based on the “one gene for one protein” model, this production platform provides much greater efficiency and flexibility.

As ExtN and ExtC, SLPs and other peptide building blocks could also be individually produced and isolated from their respective host plant. Then, subsequent assembly of the mature protein as a “hybrid SLP polymer” or “large homogeneous SLP polymer” could be performed in vitro. The significant advantage associated with in vitro assembly is the ability to use building blocks that are not peptides. Thus, other polymers (including synthetic polymers) that could be chemically linked with an intein peptide could be assembled via intein-mediated protein splicing. This could provide SLPs with a wide range of functionalities that previously have not been possible to create.

Another embodiment of the present invention is for production of toxic proteins and enzymes, using the protein splicing mechanism. Although plants are considered as low cost, high efficiency protein production platforms, many important recombinant proteins and enzymes can not be produced in plants at commercially significant levels due to incompatibility with the plant system. This may be based upon the desired protein's own incompatibility with the plant or incompatibility resulting from related pathways necessary for the transgenic protein's production. If the protein could be split genetically, fused to split inteins, and transformed into distinct N- and C-host plants, non-toxic “half-proteins” could be over-expressed and isolated from their host plants. The toxic protein or enzyme could then be produced in vitro by the intein-mediated protein splicing, according to the principles described above.

An additional embodiment of the invention, requiring the integration of intein-mediated protein splicing technology and plant genetic engineering platform technologies, is for development of sophisticated molecular switches in plant cells. One example of a molecular switch exists with respect to division of an active protein into two extein fragments. When the protein exists as two exteins, its activity is “off”. In contrast, protein activity is “on” following the intein splicing reaction and the synthesis of the intact protein. Thus, manipulation of intein-mediated protein splicing would enable the activity of a protein or enzyme to be controlled precisely, thereby enabling regulation of gene expression mechanisms, metabolism pathways, and the transgenes' impact on plant growth and the environment.

Current scientific understanding of the intein-mediated protein splicing process does not permit direct control of the intein reaction. However, several indirect methods are available, as described in the present application. First, it is possible to control intein-mediated protein splicing through the use of traditional plant breeding. This technique allows separation of intein cassettes into two distinct host plants. In this manner, the N-plant host contains the N-nucleotide sequence (containing IntN and ExtN) and the C-plant host contains the C-nucleotide sequence (containing IntC and ExtC). Protein activity is necessarily “off”. Only when these two host plants are crossed will the activity of the protein or enzyme (ExtN-ExtC) be turned “on” in the hybrid progeny, as a result of intein splicing. The activation may not be heritable in a large portion of the progeny in subsequent generations because of genetic segregation. As a result, conditional expression of the transgene and its potential activation of a central pathway in the plant progeny can be achieved in the desired T1 generation, and not in subsequent generations in large part. Benefits of this type of control of the transgenic trait include protection of manufacturers' rights in relation to hybrid seed protection and prevention of uncontrolled spread of the transgene.

Trans-protein splicing techniques also permit control of a transgene's activation by co-transformation methods. A plant host containing either a N- or C-nucleotide sequence could be subsequently transformed with the opposing N- or C-nucleotide sequence necessary in order to have a complete intein present in the plant tissue, thereby activating the transgene and turning expression of the active protein “on”.

An obvious improvement to one skilled in the art for this type of “molecular switch” strategy incorporates judicious use of promoters to control expression of each particular intein cassette present in the N- and C-plant host. After crossing or co-transformation, transcription and translation of each cassette yields an inactive N- and C-polypeptide precursor according to activation of the promoter driving expression of the N- and C-nucleotide sequence. If the promoters are “offset”, e.g. such that one polypeptide is produced over a long period of the plant's life, while the second promoter produces the second polypeptide precursor for only a short time in a specific stage of plant development, active protein will not be synthesized in the plant cell until both the N- and the C-polypeptide precursors co-exist in the plant for some period. Only when the N- and the C-polypeptide precursors co-exist can intein-mediated protein splicing and production of a mature, active protein occur. Thus, one utility envisioned is for activation of a transgene, whose expression is detrimental to normal plant development only in the first generation. Such transgenes include those that result in production of a desired product at levels that would be considered phytotoxic if expressed during breeding but that do not interfere with the plant when produced in the harvestable generation. Or, the method could serve to control the spread of transgenes via cross-pollination. As suggested by a group at New England Biolabs (Chen, L. et al. Gene 263:3948 (2001); Sun, L. et al. Appl. Envir. Micro. 67:1025-1029 (2001)), the ExtN and ExtC could be separately located on nuclear and chloroplast genomes, but reassembled to create a functional protein via intein-mediated protein splicing in the cytosol. Careful choice of the promoter controlling each intein cassette can permit a specific transgene to be expressed only under selected environmental conditions, in selected plant tissues, at selected developmental stages, or in selected plant generations. A further embodiment of the invention could incorporate added levels of control of the transgene's activation by use of site specific recombination systems (Yadav, PCT Int. Appl. WO 01/36595 A2 (2001)).

Another preferred embodiment of the invention applies understanding of the intein splicing mechanism to produce circularized proteins and/or enzymes. It has been demonstrated that split inteins are able to cyclize linear proteins, when IntN and IntC are fused to both ends of a linear protein, respectively (Evans, T. C. et al. J. Biol. Chem. 275(13): 9091-9094 (2000)). By a similar approach, the transgenic plant should be able to produce circular recombinant proteins and enzymes. Typically, a circular enzyme is usually more stable, and thus more active, than a linear enzyme. Additionally, circularized structural proteins may provide new functionality that did not exist in the corresponding linear analog.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory: Cold Spring Harbor, N.Y. (1989) (Maniatis); by T. J. Silhavy, M. L. Bennan, and L. W. Enquist, Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, Eds., American Society for Microbiology: Washington, D.C. (1994)) or by Thomas D. Brock in Biotechnology: A Textbook of Industrial Microbiology, 2nd ed., Sinauer Associates: Sunderland, Mass. (1989). All reagents, restriction enzymes and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Manipulations of genetic sequences were accomplished using the suite of programs available from the Genetics Computer Group Inc. (Wisconsin Package Version 9.0, Genetics Computer Group (GCG), Madison, Wis.). Where the GCG program “Pileup” was used, the gap creation default value of 12 and the gap extension default value of 4 were used. Where the GCG “Gap” or “Bestfit” programs were used, the default gap creation penalty of 50 and the default gap extension penalty of 3 were used. In any case where GCG program parameters were not prompted for, in these or any other GCG program, default values were used.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” mean micromole(s)”, “g” means gram(s), “μg” means microgram(s), “ng” means nanogram(s), “U” means unit(s), “bp” means base pair(s), “kB” means kilobase(s), and “kD” means kilodalton(s).

### Example I

### Synthesis and Assembly of DNA Sequences Encoding Ssp DnaE Intein

Example 1 describes the method used to alter the native amino acid sequence of the DnaE split intein of Synechocystis sp. PCC6803 such that it contained plant-optimized codons suitable for expression of the split intein in a plant host.

The naturally split DnaE intein identified in Synechocystis sp. PCC6803 mediates a protein trans-splicing reaction to produce a mature catalytic subunit of DNA polymerase III. The native peptide sequences of the DnaE split intein are shown in Table 1.

TABLE 1

Peptide Sequences of the split intein DnaE from
Synechocystis sp. PCC6803

| | | | |
|---|---|---|---|
| | | | SEQ |
| | Length | | ID |
| Intein | (AA) | Sequence* | NO |

| | | | |
|---|---|---|---|
| Int-n | 123 | CLSFGTEILTVEYGPLPIGKIVSEEINCSVYS | 1 |
| | | VDPEGRVYTQAIAQWHDRGEQEVLEYELE | |
| | | DGSVIRATSDHRFLTTDYQLLAIEEIFARQLD | |
| | | LLTLENIKQTEEALDNH RLPFPLLDAGTIK | |
| Int-c | 36 | MVKVIGRRSLGVQRIFDIGLPQDHNFLLANG | 2 |
| | | AIAAN_{———}(C) | |

*All four conserved motifs are underlined. Amino acid residues required for protein splicing are shown in bold text. A cysteine immediately downstream of Int-c (shown in parentheses) is also required for protein splicing.

To utilize this split intein in transgenic plants, synthetic genes of the split intein were synthesized and assembled, to contain plant optimized codons.

To synthesize the 123 amino acid IntN and 36 amino acid IntC sequences, a series of nucleotide oligomers were designed, that would allow generation of a series of overlapping DNA fragments which could then subsequently be assembled and amplified by PCR into a complete IntN and IntC gene. At first, four groups of nucleotide oligomers were designed according to the peptide sequences of DnaE split intein Synechocystis sp. PCC6803 (Wu, H. et al. Proc. Natl. Acad. Sci. USA. 95:9226-9231 (1998)) and using the rules of genetic codon usage in plants (Murray, E. E., et al. Nucl. Acids. Res. 17:477498 (1989)). These oligomers, categorized into 5 different groups, are presented below in Table 2.

TABLE 2
Oligomers for Synthesis of the split intein
DnaE from Synechocystis sp. PCC6803

| | | Length | SEQ ID |
|---|---|---|---|
| Group | Name | (bp) | NO |
| 1 | IntN + 1 | 75 | 3 |
| 1 | IntN + 2 | 75 | 4 |
| 1 | IntN + 3 | 75 | 5 |
| 1 | IntN + 4 | 75 | 6 |
| 1 | IntN + 5 | 69 | 7 |
| 2 | IntN − 1 | 30 | 8 |
| 2 | IntN − 2 | 75 | 9 |
| 2 | IntN − 3 | 75 | 10 |
| 2 | IntN − 4 | 75 | 11 |
| 2 | IntN − 5 | 75 | 12 |
| 2 | IntN − 6 | 39 | 13 |
| 3 | IntC + 1 | 75 | 14 |
| 3 | IntC + 2 | 36 | 15 |
| 4 | IntC − 1 | 75 | 16 |
| 4 | IntC − 2 | 36 | 17 |
| 5 | Int − nN | 21 | 18 |
| 5 | Int − nC | 24 | 19 |
| 5 | Int − cN | 24 | 20 |
| 5 | Int − cC | 21 | 21 |

Five oligomers in group 1 and six oligomers in group 2 were complemented and overlapped with one another. Group 1 oligomers could be assembled to create the sense strand encoding Ssp DnaE Int-n, while group 2 oligomers assemble to create the antisense strand. Together, these two synthesized fragments yielded a double-stranded DNA sequence incoding Ssp DnaE Int-n, named as PInt-n (nucleotide sequence presented as SEQ ID NO:22; amino acid sequence presented as SEQ ID NO:23). Smilarly, two oligomers in group 3 and two oligomers in group 4 were also complemented and overlapped with one another, leading to assembly of a DNA fragment encoding Ssp DnaE Int-c with an additional C-terminal codon of cysteine. The DNA fragment was designed as PInt-c (nucleotide sequence presented as SEQ ID NO:24; amino acid sequence presented as SEQ ID NO:25).

To assemble the DNA fragments, all oligomers in one group were pooled into a 100 μL phosphorylation reaction, which contained 200 pmole of each oligomer, 0.1 mM ATP, 20 units T4 polynucleotide kinase (Life Technologies, Rockville, Md.), and 1×forward reaction buffer (Life Technologies). After a 0.5-hr incubation at 37° C., the reaction was stopped and cleaned up using a Qiaquick Nucleotide Removal Kit (QIAGEN, Valencia, Calif.). The phosphorylated oligomers from groups 1 and 2 were then mixed and subjected to an annealing program on a GeneAmp PCR System 9600 (Perkin Elmer, Norwalk, Conn.), which included heating at 98°C. for 10 min followed by a 75° C. temperature drop at a slope of 1° C. per 5 min. The oligomers from groups 3 and 4 were mixed and subjected to the same annealing program. Finally, the annealed oligomers were ligated at 16° C. overnight in a 100 μL reaction containing 2 units of T4 DNA ligase (Life Technologies) and 1×ligase reaction buffer. The reactions were cleaned up using QIAquick PCR Purification Kits (QIAGEN,).

To amplify the correctly assembled DNA fragments, oligomers from Group 5 (SEQ ID Nos: 18-21) were additionally synthesized and used as primers in two 50 PL-PCR reactions. The reactions contained 0.25 mM of each dNTP, 2.5 units Pfu DNA polymerase (STRATAGENE, La Jolla, Calif.), and 1×Pfu buffer. In addition, one reaction included 25 pmole of oligomer Int-nN and Int-nC as primers (SEQ ID NOs: 18 and 19, respectively) and 2 μL of PInt-n assembly reaction as template, while another included 25 nmole of oligomer Int-cN and Int-cC as primers (SEQ ID NOs: 20 and 21, respectively) and 2 μL of PInt-c assembly reaction as template. The reactions were carried out on a GeneAmp PCR System 9600 for 35 cycles by following a program of denaturation at 94° C. (45 sec), annealing at 60° C. (45 sec), and 1 min amplification at 72° C. Oligomer Int-nN and oligomer Int-nC amplified fragment PInt-n and added a stop codon at its 3′ end. Oligomer Int-cN and oligomer Int-cC amplified fragment PInt-C and created a Ncol site at its 5′ end.

Both PCR reactions were subjected to denatured agarose gel electrophoresis, gel isolation, and purification using a QIAquick Gel Extract Kit (QIAGEN). These PInt-n and PInt-c fragments were subcloned into pPCR-Script Amp plasmids, according to the manufacturer's instructions (PCR-Script Cloning Kit, STRATAGENE), resulting in new plasmids pPInt-n and pPInt-c. Plasmid DNA was then generated and isolated from XL10-Gold E. coli cells (STRATAGENE) by using a QIAprep Miniprep Kit (QIAGEN). Plasmids were subjected to sequencing to confirm correct synthesis of PInt-n and PInt-c fragments.

### Example 2

### Modification of the GUS Reporter Gene

In this example, the GUS reporter gene encoding a β-glucuronide was chosen as a model extein, as it was rather large in size (68 kD) and its functionality could be tested visually by its color reaction when the protein was active (i.e., properly spliced and folded). This gene was artificially “split” into 2 portions, representing ExtN and ExtC, and each extein was engineered to possess a 6×His tag, to facilitate subsequent isolation and detection of each extein.

An intact GUS gene encodes for a 68 kD δ-glucuronidase (E.C.3.2.1.31), which catalyses the hydrolysis of a wide variety of glucuronides. This gene was chosen as representative of many large proteins that would be desirable to express in a plant via intein-mediated protein splicing. The reporter gene is also accepted in the art as a practical model system, as the enzyme is larger than other known reporter enzymes (such as GFP) and its functionality could be tested visually by its color reaction when the protein was active (i.e., properly spliced and folded). It is expected that a host of other transgenes could be used with the present technology.

To modify the GUS gene, PCR oligomers HGUSH-n and GUSC-Bam were synthesized (SEQ ID NOs: 26 and 27). Oligomer HGUSH-n introduced a coding sequence for peptide MAHHHHHH (SEQ ID NO:63) at the N-terminus of GUS, while oligomer GUS-C-Bam added a BamHI site right after the stop codon of GUS.

GUS was amplified from plasmid pML63, provided by DuPont Agricultural Products (Wilmington Del., 19898). Vector pML63 contains the uidA gene (which encodes the GUS enzyme) operably linked to a 5′ CaMV 35S/Cab22L promoter and a 3′ NOS terminator sequence (35S/Cab22L Pro::GUS::NOS Ter). pML63 was derived from pMH40 (described in WO 98/16650) by replacing the 770 base pair terminator sequence contained in pMH40 with a new 3′ NOS terminator sequence comprising nucleotides 1277 to 1556 of the sequence published by Depicker et al. (J. Appl. Genet. 1:561-574 (1982)).

A 50-μL PCR mixture was prepared, including 20 pmoles of each oligomer, 100 ng GUS-containing pML63 plasmid, 0.25 μM each of dNTP, 2.5 units pfu polymerase, and 1×pfu buffer. The reaction was carried out on a GeneAmp PCR System 9600 for 35 cycles, following a program of denaturation at 94° C. (45 sec), annealing at 58° C. (45 sec), and amplification at 72° C. (90 sec). The product HGUS was gel-purified using a QIAquick Gel Extraction Kit and subcloned into pPCR-Script Amp plasmids (PCR-Script Cloning Kit, STRATAGENE). The resultant plasmid was generated and isolated from XL10-Gold E. coli cells by using a QIApre Miniprep Kit. The HGUS sequence was confirmed by DNA sequencing. This resultant plasmid was further subjected to restrictive enzyme digestion with Bam HI and Ncol. The HGUS fragment was separated on an agarose gel and purified using a QIAquick Gel Extraction Kit.

Plasmid GY101 (disclosed in U.S. application Ser. No. 09/863,859) was chosen as an appropriate vector into which the GUS gene would be further modified. pGY101 is a pBluscript based plasmid, resulting from a short sequence insertion of MARSRGSHHHHHH-stop codon (SEQ ID NO:64) into Bluescript. Additionally, this sequence also introduced Ncol, BgIII, Xbal, BamHI, and EcoRi sites into the plasmid. The vector was linearized with BamHi and Ncol and purified for cloning purposes, using similar protocols to those above for GUS. Linearization removed the majority of the short sequence insertion from pGY101, leaving only the 6×His tag plus the stop codon in the Bluescript based vector.

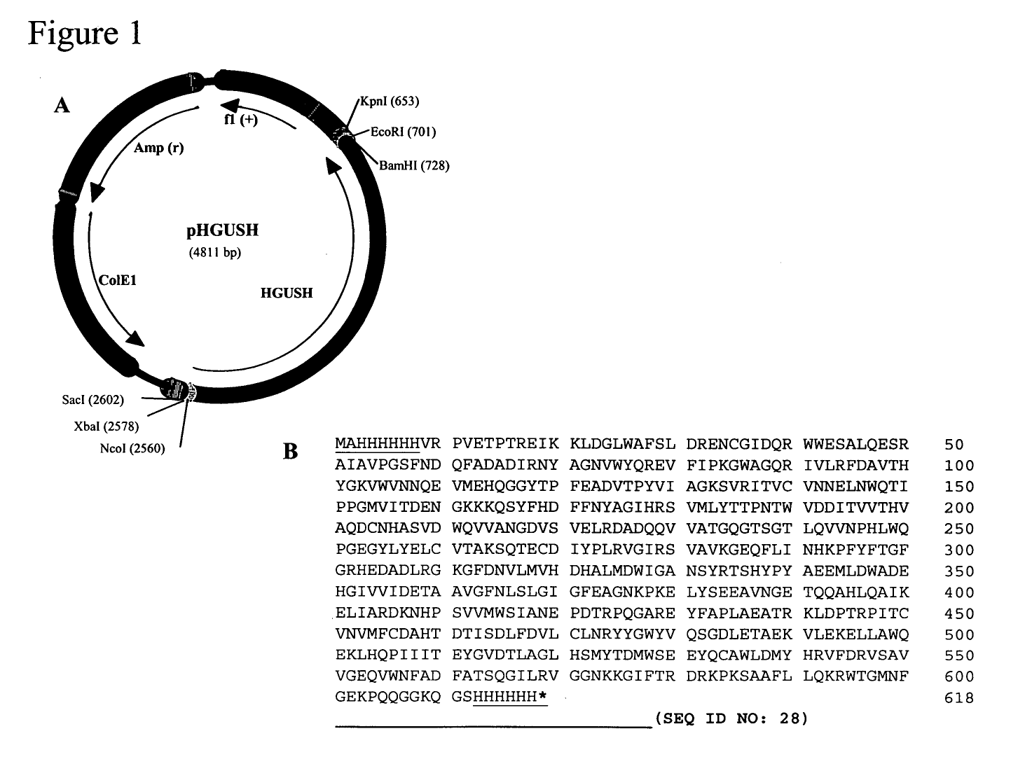

The HGUS fragment was ligated with the linearized pGY101 by T4 DNA ligase. Thus, a 6×His peptide with a stop codon was integrated with the C-terminus of the HGUS fragment in the resultant plasmid, named pHGUSH (FIG. 1A). This plasmid was generated in and isolated from XL1-Blue E. coli cells (STRATAGENE). The HGUSH region in pHGUH, encoding a GUS protein with 6×His tags at both N- and C-termini (FIG. 1B; SEQ ID NO:28), was confirmed by DNA sequencing using the universal primers T3 and T7 and customized primers GUS-N2 and GUS-C2 (SEQ ID NOs: 29 and 30).

### Example 3

### Construction of the Split Intein/GUS Fusions

Example 3 describes the creation of split intein-GUS fusions, to produce the two distinct intein cassettes. The first contained an N-nucleotide sequence having the generic structure P-IntN-ExtN, where P is a promoter suitable to drive the expression of IntN-ExtN, IntN is the N-terminal portion of the SspE split intein containing plant optimized codons (as generated in Example 1), and ExtN is the N-terminal portion of GUS (as generated in Example 2). Likewise, a C-nucleotide sequence containing the generic structure P-IntC-ExtC was created.

Creation of an in-frame fusion of GUS-n/Ssp DnaE Int-n and Ssp DnaE Int-c/GUS-c was necessary to examine the intein-mediated protein trans-splicing reaction in plants. In order to avoid adding unnecessary sequences at the junctions between inteins and GUS fragments, however, a PCR-directed recombination-mediated plasmid construct technique was applied to make the fusions. This strategy was in contrast to that used in other studies (Chen, L. et al. Gene. 263:39-48 (2001); Sun, L. et al. Appl. Envir. Micro. 67:1025-1029 (2001)). Specifically, the design of the intein-GUS fusions herein did not utilize insulating linker peptides between each intein fragment and extein fragment ((5-10 amino acids, optionally derived from Ssp DnaE extein fragments immediately flanking the inteins) which may interfere with the final protein product and prevent synthesis of an intact native enzyme. Instead the split intein-GUS fusions were direct.

A DNA fragment containing the 2 μm yeast replication origin and a Trp selective marker was amplified by PCR as described in PCT WO99/22003. One PCR reaction contained 50 μL Platinum PCR SuperMix (Life Technologies, Rockville, Md.) and 10 pmoles of primer trpN-SstII (2 μM) (SEQ ID NO:31) and primer trpC-Sstil (2 μM) (SEQ ID NO:32).

Amplification was carried out on a GeneAmp PCR System 9600 for 35 cycles, following a program of denaturation at 94° C. (45 sec), annealing at 55° C. (45 sec), and amplification at 72° C. (90 sec). Due to the primers' design, the fragment was flanked by two 25 bp DNA sequences, which were homologues to pBluscript SK(+) sequences surrounding the Sstll site.

The fragment was integrated into pBluescript SK(+) through a homologous recombination mechanism by co-transforming into yeast. A 350-pL transformation mixture included approximately 100 ng of the DNA fragment from the PCR reaction, 100 ng SstII linearized pBluescript SK(+), 120 μg PEG, 100 mM LiOAc, and 50 μg single strand DNA. It was mixed with 50 μL of yeast W303-1A component cells and incubated at 30° C. for 30 min and then at 42° C. for 20 min. The transformed yeast cells were grown on trp selective medium at 30° C. for 2 days, which contained 12 g glucose, 4 g Yeast Nitrogen Base without amino acids (Difco, Detroit, Mich.), 1.2 g Drop-Out Mix (SCM-TRP; Bufferad, Lake Bluff, Ill.), 12 g Bacto Agar (Difco), and 600 mL water. DNA was prepared from a collection of all colonies using EZ Yeast Plasmid Miniprep Kit (Geno Tech, St. Louis, Mo.) and transformed into XL1-Blue E. coli cells. Plasmid p2 μm-Trp was identified from the XL1-Blue transformants by specific restriction enzyme digestion, which confirmed a 2 μm-Trp DNA fragment within the polylinker of pBluescript SK(+).

To integrate the 2 μm-Trp DNA fragment in plasmid p2 μm-Trp into plasmids pPInt-n and pPInt-c, all three plasmids were subjected to Notl and Sstl digestion. The 2 μm-Trp DNA fragment and linearized pPInt-n and pPInt-c plasmids were isolated from an agarose gel and purified using QIAquick Gel Extraction Kits. The 2 μm-Trp DNA fragments were then subcloned into either pPInt-n or pPInt-c in ligation reactions. The resultant plasmids were identified as pPInt-N-2 μm and pPInt-C-2 μm. Their 2 μm-Trp insertions were confirmed by specific restriction enzyme digestion. Both plasmids were linearized by restriction enzyme digestion of Small and EcoRI.

Plasmid PHGUSH was digested with Xbal and EcoRI and the HGUSH fragment was isolated. Five oligomers (IntN-GusN(−) (SEQ ID NO:33); BS-GusN(+) (SEQ ID NO:34); IntN(6)-GusN(−) (SEQ ID NO:35); IntC-GusC(+) (SEQ ID NO:36); and BS(−) (SEQ ID NO:37)) were designed to carry out the PCR-directed recombination for in-frame fusion of GUS-n/Int-n and Int-c/GUS-c. Twenty-five (25) pmoles of the oligomers in various combinations were used in 50-μL PCR reactions containing 50 ng HGUSH fragment, 0.25 mM dNTP, 25 units pfu DNA polymerase, and 1×pfu reaction buffer. BS-GusN(+) and IntN-GusN(−) amplified a GUS-n fragment encoding the first 203 amino acid residues of the GUS protein, flanked by an upstream and a downstream sequence homologous to a 25-bp region in pBluescript SK(+) polylinker and the first 25 bp of the PInt-n coding region, respectively. InteN(6)-GusN(−) and BS-GusN(+) amplified a GUS-n(6) fragment, which was identical to the GUS-n fragment but the downstream flanking region was homologous to a 25-nt PInt-n region starting at its 19^{th} nucleotide (the 7^{th} codon). IntC-GusC(+) and BS(−) amplified a GUS-c fragment encoding the remaining 415 amino acid residues of the GUS protein, flanked by an upstream and a downstream sequence homologous to the last 25 bp of the PInt-c coding region and another 25 bp region in the pBluescript SK(+) polylinker, respectively.

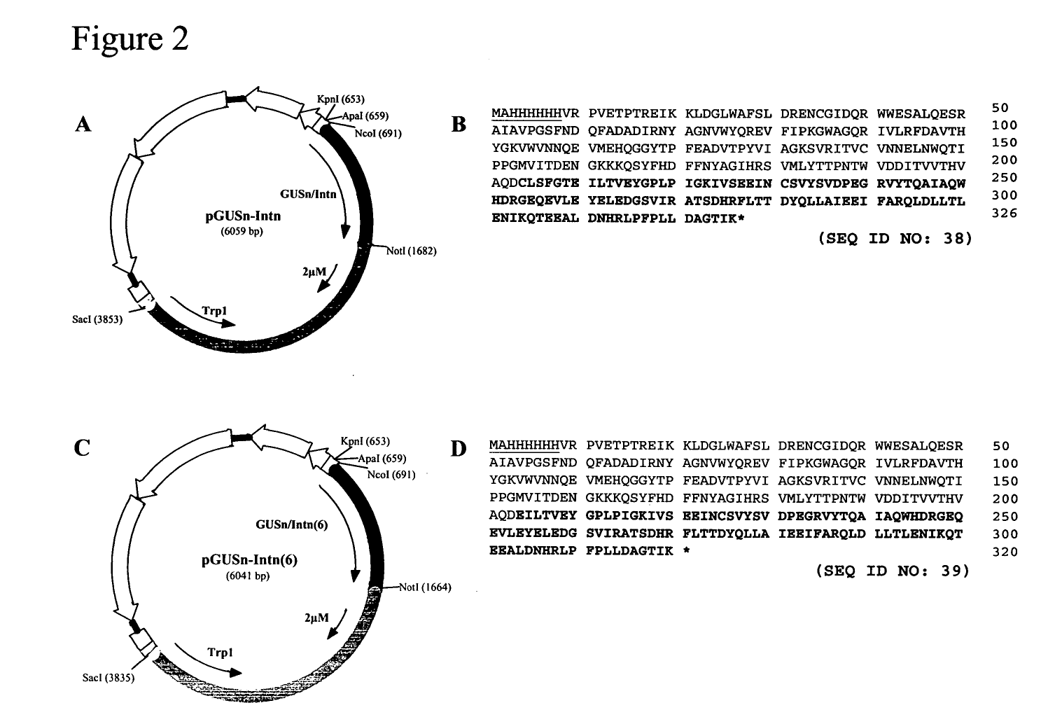

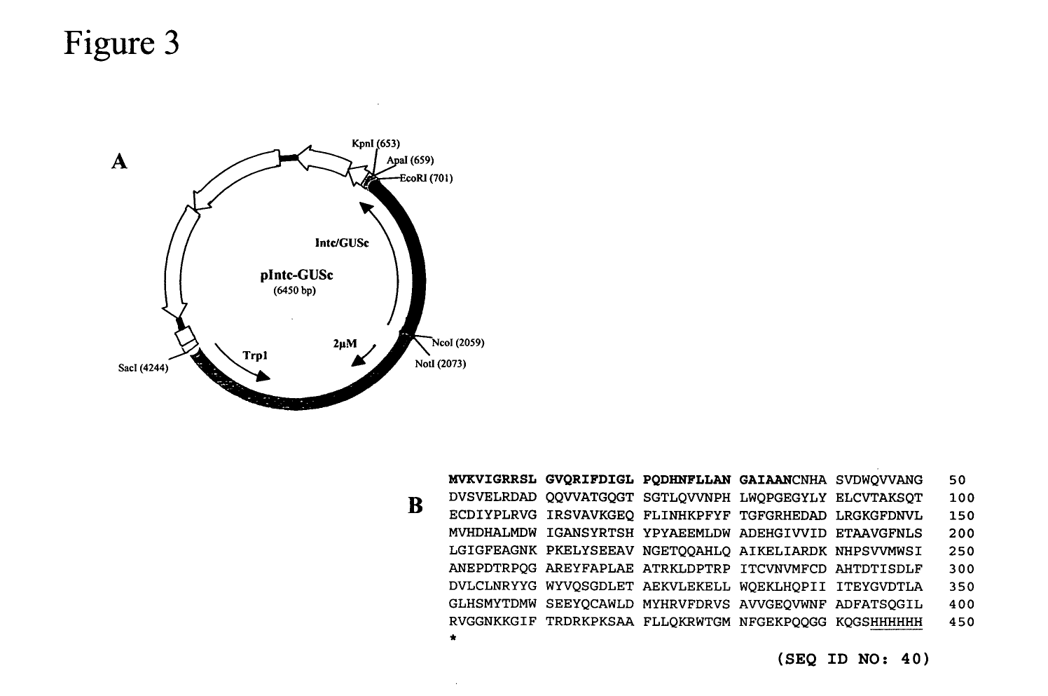

These PCR amplified DNA fragments were combined with the linearized pPInt-N-2 μm and pPInt-C-2 μm by co-transforming yeast for PCR-directed recombination-mediated construction of three fusion genes (described above). The recombination between GUS-n fragment and pPInt-N-2 μM resulted in pGUSN-Intn (FIG. 2A) containing a GUSn/Intn fusion (FIG. 2B; SEQ ID NO:38). The recombination between GUS-n(6) fragment and pPInt-N-2 μM resulted in pGUSN-Intn(6) (FIG. 2C) containing a GUSn/Intn(6) fusion (FIG. 2D; SEQ ID NO:39), where the first six amino acid residues of IntN were deleted (residues CLSFGT (SEQ ID NO:65)). The recombination between GUS-c fragment and pPInt-C-2 μM resulted in pIntC-GUSc (FIG. 3A) containing a Intc/GUSc fusion (FIG. 3B; SEQ ID NO:40). In FIG. 2B, 2D, and 3B, the His-tag is underlined in each fusion protein while the intein fragment is shown in bold text. All three new plasmids were subjected to DNA sequencing and the three fusions described above were confirmed by employing T7, IntNC, and IntCN primers.

### Example 4

### Construction of Binarv Vector-Based Expression Plasmids

The N-nucleotide sequence of GUS-n/Ssp DnaE Int-n and the C-nucleotide sequence of Ssp DnaE Int-c/GUS-c (generated in Example 3) were utilized in this example to create suitable binary vector-based expression plasmids that could be transformed into plants, and selected based on antibiotic resistance (kanamycin and glufosinate ammonium resistance).

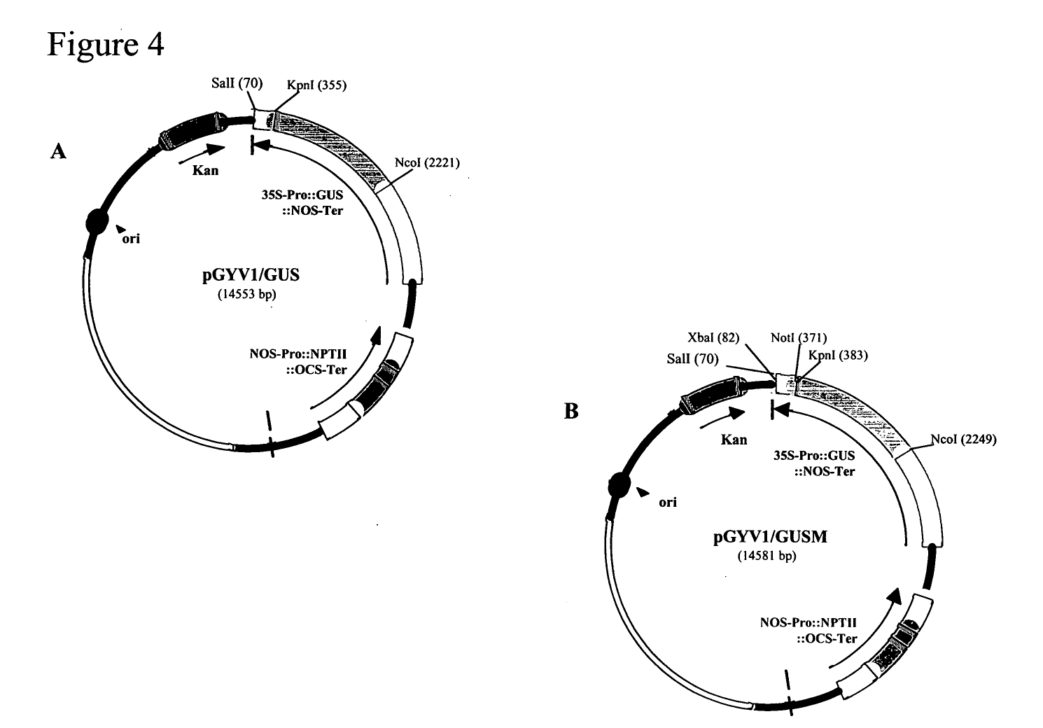

Expression plasmids were made based on pGYV1/GUS (FIG. 4A), a binary vector derived from pZBL1 (ATCC 209128; described in U.S. Pat. No. 5,968,793). When preparing pGYV1/GUS, an expression cassette of 35S-Pro::GUS::NOS-Ter was inserted into the T-DNA region of pZBL1 and many restriction sites, including a Ncol site within the NPTII gene expression cassette, were eliminated. pZBL1 includes a kanamycin resistance gene outside the T-DNA region for bacteria selection, and a NPTII gene expression cassette (NOS Pro::NPTII::OCS-Ter) inside the T-DNA region, between sequences of the right border (RB) and the left border (LB), for kanamycin resistance selection of plant cells.

Transgenes encoding Int/GUS fusions were provided by pGUSN-Intn and pGUSN-Intn(6) (Example 3). However, the transgene integration required new restriction sites in all three plasmids. To create these sites, a NOS terminator region was amplified from pML63 (described in Example 2) in a standard pfu-PCR reaction, using KNNOS and NOSXS primers (SEQ ID Nos: 41 and 42). Therefore, Kpnl and a Notl sites were attached upstream of the NOS fragment, and Xbal and Sall sites were attached downstream of the NOS fragment. The fragment was digested with Kpnl and Sall and replaced the original NOS region between these two sites on pGYV1/GUS. The modified plasmid was named pGYV1/GUSM (FIG. 4B) and confirmed by restriction enzyme digestion.

Simultaneously, pGUSN-Intn and pGUSN-Intn(6) were digested with Notl and Apal. The GUSN-Intn and GUSN-Intn(6) fusions were isolated and subcloned into pCR2.1/TopD (Invitogen) between the Notl and Apal sites. The intermediate plasmids pGUSN-IntN-M and pGUSN-Intn(6)-M were also confirmed by restriction enzyme digestion.

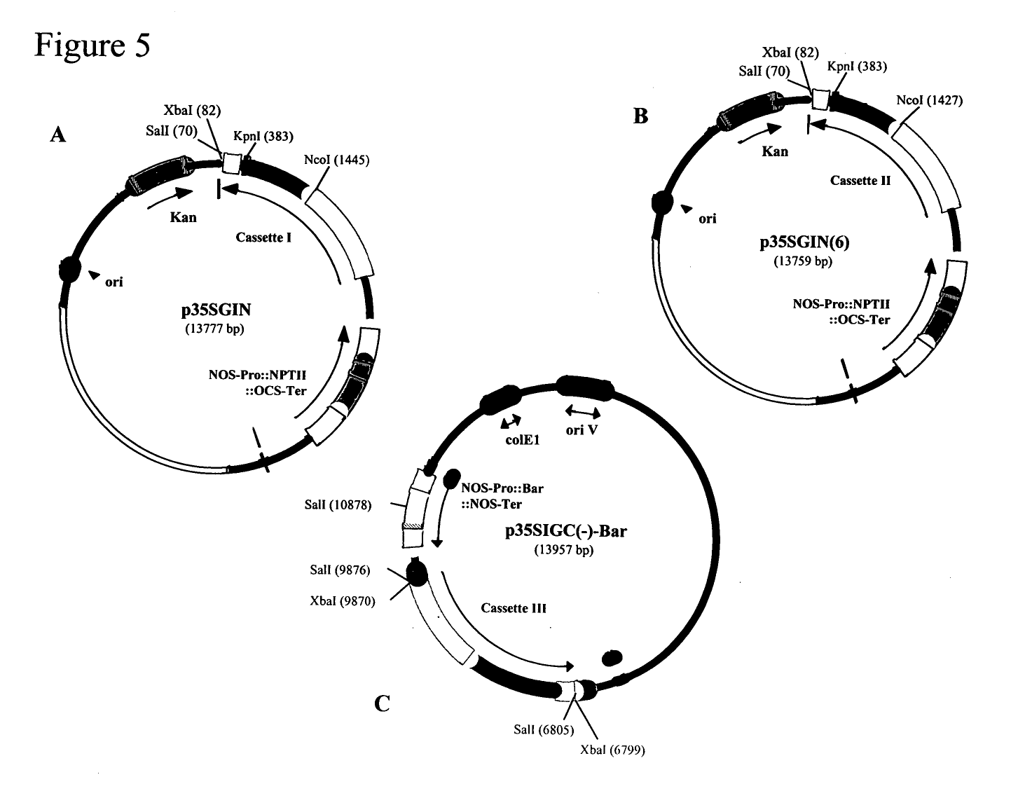

To assemble expression plasmids containing a single transgene expression cassette, pGYV1/GUSM was digested with NcoI and KpnI and the GUS coding region was removed. The remainder of pGYV1/GUSM was employed as a receptor providing a binary vector, a NPTII expression cassette for kanamycin resistance selection, and a 35S promoter-NOS terminator for transgene expression. Plasmids pGUSN-IntN-M and pGUSN-Intn(6)-M were also digested with the same enzymes. GUSN-Intn and GUSN-Intn(6) coding regions were isolated and subcloned into the above pGYV1/GUSM receptor, thus forming the binary vector-based expression plasmids p35SGIN (FIG. 5A) and p35SGIN(6) (FIG. 5B). These plasmids contained expression cassettes of 35S::GUSN-Intn::NOS and 35S::GUSN-Intn(6)::NOS, respectively, as well as the NOS Pro::NPTII::OCS-Ter expression cassette for transgenic plant selection.

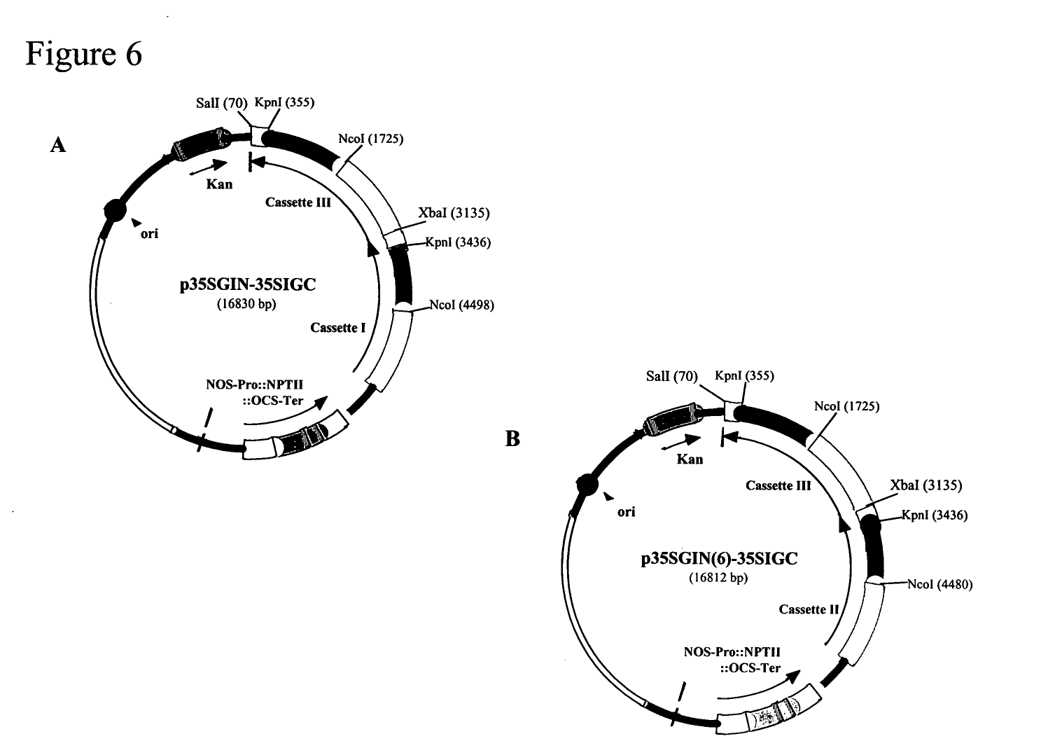

pintC-GUSc was digested with NcoI and EcoRI. The IntC-GUSc coding region isolated from the digestion was used to replace the GUS coding region in pML63 (described in Example 2). The resulting p35SIntC-GUSc had an expression cassette of 35S::IntC-GUSc::NOS. This expression cassette was isolated by Xbal digestion and inserted into the Xbal site of pBE673 (PCT WO99/22003), resulting in p35SIGC(−)-Bar (FIG. 5C). To assemble the expression plasmids containing double transgene expression cassettes, p35SGIN and p35SGIN(6) were digested with Sall and Xbal as receptors. A Sall/Xbal fragment containing 35S::IntC-GUSc::NOS was isolated from p35SIntC-GUSc and ligated into p35SGIN and p35SGIN(6) between the Sall and Xbal sites, resulting in p35SGIN-35SIGC and p35GIN(6)-35SIGC (FIG. 6A and 6B), respectively.

All intermediate and expression plasmids are summarized in Table 3, below, for easy reference.

TABLE 3
Summary of Intermediate and Expression Plasmids

| Plasmid | | | |
|---|---|---|---|
| Name | FIG. | Carrier | Purpose |
| pPInt-n | | pPCR-Script | Synthetic PInt-n fragment |
| | | Amp SK(+) | |
| pPInt-c | | pPCR-Script | Synthetic PInt-c fragment |
| | | Amp SK(+) | |
| pHGUSH | 2 | pBlucript SK(+) | HGUSH coding region |
| p2 μM-Trp | | pBlucript SK(+) | 2 μM-Trp element |
| pPInt-N-2 μM | | pPCR-Script | Attach 2 μM-Trp to pPInt-n |
| | | Amp SK(+) | |
| pPInt-C-2 μM | | pPCR-Script | Attach 2 μM-Trp to pPInt-c |
| | | Amp SK(+) | |
| pGUSN-Intn | 3A | pPCR-Script | Coding region of GUSn/Intn |
| | | Amp SK(+) | fusion |
| pGUSN-Intn( | 3C | pPCR-Script | Coding region of |
| 6) | | Amp SK(+) | GUSn/Intn(6) fusion |
| PIntC-GUSc | 4A | pPCR-Script | Coding region of Intc/GUSc |
| | | Amp SK(+) | fusion |
| pGYV1/GUS | 5A | pZBL1 | NOS::NPTII::OCS selection |
| | | | and transgene |
| | | | 35S::GUS::NOS |
| pGYV1/ | 5B | pZBL1 | NOS::NPTII::OCS selection |
| GUSM | | | and transgene |
| | | | 35S::GUS::NOS, however |
| | | | additional enzyme sites were |
| | | | created |
| pGUSN- | | pCR2.1-TopD | Additional sites flanking |
| IntN-M | | | GUSn/Intn fusion |
| pGUSN-Intn( | | pCR2.1-TopD | Additional sites flanking |
| 6)-M | | | GUSn/Intn(6) fusion |
| p35SGIN | 6A | pZBL1 | NOS::NPTII::OCS selection |
| | | | and transgene |
| | | | 35S::GUSn/Intn::NOS |
| p35SGIN(6) | 6B | pZBL1 | NOS::NPTII::OCS selection |
| | | | and transgene |
| | | | 35S::GUSn/Intn(6)::NOS |
| p35SIntC-G | | pML63 | Provides an expression |
| USc | | | cassette of |
| | | | 35S::Intc/GUSc::NOS |
| p35SIGC(−)- | 6C | pBE673 | NOS::Bar::NOS selection and |
| Bar | | | transgene |
| | | | 35S::Intc/GUSc::NOS |
| p35SGIN-35 | 7A | pZBL1 | NOS::NPTII::OCS selection |
| SIGC | | | and transgenes |
| | | | 35S::GUSn/Intn::NOS and |
| | | | 35S::Intc/GUSc::NOS |
| p35SGIN(6)- | 7B | pZBL1 | NOS::NPTII::OCS selection |
| 35SIGC | | | and transgenes |
| | | | 35S::GUSn/Intn(6)::NOS |
| | | | and 35S::Intc/GUSc::NOS |

### Example 5

### Stable Transformation of Arabidopsis Plants

This example describes the transformation of binary vector-based expression plasmids from Example 4 into Arabidoposis, a model system for plant expression studies.

Arabidopsis has been demonstrated and widely employed as a model flowering higher plant due to its impact size, short life cycle, high competency for transformation, and increasing understanding of its biochemical and genetic background. Arabidopsis transformation with the expression plasmids p35SGIN(6)-35SIGC (containing 35S::GUSn/Intn(6)::NOS and 35S::Intc/GUSc::NOS), p35SGIN-35SIGC (containing 35S::GUSn/Intn::NOS and 35S::Intc/GUSc::NOS), p35SGIN (containing 35S::GUSn/Intn::NOS), p35SGIN(6) (containing 35S::GUSn/Intn(6)::NOS), and p35SIGC(−)-Bar (containing 35S::Intc/GUSc::NOS) was carried out via Agrobacterium transformation.

Agrobacterium Transformation

To prepare competent agrobacterial cells, a colony of Agrobacterium strain C58C1 (pMP90) (Koncz et al., Mol. Gen. Genet., 204(3): 383-396 (1986)) was grown in 1 L YEP media, including 10 g Bacto peptone, 10 g yeast extract, and 5 g NaCl, until an OD₆₀₀ of 1.0 was reached. The culture was chilled on ice and the cells were collected by centrifugation. The competent cells were resuspended in ice cold 20 mM CaCl₂ solution and stored at −80° C. in 0.1 mL aliquots.

A freeze-thaw method was used to introduce expression plasmid constructs p35SGIN(6)-35SIGC, p35SGIN-35SIGC, p35SGIN, p35SGIN(6), and p35SIGC(−)-Bar into Agrobacteria. At first, 1 μg plasmid DNA from each construct was added to the frozen aliquoted agrobacterial cells. The mixture was thawed at 37° C. for 5 min, added to 1 mL YEP medium, and then gently shaken at 28° C. for 2 hrs. Cells were collected by centrifugation and grown on a YEP agar plate containing 25 mg/L gentamycin and 50 mg/L kanamycin at 28° C. for 2 to 3 days. Agrobacterial transformants were confirmed by minipreparation and restriction enzyme digestion of plasmid DNA by routine methods, except that lysozyme (Sigma, St. Louis, Mo.) was applied to the cell suspension before DNA preparation to enhance cell lysis.

Arabidopsis Transformation Arabidopsis thaliana was grown to bolting in 3″ square pots of Metro Mix soil (Scotts-Sierra, Maryville, Ohio) at a density of 5 plants per pot, under controlled temperature (22° C.) and illumination (16 hrs light/8 hrs dark). Plants were decapitated 4 days before transformation. Agrobacteria carrying expression plasmid constructs p35SGIN(6)-35SIGC, p35SGIN-35SIGC, p35SGIN, p35SGIN(6), and p35SIGC(−)-Bar were grown in LB medium (1% bacto-tryptone, 0.5% bacto-yeast extract, 1% NaCl, pH 7.0) containing 25 mg/L gentamycin and 50 mg/L kanamycin at 28° C., until the culture reached an OD₆₀₀ value of 1.2. Cells were collected by centrifugation and resuspended in infiltration medium (½×MS salt, 1×B5 vitamins, 5% sucrose, 0.5 g/L MES, pH 5.7, 0.044 μM benzylaminopurine) to an OD₆₀₀ of approx. 0.8.

Instead of a traditional vacuum infiltration method to transfect the Arabidopsis plants with the agrobacterium strains, transformation followed a simplified floral dip method (Clough, S. J. and A. F. Bent, Plant J. 16:735-43 (1998)). Briefly, the mid-log C58 Agrobacteria carrying the expression plasmids were resuspended in 5% sucrose with 0.05% Silwet L-77 (Lehle Seeds, Midland, Tex.) to an OD₆₀₀ of 0.8. Four- to five-week old flowering Arabidopsis plants were dipped into the Agrobacteria resuspension for 2 to 3 sec with agitation. The transfected plants were laid on their side, covered with a plastic dome, and placed in low light conditions for two days. They were then grown to maturation under standard conditions (22° C., 16 hrs light/8 hrs dark). Finally, seeds (including non-transformed and primary transformed ones (T1)) were collected from the plants. Usually, four to five pots of Arabidopsis were transinfected for each construct.

By applying the described methods, expression plasmids p35SGIN(6)-35SIGC, p35SGIN-35SIGC, p35SGIN, p35SGIN(6), and p35SIGC(−)-Bar (from Example 4) were introduced into Arabidopsis. Additionally, pGYV1-GUSM (containing 35S::GUS::NOS as a transgene) was also introduced into Arabidopsis as a positive control. For all transformants, primary transformed seeds (T1) were collected and named according to the following Table.

TABLE 4
Identification of Primary Transformed Seeds According to
Expession Plasmid Used for Transformation

| Expression | | Primary (T1) |
|---|---|---|
| Plasmid | Transgene | Transformed Seeds |
| p35SGIN(6)- | 35S::GUSn/Intn(6)::NOS and | A55 |
| 35SIGC | 35S::Intc/GUSc::NOS | |
| p35SGIN-35SIGC | 35S::GUSn/Intn::NOS and | A56 |
| | 35S::Intc/GUSc::NOS | |
| p35SGIN | 35S::GUSn/Intn::NOS | A57 |
| p35SGIN(6) | 35S::GUSn/Intn(6)::NOS | A58 |
| P35SIGC(−)-Bar | 35S::Intc/GUSc::NOS | A59 |
| PGYV1-GUSM | 35S::GUS::NOS | A54 |

### Example 6

### Identification and Examination of A55 (35S::GUSn/Intn(6)::NOS and 35S::Intc/GUSc::NOS) and A56 (35S::GUSn/Intn::NOS and 35S::Intc/GUSc::NOS) T1 Transgenic Plants

Example 6 describes the selection of successfully transformed plants from Example 5, the development of A55 and A56 seedlings from that transformation, and preliminary analysis of GUS expression in the leaves of those T1 seedlings. As expected, A56 plants containing GUSn/Intn and Intc/GUSc were able to undergo intein splicing to produce an active, mature GUS protein that could be visually detected. In contrast, A55 plants (containing GUSn/Intn(6) and Intc/GUSc) could not produce an active, mature GUS protein, since the intein-mediated splicing reaction was inhibited by the 6 amino acid deletion present in Intn(6).

Transformed seeds of A55 and A56 had been transinfected by the constructs of p35SGIN(6)-35SIGC and p35SGIN-35SIGC, respectively (Example 5). In addition to having transgene expression cassettes, they also carried the expression cassette NOS::NPT2::OCS, and thus could be identified by the Kan^{R} (kanomycin resistance) phenotype during their germination.

To select the transgenic plants, seeds from each T1 seed collection of A55 and A56 were sterilized in 80% ethanol with 0.01% Triton X-100 for 10 min, in 33% bleach with 0.01% Triton X-100 for 10 min, and finally rinsed in sterile water 5 times. Approximately 2,500 sterile seeds were placed on the top of a 120 mm selective plate consisting of 1×MS, 1% sucrose, 0.8% agar, 100 mg/L Timentin (SmithKline Beecham, Philadelphia, Pa.), 10 mg/L Benomyl (DuPont, Wilmington, Del.), and 50 mg/L kanamycin sulfate (Sigma, St. Louis, Mo.). They were subjected to 4° C. cold treatment for 2 days and then germinated under continuous illumination at 22° C. for 2 weeks. The transformed seeds germinated and grew into healthy T1 seedlings, while non-transformed seeds germinated but stopped growing and became bleached on the selective plates.

Each healthy seedling was transplanted to an individual 3″ pot containing MatroMix soil and grown under standard conditions (22° C., 16 hrs light/8 hrs dark) until maturation. T2 seeds were harvested from each plant to represent individual transformation events. In total, approximately 20,000 to 30,000 seeds for each T1 seed collection of A55 and A56 were screened on the selective plates. Thirty-six (36) A55 transgenic plants and 19 A56 transgenic plants were identified. The A54 T1 seed collection was also screened in the same way and 12 transgenic plants were identified for use as positive controls.

During the growth of these T1 transgenic plants, a portion of leaf (one-half) was collected from each plant for a preliminary GUS staining assay. At first, each piece of leaf was placed in an individual well of a 24-well titration plate. They were then embedded in 1.5 mL GUS staining solution (100 mM sodium phosphate buffer pH 7.0, 1 mM EDTA, 0.5 mM K₄ [Fe(CN)₆].3H₂O, 1 mM 5-bromo-4-chloro-3-indoyl β-D-glucuronide cyclohexiammonium salt, 0.5% Triton X-100) at 37° C. overnight. Finally, stained tissues were treated with 75% ethanol for a few days to remove the leafs natural color. As a result, tissue with positive GUS activity would show dark-blue staining while tissue with a negative GUS reaction appeared bleached.

All positive control (A54) transgenic plants, except one, showed positive GUS staining, but wild type non-transgenic plants had negative GUS reactions. Because these positive controls carried an expression cassette of 35S::GUS::NOS, the positive results demonstrated that the present transgenic plant system functioned as expected.

The A55 plants carried two transgene cassettes of 35S::IntC-GUSc::NOS and 35S::GUSN-Intn(6)::NOS. Because the first 6 codons of Ssp DnaE InteiN-n had been deleted in the second cassette (which were located within conserved motif A and included a cysteine critical in the protein splicing mechanism), the GUSN-Intn(6) fusion protein produced by this cassette did not have a functional intein-n and therefore could not undergo a protein trans-splicing reaction with the IntC-GUSc fusion protein produced by the first expression cassette to generate an intact GUS enzyme. Thus most of A55 transgenic plants (30 out of 36 plants) showed negative GUS staining. However, leaves from six A56 plants appeared slight pale-blue after GUS staining, indicating that protein splicing might be occurring with a very low efficiency.

The A56 plants carried two transgene expression cassettes of 35S::IntC-GUSc::NOS and 35S::GUSN-Intn::NOS. Their products included intact Ssp DnaE inteiN-c and inteiN-n, respectively, and could undergo protein trans-splicing and produce a complete GUS enzyme. As expected, leaves from each of the 19 A56 transgenic plants showed strong GUS staining. These results implied that the synthetic Ssp DnaE split intein containing plant optimized codons did permit introduction of a functional protein trans-splicing mechanism into transgenic plants.

### Example 7

### Examination of Protein Trans-Splicing in A55 (35S::GUSn/Intn(6)::NOS and 35S::Intc/GUSc::NOS) and A56 (35S::GUSn/Intn::NOS and 35S::Intc/GUSc::NOS) T2 transgenic plants

Example 7 is a detailed examination of the T2 seeds generated from representative A55 and A56 plants of Example 6. Visual assays for the functionality of the GUS protein throughout all tissues of adult transgenic plants were confirmed via analysis of genomic DNA, RNA transcriptions, and protein expression.

For a detailed molecular analysis of intein-mediated protein trans-splicing, T2 seeds were collected from two representative primary (T1) A55 transformants (plants A55-10 and A55-23) and two representative primary (T1) A56 transformants (plants A56-1 and A56-14). Additionally, T2 seeds were also collected from two primary (T1) A54 transformants (plants A54-1 and A54-9) and employed as positive controls, since these plants contained the fully functional 35S::GUS::NOS construct. All seeds were sterilized and germinated on kanamycin selective plates, as described previously. Two-week old seedlings were used in the below studies, unless mentioned specifically. In all cases, non-transformed seedlings were employed as negative controls.

Results of PCR, RNA blot assays, and protein immunoblot assays verified that the Ssp DnaE split intein, engineered to contain plant optimized codons, could mediate protein trans-splicing in plant cells. The splicing process not only ligated two extein fragments into a mature protein but also folded the protein into its active form.

GUS Staining Assay

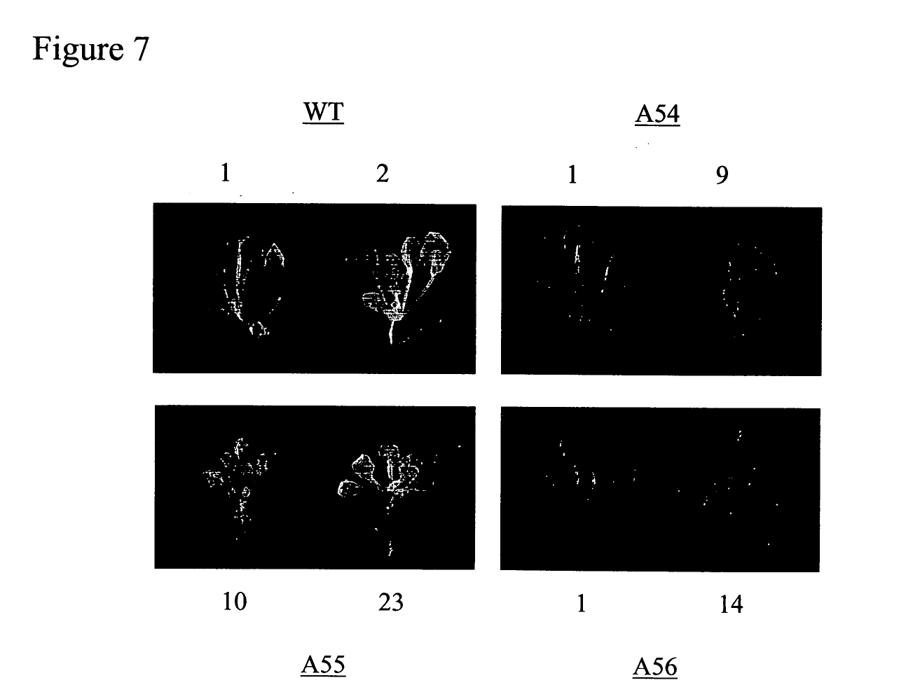

First, seedlings were subjected to GUS staining assays, as described in Example 6. FIG. 7 shows positive GUS staining in A56 seedlings (plants A56-1 and A56-14) and negative GUS staining for A55 seedlings (plants A55-10 and A55-23). The results confirmed the preliminary observations in Example 6 and indicated that the protein trans-splicing mechanism was both functional and heritable in transgenic plants.

Examination of Protein Trans-Splicing in Mature T2 Transgenic Arabidopsis Plants

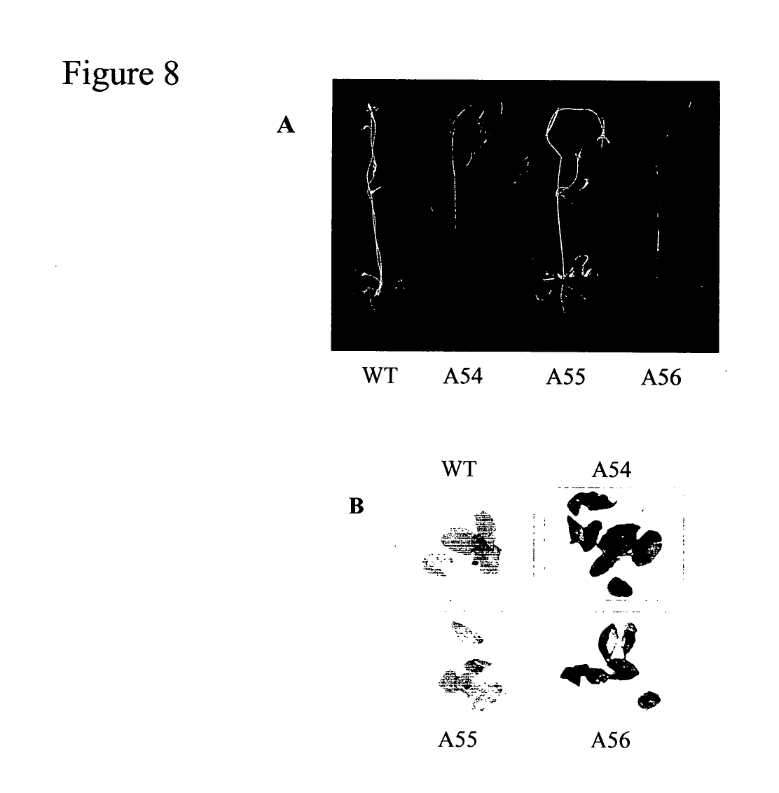

To determine if the conclusions drawn using seedlings extended to other tissue types in the plant, one A55 and one A56 seedling was grown to maturity in soil and then subjected to the GUS staining assay in 15 mL GUS staining solution. As expected, neither the A55 plant nor the non-transgenic plant displayed GUS activity in any part of the plant (FIG. 8A). Plant A56 displayed strong GUS activity throughout the plant, identical to the reaction in the positive control transgenic plant (A54).

Individual seeds showed results consistent with those of the whole plant, although some A56 seeds did not exhibit positive staining (possibly due to gene segregation) (FIG. 8B). These results demonstrated that the protein trans-splicing mechanism functions well in all types of tissues in plants, including leaf, stem, root, flower, and seed tissues. This mechanism could be utilized in a tissue-specific, developmental stage-specific, or environmental condition-specific manner, if driven by a suitable conditional promoter.

Confirmation of Intein-Mediated Protein Splicing Results by PCR Assay

Although all transgenic Arabidopsis were identified through kanamycin resistance screening, PCR assays were performed to directly examine integration of transgenes into the Arabidopsis genome. For these assays, approximately 30 ng (100 μL) DNA was prepared from A54, A55, and A56 seedlings by using 100 mg plant tissue and the DNeasy Plant Mini Kit (QIAGENE, Valencia, Calif.), following the manufacturers' instructions. One PCR reaction consisted of 25 μL of Plantnum PCR SuperMix, 1 μL (2.5 pmol) of each primer, and 1 μL (approximately 0.5 ng) DNA. The reactions were heated for 3 min at 98° C., followed by 35 cycles of 30 sec denaturation at 94° C., 30 sec annealing at 55° C., and 2 min amplification at 72° C. In all PCR assays, DNA from non-transformed plant was used as a negative control.

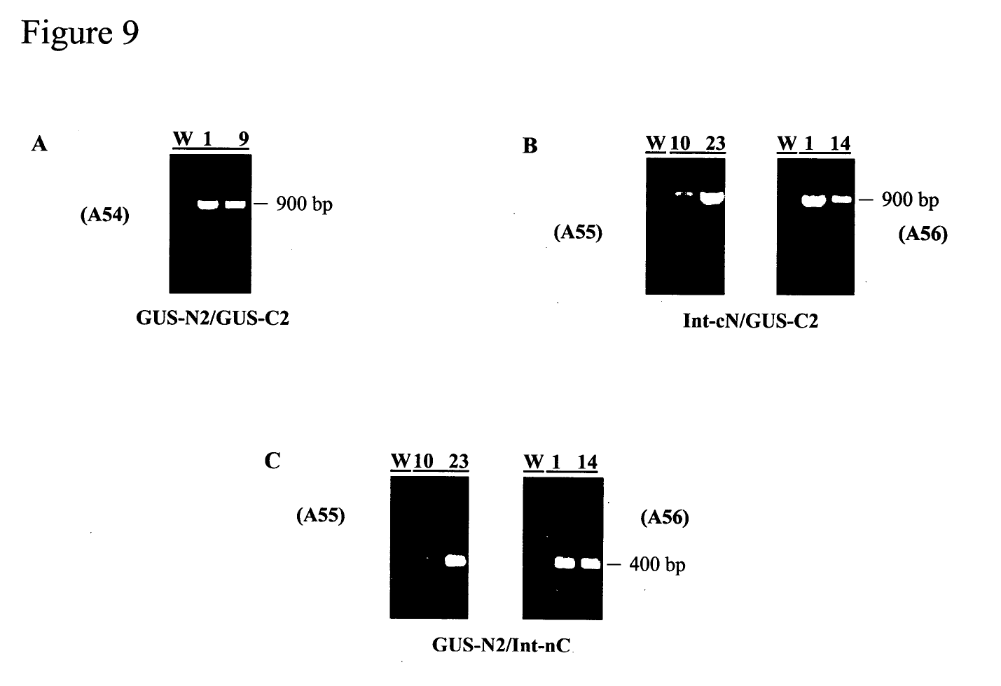

As shown in FIG. 9, primers GUS-N2 and GUS-C2 (SEQ ID NOs: 29 and 30) amplified a GUS fragment of approximately 900 bp from genomic DNA of A54 plants, thus confirming integration of expression cassette 35S::GUSM::NOS (FIG. 9A) in the positive controls. Primers Int-cN and GUS-C2 (SEQ ID NOs:20 and 30) amplified a IntC-GUSc fusion fragment of approximately 400 bp from genomic DNA of A55 and A56 plants, indicating integration of expression cassette 35S::IntC-GUSc::NOS (FIG. 9B, left and right panel). Primers GUS-N2 and Int-nC (SEQ ID NOs: 29 and 19) amplified a GUSN-Intn(6) fusion fragment of approximately 900 bp from A55 DNA and a GUSN-Intn fusion fragment of approximately 900 bp from A56 DNA, indicating integration of expression cassettes 35S::GUSN-Intn(6)::NOT and 35S::GUSN-Intn::NOT (FIG. 9C, left and right panel).

Confirmation of Intein-Mediated Protein Splicing bv RNA Blot Assays

To examine RNA expression of the transgenes, total RNA was purified from 700 mg of non-transformed seedlings (negative control), A54-1 and A54-9 T2 seedlings (positive control), and selected A55 and A56 T2 seedlings by using RNeasy Plant Mid Kits (QIAGENE, Valencia, Calif.). The protocol was provided by the manufacturer and RNA concentration was determined with spectral absorption at 260 nm. RNA expression was examined by RNA blot assay. At first, RNA samples (approximately 6 μg for each) were separated by RNA agarose gel electrophoresis in 1×MOPS gel running buffer (0.1M MOPS pH 7.0, 40 mM sodium acetate, 5 mM EDTA) at 100 volts for 3 hrs. The gel consisted of 1% agarose, 6% formaldehyde, and 1×MOPS gel running buffer. RNA samples were then blotted to Hybond-N+membrane (Amersham Pharmacia, Piscataway, N.J.) using a PosiBlot 30-30 Pressure Blotter (STRATAGENE, La Jolla, Calif.) under 75 mm Hg pressure for 1 hr, following the manufacturers' instructions.

A ³²p-α-dCTP labeled GUS probe was prepared by using a Random Primers DNA Labeling System (Life Technologies, Rockville, Md.), according to a protocol provided by the manufacturer. The GUS coding region was employed as a template. The synthetic probe was purifiedpn a Sephadex G-50 Nick-column (Amersham Pharmacia).

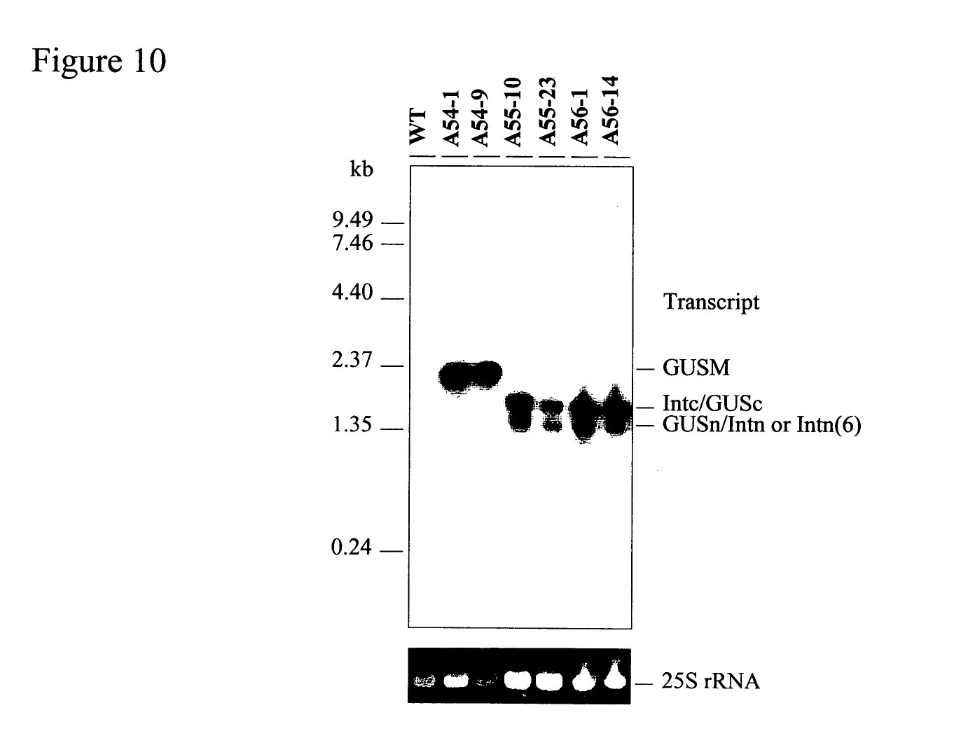

The RNA blots were incubated in 10 mL 65° C. Church-Gilbert hybridization solution (0.5 M sodium phosphate buffer pH 6.8, 7% SDS, 1% BSA, 1 mM EDTA) for 2 hrs. Approximately 5×10⁶ cpm of probe was added and incubation continued overnight. The membrane was washed for 3×10 min in 40 mM sodium phosphate buffer (pH 6.7) with 1% SDS. The results (shown in FIG. 10) were documented by exposing to BioMax X-ray film (Kodak, Rochester, N.Y.). A 1.4 kb transcript of 35S::GUSN-Intn(6)::NOS and a 1.8 kb transcript of 35S::IntC-GUSc::NOS were detected from A55 DNA, and a 1.4 kb transcript of 35S::GUSn/Intn::NOS and a 1.8 kb transcript of 35S::Intc/GUSc::NOS were detected from A56 DNA. A 2.2 kb transcript of 35S::GUSM::NOS was detected from positive controls (A54). No signal was detected in DNA prepared from non-transformed plants. Ethidium bromide stained 25S rRNA in the agarose gel is shown at the bottom of the figure, to indicate actual loading of each sample.

These results demonstrated the mRNA expression of transgenes in A55 and A56 transgenic Arabidopsis.

Confirmation of Intein-mediated Protein Splicing by Protein Immunoassays

To examine protein expression and assembly in transgenic Arabidopsis, protein extracts were made from the non-transformed seedlings (negative control), A54-1 and A54-9 T2 seedlings (positive control), and selected A55 and A56 T2 seedlings. Plant materials were ground into powder by motor in liquid nitrogen. 2×volume of protein extract buffer (50 mM Tris-HCl pH 7.5, 50 mM NaCl, 0.1 mM EDTA, 5 mM MgCl₂, 5% glycerol, 1% Sigma protein inhibitor cocktail) was added and ground further. The mixtures were centrifuged at 10 K×g for 10 min and the supernatants were saved as protein extracts. Protein concentration was determined by using BioRad Protein Assay reagent (Bio-Rad, Hercules, Calif.).

Protein products of the transgenes were determined by immunoblot assay. Since the fusion proteins and their splicing products possessed a 6×His tag either at their N- or their C-terminus, the immunoblot assay was carried out by using Penta-His antibody (QIAGEN, Valencia, Calif.) for detection of this 6×His tag on protein molecules. Briefly, 10 μL of the protein preparation (approximately 20 μg protein) was run on a 10% mini-SDS-PAGE gel at 100 volts for 1.5 hrs and transferred to 0.2 μm Protran nitrocellulose membrane (Schleicher & Schuell, Keene, N.H.) in ice at 100 volts for 1 hr. All equipment, pre-cast gels, and buffers were provided by Bio-Rad. The membrane was treated with the following solutions in order: (1) TTBS (0.02 M Tris-HCl pH7.5, 0.5 M NaCl, 0.1% TweeN-20) with 5% non-fat milk for 1 hr at room temperature; (2) TTBS with 0.1% Penta-His antibody overnight at 4° C.; (3) TTBS with 0.2% peroxidase-conjugated goat anti-mouse IgG (Jackson ImmunoResearch, West Grove, Pa.) for 2 hrs at room temperature; and (4) 0.1 M Tris-HCl (pH 8.0) for 5 min at room temperature. The membrane was washed three times by TTBS between treatments. His-tagged proteins were visualized in ECL solution (100 mM Tris-HCl pH8.0, 0.2 mM P-coumaric acid, 1.25 mM 3-aminophthalhydrazide, 0.01% hydrogen peroxide). The result was recorded on a Hyper ECL film (Amersham Pharmacia).

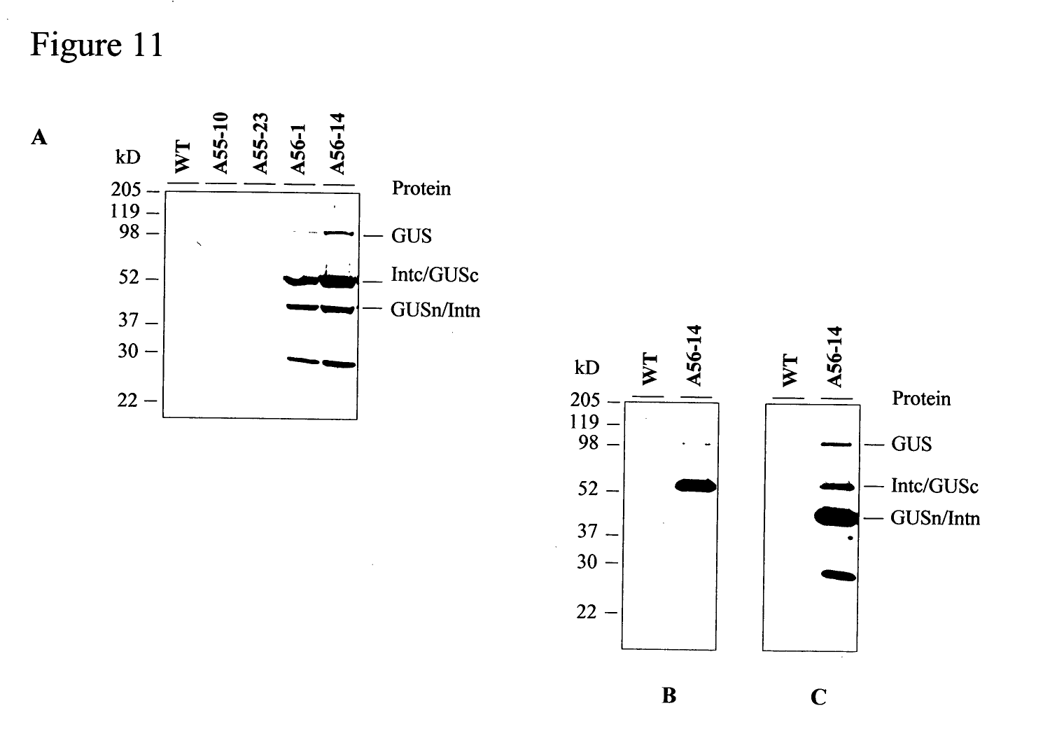

Immunoblot assay results are shown in FIG. 11A. This result indicated that GUSn/Intn (calculated molecular mass 37.4 kD) and Intc/GUSc (calculated molecular mass 50.7 kD) fusion proteins were synthesized from 35SP::GUSn/Intn::NOS and 35S::Intc/GUSc::NOS expression cassettes in A56 transgenic plants, respectively. Due to the intein-mediated protein trans-splicing mechanism, GUS fragments from these fusion proteins had been ligated into mature GUS proteins with a calculated molecular mass of 68.2 kD (validated by positive GUS staining). During the splicing, intein sequences were excised from fusion proteins, but they were not detectable in the experiments herein since they did not possess a His tag. In contrast, mature GUS protein could not be synthesized from GUSn/Intn(6) and Intc/GUSc fusion proteins produced in A55 transgenic plants because the 6-amino acid deletion mutation in Int-N had abolished the protein trans-splicing process. In fact, GUSn/Intn(6) and Intc/GUSc fusion proteins could not be detected from A55 plants although their mRNA expression profiles were similar to those in A56 plants, implicating that all these fusion proteins may be very unstable in plant cells unless strong interactions exist between intein-N and intein-C fragments. Results for A54 plants are not included, since the GUS protein produced therein did not have an attached 6×His tag permitting its detection.

In FIG. 11A, GUS, Intc/GUSc, and GUSn/Intn in A56 were larger than expected. An unknown protein smaller than 30 kD was also detected from A56 extract using the penta-His antibody. To clarify assignment of these proteins, His-tagged proteins were purified from the A56-14 protein extract. For purification, 600 μL of the protein extract was loaded on an equilibrated Ni-NTA spin column (QIAGEN). The 6×His tagged proteins were bound to the column, washed, and eluted with 200 μL of a high concentration imidazole solution (QIAGEN). The purified fraction was concentrated 5×fold with a Microcon spin tube. Protein extract from a nontransformed plant was also purified and concentrated to serve as a negative control. Twenty microliters of the concentrated fractions were used for immunoblot assay following the protocol described previously, however, anti-His(C-term)-HRP antibody (Invitrogen) was used to specifically detect C-terminal His-tags. After the assay, the blot was stripped in a solution containing 0.5 M NaCl and 0.2 M glycine (pH 2.8) and then subjected to a second immunoblot assay by using penta-His antibody as primary antibody. The result of the first assay is shown in FIG. 11B, while the second assay result is shown in FIG. 11C.

A comparison between the first and second immunoblots indicated that only the top two heavier proteins possessed C-terminal 6×His tags, thus confirming these proteins as mature GUS protein and the Intc/GUSc fusion protein. The bottom two proteins only have N-terminal 6×His tags. Based on size, the larger protein was confirmed as the GUSn/Intn fusion protein, while the smaller, unexpected protein fragment, is probably generated from degradation of GUSn/Intn.

### Example 8

### Transformation, Selection, and Genetic Typing of A57 (35S::GUSn/Intn::NOS), A58 (35S::GUSn/Intn(6)::NOS), and A59 (35S::Intc/GUSc::NOS) Transgenic Plants

Example 8 describes the transformation and selection of A57, A58, and A59 plants, based on antibiotic resistance. Detailed molecular analysis of each transgenic line was further conducted via PCR, RNA transcription, and protein expression.

Previous examples demonstrated protein trans-splicing between the proteins synthesized from two different transgenes of one locus in plant cells. However, if splicing would occur between transgene products produced from different loci or chromosomes, significant advantages for the application of trans-splicing in transgenic plant systems would be realized. Specifically, different transgenes could be brought into the same plants through plant breeding. To demonstrate this hypothesis, A57, A58, and A59 transgenic plants were identified and were crossed as described below.

A57, A58, and A59 seeds were transinfected by the constructs of p35SGIN, p35SGIN(6), and p35SIGC(−)-Bar, respectively. In addition to having the transgene expression cassettes, A57 and A58 carried the expression cassette of NOS::NPTII::OCS, and thus could be identified by the Kan^{R} (kanomycin resistance) phenotype during their germination. Screening of approximately 10,000 T1 seeds on kanamycin-containing selective plates resulted in 8 A57 and 13 A58 primary (T1) transgenic seedlings. A59 carried the expression cassette of NOS::Bar::NOS and could be identified by the BarR (glufosinate ammonium resistance) phenotype. Twenty-two Bar resistance A59 seedlings were identified from approximately 20,000 T1 seeds on selective plates, where 50 mg/L kanamycin sulfate was replaced by 20 mg/L glufosinate ammonium. All transgenic seedlings were grown up in soil. One-half leaf of each primary transgenic plant was subjected to preliminary GUS assay and all were negative (data not shown) compared to A54 positive controls. T2 seeds were collected from each individual plant, separately.

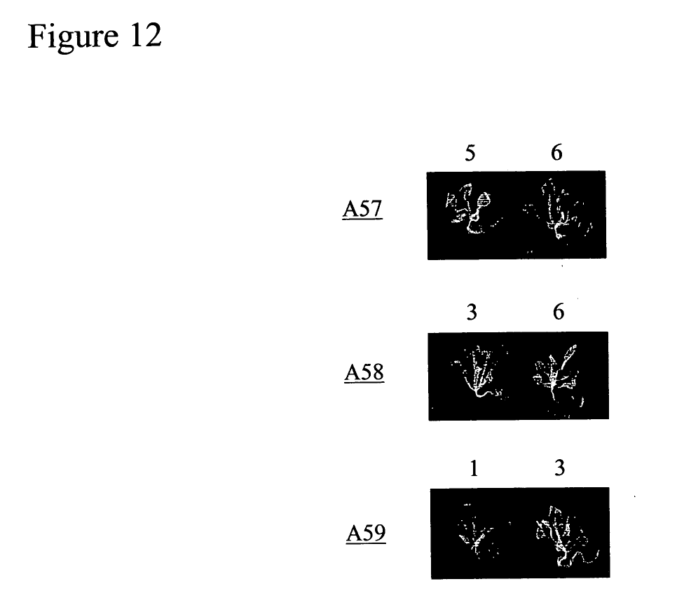

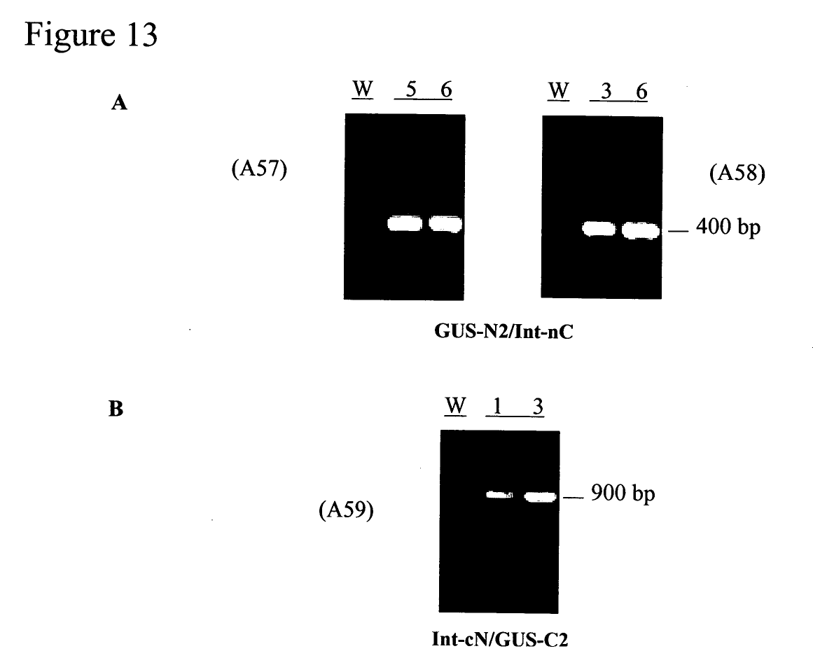

To further examine transgene expression in T2 transgenic plants, T2 seeds of A57-5, A57-6, A58-3, and A58-6 were germinated on Kan-selective plates, while those of A59-1 and A59-3 were germinated on Bar-selective plates. Two-week old healthy seedlings were used for GUS staining. All showed negative GUS staining (FIG. 12), further confirming that only half of the GUS protein was not sufficient to produce an active protein.

DNA, RNA, and protein was prepared from these seedlings and used for PCR, RNA blot assays, and protein assays, as described in Example 7. Comparable samples were prepared from non-transgenic plants for use as negative controls.

PCR primers GUS-N2 and Int-nC (SEQ ID NOs: 29 and 19) amplified a GUSn/Intn fusion fragment of approximately 400 bp from A57 DNA and a GUSn/Intn(6) fusion fragment of approximately 400 bp from A58 DNA, indicated integration of expression cassettes of 35S::GUSn/Intn::NOT and 35S::GUSn/Intn(6)::NOT (FIG. 13A, left and right panel). Primers Int-cN and GUS-C2 (SEQ ID Nos: 20 and 30) amplified a IntC-GUSc fusion fragment of approximately 900 bp from A59 DNA, indicated integration of expression cassette of 35S::Intc/GUSc::NOT (FIG. 13B).

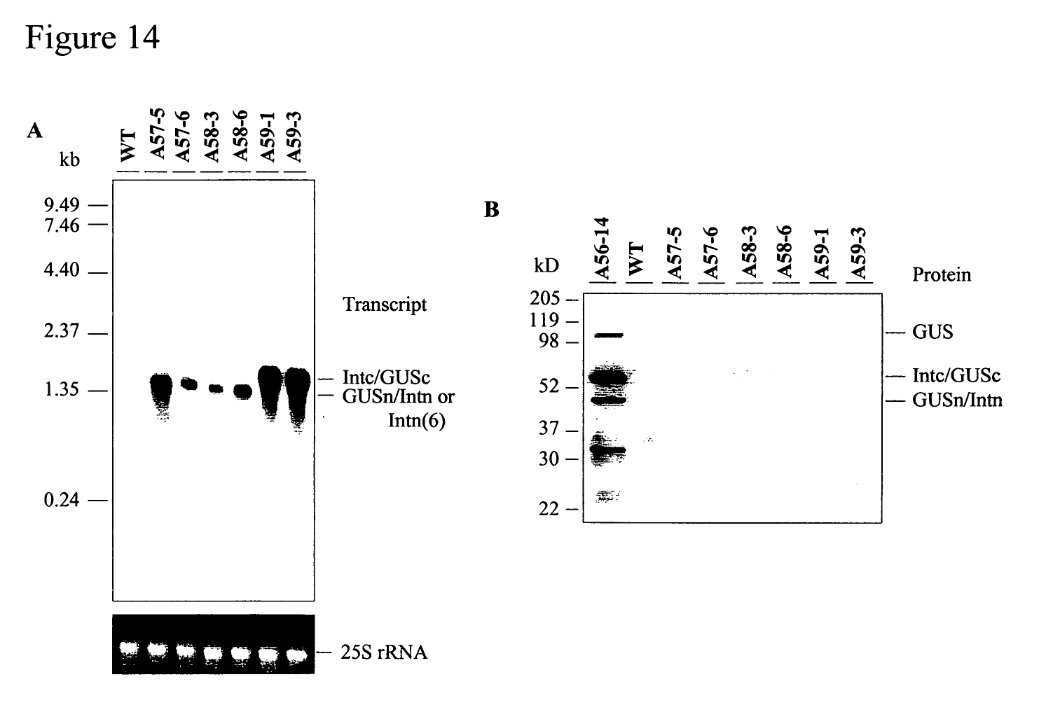

Results from the RNA blot assay are shown in FIG. 14A, demonstrating the expected mRNA expression of transgenes in each group of transgenic seedlings. Again, ethidium bromide stained 25S rRNA in the agarose gel is shown at the bottom of the figure, to indicate actual loading of each sample.

Protein samples were subjected to immunoblot assay. Penta-His antibody and peroxidase-conjugate goat anti-mouse IgG were employed as the primary and secondary antibody. FIG. 14B shows that there was no detectable accumulation of the transgene products in the A57, A58, or A59 plants. This result indicated that, without interaction between Int-N and Int-C, split GUS proteins were unstable in plants cells.

### Example 9

### Genetic Crossing between A57 (35S::GUSn/Intn::NOS), A58 (35S::GUSn/Intn(6)::NOS). and A59 (35S::Intc/GUSc::NOS) Transgenic Plants and Examination of Hybrid Progenies

Example 9 demonstrates that the progeny of a genetic cross between an N-plant host (containing an N-nucleotide sequence of P-ExtN-IntN) and a C-plant host (containing a C-nucleotide sequence of P-IntC-ExtC) are able to undergo intein-mediated protein splicing to create an active GUS protein.

To perform genetic crossing between the transgenic plants, homozygous A57, A58, and A59 seedlings were selected. Approximately 200 T2 seeds from A57 and A58 seed collections were germinated on Kan-selective plates. Those from the A59 seed collection were germinated on a Bar-selective plate. After two weeks under conditions of 22° C. and continuous illumination, the numbers of green healthy and pale dying seedlings were counted, respectively, and subjected to a Chi Squared statistical test. As a result, A57-6, A58-6, and A59-1 T2 seedlings were identified as having a single transgene insertion, showing a typical 3:1 segregation ratio. Thus eight T2 seedlings of A57-6, A58-6, and A59-1 were transplanted into individual pots and grown in soil under standard conditions. T3 seeds were collected from individual plants and geminated on appropriate selective plates. All seedlings of A57-6-3, A58-6-2, and A59-1-1 were resistant to their selective pressures and identified as homozygous plants. These plants were grown in soil and subjected to genetic crossing.

A59, which carried expression cassette 35S::Intc/GUSc::NOS, was selected as a pollen donor (male). Fully open flowers (with petals at a 90° C. angle) were chosen for pollen collection from A59 homozygous plants. A large amount of pollen was examined microscopically.

A57 and A58, respectively carrying 35S::GUSn/Intn::NOS and 35S::GUSn/Intn(6)::NOS, were selected as pollen recipients (female). The stigma of the pollen recipient was prepared by choosing several large unopened buds on a bolt with a stiff stalk on a young, hardy A57 or A58 homozygous plant. All siliques, open flowers, young buds, and meristem were removed. Then all sepals, petals, and anthers were removed from the chosen mature buds, to allow exposure of the stigma.

For genetic crossing, A59 pollen was dusted onto previously prepared A57 and A58 stigmas, respectively. Pollinated stigmas were wrapped up with small squares of plastic wrap for a few days to retain moisture and prevent further pollination. Plants were then grown under standard conditions, and Fl hybrid seeds were collected as A57×A59 and A58×A59, separately. Hybrids were confirmed by germinating A57×A59 and A58×A59 seeds on Kan-Bar-selective plates (50 μg/mL kanamycin sulfate and 20 μg/mL glufosinate ammonium). Kan-Bar-resistance seedlings (F1) were further grown in soil until maturation.

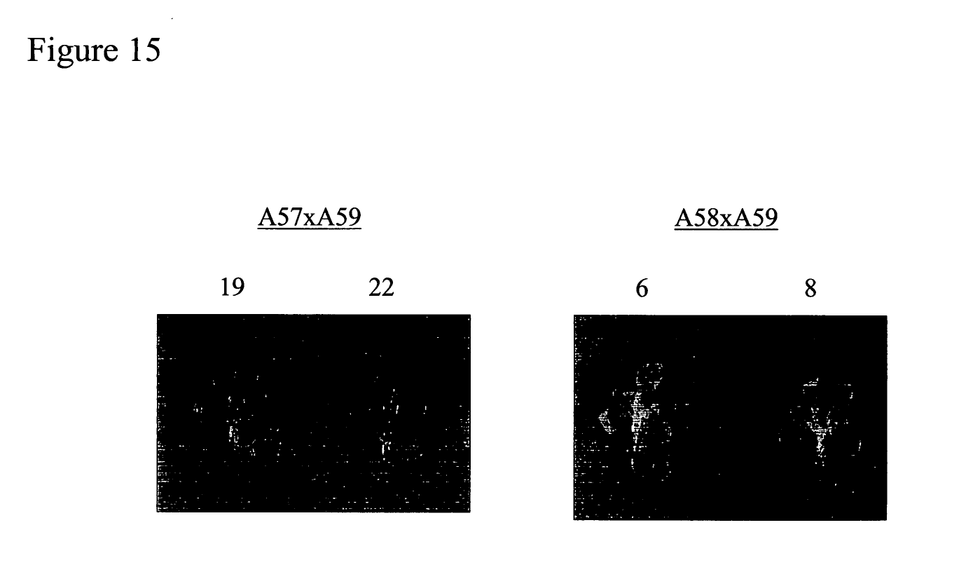

For examination of the hybrids, F2 seeds of A57×A59-19, A57×A59-22, A58×A59-6, and A58×A59-8 were germinated on Kan-Bar-selective plates. Because of transgene segregation, only a portion of the seeds were hybrid progenies which could be germinated on Kan-Bar-selective plates. Two-week old F2 seedlings were used for GUS assays (FIG. 15). The A57×A59 hybrid plant (containing p35SGIN and p35SIGC) demonstrated that intein cassettes obtained through genetic crossing enabled a fully functional intein-mediated trans-protein splicing reaction. In contrast, the mutated intein in A58×A59 plants (containing p35SGIN(6) and p35SIGC) abolished the normal function of the intein.

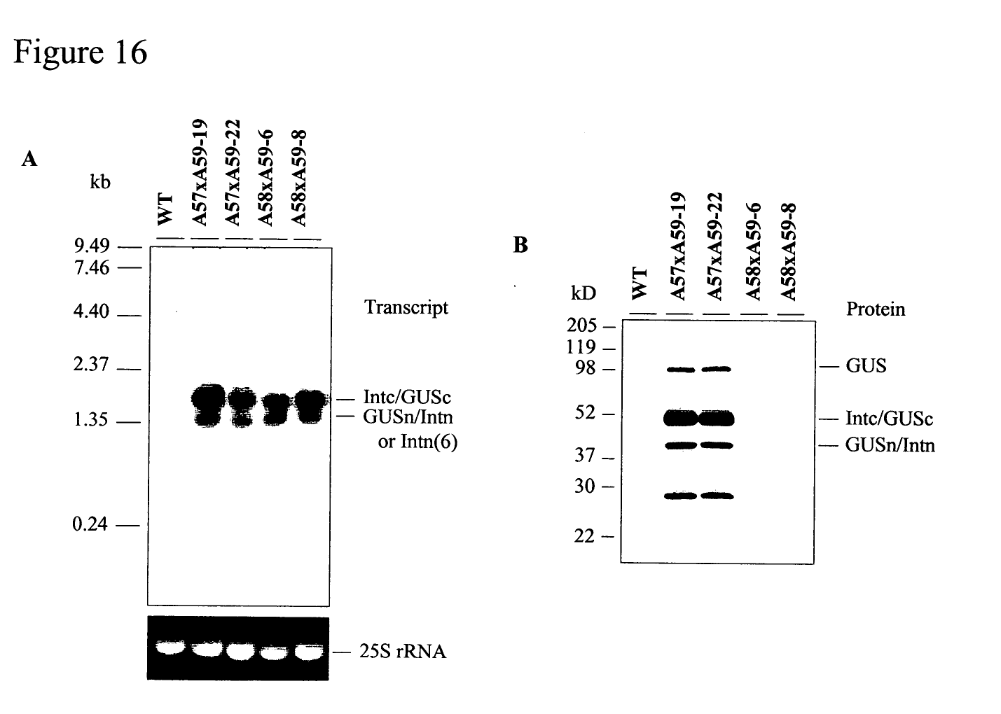

RNA and protein extracts were prepared from the seedling and used in a RNA blot assay and an immunoblot assay, as described in previous Examples. FIG. 16A shows results of the RNA blot assay, demonstrating that both intein cassettes in A57×A59 or A58×A59 plants were expressed as separate transcripts. Penta-His antibody was used in the immunoblot assay to detect protein splicing. The results in FIG. 16B confirmed intein-mediated GUS protein splicing in A57×A59 plants and malfunction due to the mutated intein sequence in A58×A59, consistent with GUS assay data.

Based on these results, it is concluded that the intein-mediated trans-protein splicing mechanism can be established in plant cells by placing two intein cassettes in the same locus, in separate loci, or in separate chromosomes. Genetic crossing, which brings the two cassettes into the same cell, can turn the protein splicing mechanism “on” and thus control the function of the intein.

### Example 10

### In Vitro Assembly of Mature and Active Protein by Intein-Mediated Trans Protein Splicing

Example 10 describes purification of the protein precursors GUSn/Intn and Intc/GUSc from A57 (35S::GUSn/Intn::NOS) and A59 (35S::Intc/GUSc::NOS) plants, followed by an in vitro intein splicing reaction to produce active, mature GUS protein.

Synthetic and natural split inteins can catalyze the protein splicing reaction in vitro. Synthetic inteins usually require denaturing conditions (i.e., high concentrations of urea) to complete the reaction, while natural inteins only need mild conditions. In vitro splicing could broaden the applications of plant-based intein technology in such areas as toxic protein synthesis and hybrid polymer assembly between synthetic and protein materials.

To demonstrate in vitro protein splicing, GUSn/Intn and Intc/GUSc fusion protein precursors can be produced in A57 and A59, respectively (Example 9). Due to very low abundance of the fusion proteins in these plants, they can be purified from large amounts of plant material using Ni-NTA affinity chromatographic methods, taking advantage of the His-tags on both fusion proteins. Proteins can be collected from Ni-NTA by eluting with high concentrations of imidazole (see Example 7).

For splicing, the buffer must possess an optimized pH value, ion strength, and dithiothreitol concentration. It can be achieved by dialyzing the purified protein precursors against an appropriate buffer. Equal concentrations of purified GUSn/Intn and Intc/GUSc protein are then mixed together and the reaction is performed at room temperature overnight.

GUS protein assembly is monitored by immunoblot assay, as described in the previous examples. GUS activity is examined by fluorescence assay using MUG (4-methyl umbelliferyl glucuronide) as a fluorogenic substrate. It is predicted that both GUS protein assembly and GUS enzymatic activity will be observed in the reactions.

### Example 11

### Transformation and Examination of Tobacco, Soy, Pea, Maize, and Barley

This example describes the transformation of binary vector-based expression plasmids from Example 4 into tobacco, soy, pea, maize, and barley.

Although the intein-mediated protein trans-splicing mechanism has been demonstrated in Arabidopsis, its utility in other plants (especially with agriculture-important crops) had not been tested, prior to the work described below. To demonstrate the compatibility intein-mediated splicing mechanism, leaf tissues were collected from 2-week old tobacco, soy, pea, maize, and barley plants. Expression plasmids p35SGIN(6)-35SIGC and p35SGIN-35SIGC (Example 4) were each introduced into each of the collected leaf tissue by biolistic bombardment.

The method employed for biolistic transformation was that of Maliga, P., et al. (In Method in Plant Molecular Biology, A Laboratory Course Manual. CSHL: Cold Spring Harbor, N.Y. (1995); pp 37-54). Briefly, 8 μg DNA of each expression plasmid was coated with 3 mg Biolistic 1.0 Micron Gold (Bio-Rad, Hercules, Calif.) in a solution of 1 M CaCl₂ and 15 mM spermidine, by vortexing for 3 min. Approximately 1.5 μg coated plasmid DNA was loaded on a PDS-1000/He Biolistic Particle Delivery System (Bio-Rad) and shot into 2-week old leaf tissue which was placed on a plate containing 1×MS salt, 0.8% agar, and 2% sucrose (protocol provided by the manufacturer). Delivery pressure was set at 1100 psi and distance at 10 cm. The treated tissue was recovered for 2 days on the same plate, under continuous light.

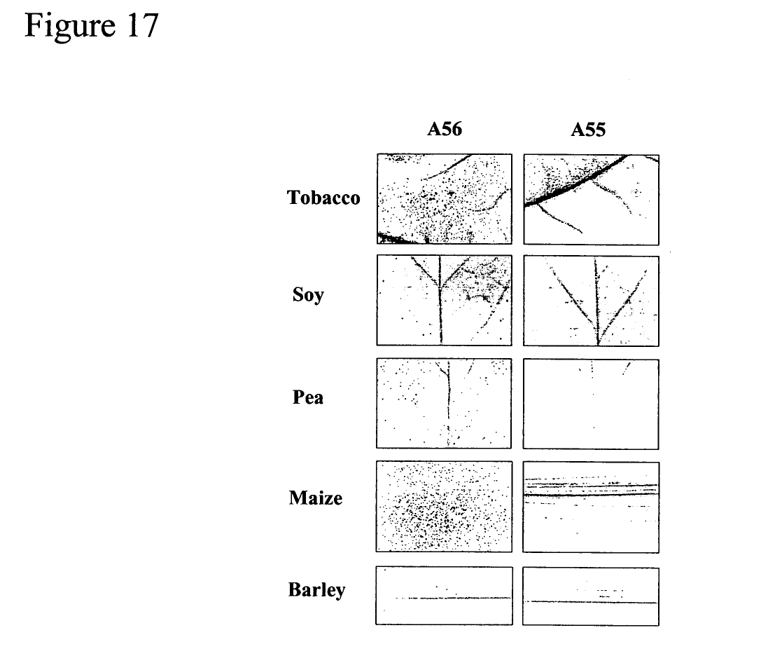

After two days recovery, re-assembly of β-glucuronidase in the transformed cells was examined by a GUS staining assay (as described in Example 6). All staining reactions were performed in triplicate, with one representative result for transformation with plasmid p35SGIN(6)-35SIGC (A55 plants) and p35SGIN-35SIGC (A56 plants) shown in FIG. 17 for each plant species.

The intein-mediated protein trans-splicing mechanism was reconstituted and intein-GUS fusion proteins were synthesized in transformed cells of all A56 leaf tissue. Thus, positive GUS staining was observed. In contrast, transformed cells in A55 leaf tissues showed negative GUS staining, since the 6-amino acid deletion mutation in Intn(6) had abolished the protein trans-splicing process. These results are consistent with those observed in Arabidopsis.

Finally, these results imply that Ssp DnaE split inteins function in both monocot- and dicot-plants for catalysis of protein trans-splicing. This is suggested since tobacco, soy, and pea are dicot-plants, while maize and barley are monocot-plants.

### Example 12

### Construction of Cre Recombinase-Intein Elements

The present Example describes the construction of an N- and C-nucleotide sequence using Cre recombinase enzyme as the extein. The bacterial Cre gene was artificially “split” into 2 portions, representing ExtN and ExtC. Then, split intein-Cre fusions were made to produce the two distinct intein cassettes (P-CreN-IntN on plasmid pGV947 and P-IntC-CreC on plasmid pGV951), each controlled by a promoter (P) suitable to drive the expression of CreN-IntN and IntC-CreC. The starting plasmid for making both IntN-CreN and IntC-CreC genes was pNY102, which contains a plant gene encoding a modified bacterial Cre.

Construction of Plasmid pNY102

pNY102 was made by converting the XbaI site in pSK (Stratagene) into an Asp718 site and cloning an Asp718 fragment containing the chimeric transgene, 35S promoter:Cre ORF:3′ octopine synthase (OCS) region, which encodes a functional Cre recombinase.

The 1411 bp region between Asp718 and the initiation codon of Cre ORF contains (5′ to 3′):

18 bp polylinker sequence, 5′-GGTACCCGATCCAATTCC-3′ (SEQ ID NO: 43);

1334 bp of 35S promoter that is similar to nucleotides 3114 to 4453 in cloning vector pKANNIBAL [Genbank accession No. AJ311873; Wesley, V. S., et al. Plant J. 27 (6): 581-590 (2001)]; and

60 bp 5′ UTR of Petunia gene for chlorophyll a/b binding protein cab 22L [nucleotides 171-230 Genbank accession no. X02359. Dunsmuir, P. Nucleic Acids Res. 13(7): 2503-2518 (1985)].

The Cre ORF is for bacteriophage P1 Cre gene for recombinase protein (Genbank accession No. X03453 and in Sternberg, N. et al. J. Mol. Biol. 187(2): 197-212 (1986)) except for a single base pair change (T to G) that was made at the fourth base of the ORF in order to introduce a Nco I site at the ATG, i.e., CCATGG, where the ATG is the initiation codon for Cre ORF, and resulting in a single amino acid substitution [Ser to Ala] at the second amino acid of the encoded Cre protein.

The 3′ OCS region [complement of nucleotides 12541-11835 in Genbank accession No. X00493 J05108 X00282; Barker, R. F., et al. Plant Mol Biol. 2: 335-350 (1983)] is flanked by Sal I/Xba I sites at the 5′ end and Asp 718 site at its 3′end.

Construction of Plasmid pGV947 Containing the Chimeric Gene Encoding the CreN-IntN Protein Fusion

A 483 bp PCR product encoding the N-terminal 155 amino acid sequence (M to C) of the modified bacterial Cre protein described above was made using upper primer SEQ ID NO:44 and lower primer SEQ ID NO:45 on pNY102. Upper primer SEQ ID NO:44 contains a Nco I site with an ATG codon that serves as the translation initiation methionine of the Cre ORF. The 5′ end of lower primer SEQ ID NO:45 contains a 13 bp sequence that is complementary to the 5′ end of the DNA sequence encoding IntN ORF.

A 394 bp PCR product encoding the 123 amino acid sequence (C to K) of IntN protein was made by using upper primer SEQ ID NO:46 and lower primer SEQ ID NO:47 on plasmid PInt-n containing the IntN gene (from Example 1). The 5′ end of SEQ ID NO:46 contains 14 bp of the sequence that is complementary to the 3′ end of the CreN region described above and that overlaps SEQ ID NO:45. The 3′ end of primer SEQ ID NO:47 contains a Sal I site.

A 849 bp PCR product encoding the complete 278 amino acid sequence of the CreN-IntN fusion protein was made by using upper primer SEQ ID NO:44 and lower primer SEQ ID NO:47 on a mixture of the 483 bp and 394 bp PCR products. The 3′ end of the 483 bp fragment and the 5′ end of the 394 bp fragment had a 27 bp sequence overlap. The 849 bp PCR product was cloned into pGEMT Easy vector (Stratagene) to yield plasmid pGV942 in which the Sal I site from the PCR product is adjacent to the Spe I site in the vector and its sequence was confirmed.

The 839 bp Nco I-Spe I fragment containing the CreN-IntN ORF was isolated from pGV942 and cloned into pNY102 to replace the Nco I-Xba I fragment containing full length Cre ORF to yield pGV947. Thus, pGV947 contains the chimeric 35S promoter: CreN-IntN ORF: 3′ ocs transgene in a 3034 bp Asp718 fragment (SEQ ID NO:48) that is comprised of (5′ to 3′):

18 bp (nucleotides 1-18) polylinker sequence, 5′-GGTACCCGATCCMTTCC-3′ (SEQ ID NO:43);

1334 bp (nucleotides 19-1352) of 35S promoter that is similar to nucleotides 3114 to 4453 in cloning vector PKANNIBAL [Genbank accession No. AJ311873; Wesley, V. S., et al. Plant J. 27(6): 581-590 (2001)];

60 bp (nucleotides 1353-1412) 5′ UTR of Petunia gene for chlorophyll a/b binding protein cab 22L [nucleotides 171-230 Genbank accession no. X02359; Dunsmuir, P. Nucleic Acids Res. 13(7): 2503-2518 (1985)];

837 bp (nucleotides 1413-2249) CreN-IntN ORF;

17 bp (nucleotides 2250-2266) sequence, 5′-GTCGACATMTCACTAG-3′ (SEQ ID NO:49);

708 bp (nucleotides 2267-2974) 3′ OCS region [complement of nucleotides 12541-11835 in Genbank accession no. X00493 J05108 X00282; Barker, R. F., et al. Plant Mol. Biol. 2: 335-350 (1983)]; and

60 bp (nucleotides 2975-3034) polylinker sequence, 5′-

| | |
|---|---|
| CAGGACCTGCAGGCATGCAAGCTTATCGATACCGTCGACCTC | (SEQ ID |
| GAGGGGGGGCCCGGTACC-3′. | NO: 50) |

Construction of Plasmid pGV951Containing the Chimeric Gene Encoding the IntC-CreC Protein Fusion

A 128 bp PCR product encoding the 111 amino acid sequence of IntC ORF was made by using upper primer SEQ ID NO:51 and lower primer SEQ ID NO:52 on plasmid pINT-C containing the IntC gene (from Example 1). Upper primer SEQ ID NO:51 contains a Nco I site with an ATG codon that serves as the translation initiation methionine of the IntC ORF. The 5′ end of the lower primer SEQ ID NO:52 contains a 13 bp sequence that is complementary to the 5′ end of the DNA sequence encoding the C-terminal portion of the Cre protein (see below).

A 588 bp PCR product (CreC) encoding the 564 amino acid sequence (Q to D) of the C-terminal portion of the bacterial Cre protein was made by using primers SEQ ID NO:53 and SEQ ID NO:54 on plasmid pNY102. The 5′ end of SEQ ID NO:53 contains 13 bp of the sequence that is complementary to the 3′ end of the IntC ORF and overlaps primer SEQ ID NO:52. The 3′ end of SEQ ID NO:54 contains a Sal I site outside (i.e., 3′ to) the CreC ORF.

A 688 bp PCR product containing the 225 amino acid sequence of the IntC-CreC fusion protein was made by using upper primer SEQ ID NO:47 and lower primer SEQ ID NO:50 on a mixture of the 128 bp and 588 bp PCR products. The 3′ end of the 128 bp and the 5′ end of the 588 bp fragments had a 26 bp sequence overlap. The 688 bp PCR product was cloned into pGEMT Easy vector (Stratagene) to yield plasmid pGV943 in which the Sal site in the PCR product was adjacent to the Spe I site in the vector and its sequence was confirmed.

The 680 bp Nco I-Spe I fragment containing the CreN-IntN ORF was isolated from pGV943 and cloned into pNY102 to replace the Nco I-Xba I fragment containing full length Cre ORF to yield pGV951. pGV951 contains the chimeric 35S promoter: IntC-CreC ORF: 3′ ocs transgene in a 2868 bp Asp718 fragment described by the 2873 bp sequence in SEQ ID No. 55 that is comprised of (5′ to 3′):

18 bp (nucleotides 1-18) polylinker sequence, 5′-GGTACCCGATCCAATTCC-3′ (SEQ ID NO:43);

1334 bp (nucleotides 19-1352) of 35S promoter that is similar to nucleotides 3114 to 4453 in cloning vector pKANNIBAL [Genbank accession No. AJ311873; Wesley, V. S., et al. Plant J. 27(6), 581-590 (2001)];

60 bp (nucleotides 1353-1412) 5′ UTR of Petunia gene for chlorophyll a/b binding protein cab 22L [nucleotides 171-230 Genbank accession no. X02359; Dunsmuir, P. Nucleic Acids Res. 13(7): 2503-2518 (1985)];

678 bp (nucleotides 1413-2090) IntC-CreC ORF;

15 bp (nucleotides 2091-2105) sequence, 5′-GTCGACTATCACTAG-3′ (SEQ ID NO:56);

708 bp (nucleotides 2106-2813) 3′ OCS region [complement of nucleotides 12541-11835 in Genbank accession no. X00493 J05108 X00282; Barker, R. F., et al. Plant Mol. Biol. 2: 335-350 (1983)]; and

60 bp (nucleotides 2814-2873) polylinker sequence, 5′-

| | |
|---|---|
| CAGGACCTGCAGGCATGCAAGCTTATCGATACCGTCGACCTC | (SEQ ID |
| GAGGGGGGGCCCGGTACC-3′. | NO: 50) |

### Example 13

### Making Reporter Plasmid pGV801 as a Trait Expression Construct

Example 12 describes the construction of a trait expression construct in plasmid pGV801, containing the reporter gene encoding β-glucuronidase (GUS). This “trait expression construct” is a genetic construct containing the generic structure: P-LoxP-STP-LoxP-TG, whereby P is a promoter driving the expression of the trait gene (TG), Lox is a site specific recombinase site recognized by the Cre site specific recombinase enzyme, STP is any blocking fragment of DNA, and TG is the trait gene. Thus, activation of the trait gene is not able to occur until removal of the blocking fragment, which can occur since the trait expression construct is a substrate for site-specific recombination. Once the blocking fragment is removed by site specific recombination, transcriptional and/or translational expression of TG will result.

A reporter plasmid construct pGV801 was made containing a 35S promoter: LoxP:nos:npt II:3′nos:LoxP:GUS ORF:3′ nos cassette. In it, the plant kanamycin resistance gene (nos:nptll:3′nos is a chimeric noplaine synthase (nos) promoter: neomycin phosphotransferase:3′ nos transgene) flanked by loxP sites is inserted as a blocking fragment between a 35S promoter and the β-glucuronidase (GUS) coding region. The blocking fragment blocks the translation of GUS by interrupting the GUS coding sequence. However, upon Cre-lox excision, there is a single copy of the loxP site left behind as a translational fusion with the GUS ORF thereby allowing glucuronidase expression.

The reporter plasmid construct, named pGV801, harbors the 5449 bp Sal I-Hind III fragment ((SEQ ID NO:57), which contains the blocked reporter construct, 35S promoter: LoxP:nos:npt II:3′nos:LoxP:GUS ORF:3′ nos, and is comprised of (5′ to 3′):

24 bp (nucleotides 1-24) polylinker sequence, 5′-GTCGACTCTAGAGGATCCM TTCC-3′ (SEQ ID NO:58);

1334 bp (nucleotides 25-1358) of 35S promoter (similar to nucleotides 3120 to 4453 in cloning vector pKANNIBAL [Genbank accession No. AJ311873), although with a unique Bgl II site at position 405-410;

60 bp (nucleotides 1359-1418) 5′ UTR of Petunia gene for chlorophyll a/b binding protein (corresponding to nucleotides 171-230, Genbank accession no. X02359);

3 bp (nucleotides 1419-1421) of initiation codon ATG;

34 bp (nucleotides 1422-1455) Lox P sequence (5′-ATMCTTCGTATAGCATAC ATTATACGAAGTTAT-3′) (SEQ ID NO:59);

5 bp (nucleotides 1456-1460), 5′-CCTAG-3′ (part of Avr II site);

1776 bp (nucleotides 1461-3236) nos:npt II:3′nos sequence (complement of nucleotides 7483 to 9259 of pBin19, Gen Bank accession no. U09365);

9 bp (nucleotides 3237-3245) 5′-CCTAGGTAA-3′;

34 bp (nucleotides 3246-3279) Lox P sequence, 5′-ATAACTTCGTATAGCATAC ATTATACGAAGTTAT -3′ (SEQ ID NO:59);

3 bp (nucleotides 3280-3282) 5′-TAG-3′;

1848 bp (nucleotides 3283-5130) corresponding to nucleotides 2555 to 4402 of pBI101, Gen bank accession No. U12639, starting from the 5th bp of the ORF encoding 1805 bp. Upon linkage with the upstream TAG, it modifies the GUS ORF such that the initiation codon is missing, the ORF is extended at the 5′ end resulting in a 12-amino acid (ITSYSIHYTKLL; SEQ ID NO:66) N-terminal amino acid extension, and a changed 2^{nd} codon (from TTA to GTA) and 2^{nd} amino acid (from L to V) in the original GUS protein. Since the initiation Met is missing, this protein is not translatable;

22 bp (nucleotides 5131-5152) polylinker sequence, 5′-TGGGGAATTCCCCGG GGGTAC C-3′ (SEQ ID NO:60);

279 bp (nucleotides 5153-5431) 3′ region of nos (nucleotides 1824-2102 of nos gene, Genbank accession Nos. V00087, J01541); and

18 bp (nucleotides 5432-5449) polylinker sequence, 5′-GTCGACTCTAGAAA GCTT-3′ (SEQ ID NO:61).

Upon Cre-mediated site-specific recombination, the blocking fragment flanked by the Lox P sites is removed from pGV801 leaving behind a single Lox P site.

### Example 14

### Assay to Test Split Intein-Mediated Restoration of Cre Recombinase Activity Via Co-Bombardment in Tobacco Leaves

This Example describes the transformation of N and C-nucleotide sequences containing CreN- IntN and IntC-CreC and a trait expression construct containing GUS (from Examples 11 and 12) into tobacco leaves. When all three constructs were co-bombarded into the cells, positive GUS activity was observed.

Leaves of 2 month old wild type tobacco (var. Xanthi) plants were detached and placed on MS agar medium in petri dishes. Each leaf was bombarded with one of three DNA samples, with bombardment occurring in the following order:

| Order | Plasmid bombarded |
|---|---|
| 1. | 5 ug plasmid DNA without any GUS gene (‘dummy’ DNA) |
| 2. | 5 ug pGV801 reporter alone |
| 3. | 1 ug of pGV801 + pGV951 (35S: IntC--CreC: 3′nos) + pGV947 |
| | (35S: CreN-IntN: 3′nos) |

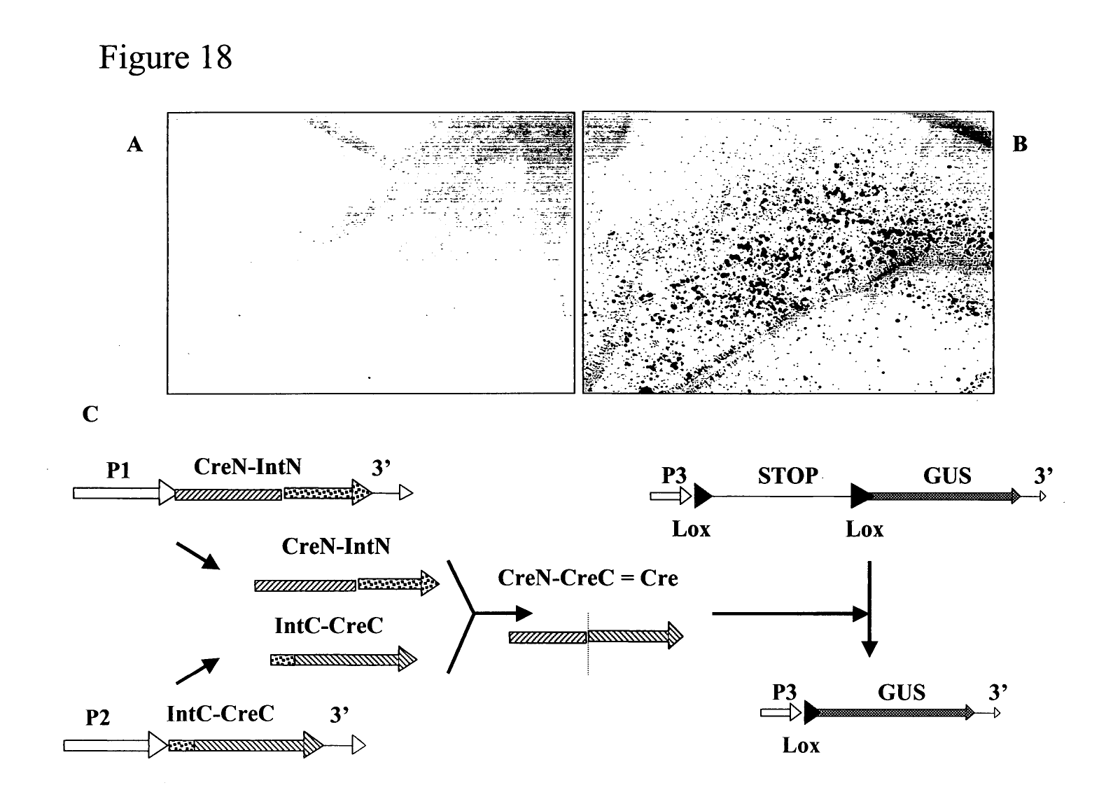

One day after bombardment the leaves were stained for GUS activity. FIG. 18A is a photograph of a GUS stained leaf bombarded with inactive reporter pGV801 alone. No GUS stain was observed with the ‘dummy’ DNA control (not shown) and with pGV801 alone (although, an occasional stained spot was seen that most likely represents homologous recombination between the Lox sites or contamination). In contrast, FIG. 18B is a photograph of a GUS stained leaf bombarded with the mixture of inactive reporter pGV801, pGV951, and pGV947. Significant positive GUS stained spots were observed in FIG. 18B. Specifically, GUS spots were seen only when pGV801 was co-bombarded with pGV951 and pGV947 in the manner of the positive control, i.e. pGV801 plus pNY102 (not shown).

The schematic shown in FIG. 18C graphically illustrates the molecular events that must occur for intein-mediated protein splicing of the Cre recombinase which thereby permits excision of the blocking fragment and expression of the GUS reporter. First, two different inactive recombinase elements are present within a cell (represented as P1-CreN-IntN and P2-IntC-CreC). Upon activation of the promoter (P1 and P2) within each construct (which can be constitutive or regulated), each recombinase element is transcribed and translated, producing an inactive protein precursor (CreN-IntN and IntC-CreC). When both protein precursors are simultaneously present within the cell, intein-mediated protein splicing occurs to excise each intein fragment and form a peptide bond between CreN and CreC, thus producing an active and functional Cre protein. With the expression of Cre, the blocking STOP fragment in the P3:Lox:STP:Lox:Gus construct is excised by site specific recombination, thereby allowing transcription and translation of the GUS transgene when the P3 promoter is activated.

### Example 15

### Construction of IntN Deletions

To test whether the full IntN sequence was required for activity, deletions were made in the 3′ end of the IntN encoding DNA in the 35S-Pro::GUSn/Intn::NOS-Ter test chimeric gene.

A DNA fragment containing 35S-Pro::GUSn/Intn::NOS-Ter and 35-Pro::Intc/GUSc::NOS-Ter (Example 4) was isolated from p35SGIN-35SIGC by SalI and Apal digestion, and cloned into pBS-SK(+) (Strategene, La Jolla, Calif.) between SalI and Apal sites resulting in plasmid pGIS. pGIS was digested with HindIII and XbaI and the 35-Pro::Intc/GUSc::NOS-Ter chimeric gene fragment was isolated and cloned into another pBS-SK(+) vector that had been digested with HindIII and XbaI. The remaining pGIS plasmid lacking 35S-Pro::GUSn/Intn::NOS-T was religated. Thus two pBS-SK(+)-based plasmids were constructed: pGISN containing the 35S-Pro::GUSn/Intn::NOS-T chimeric gene, and pGISC containing the 35-Pro::Intc/GUSc::NOS-Ter chimeric gene.

Plasmid pGISN was further subjected to deletion mutagenesis in the Intn coding sequence. For this purpose, the forward primer Nint-1 (SEQ ID NO:67, GTATCACCGTTTGTGTGMCA) and the reverse primers Nint-2 (SEQ ID NO:68, GGAATTCCTCA GATGTTCTCCAAAGTMGGAG), Nint-3 (SEQ ID NO:69, GGAATTCCTCMTCTGTAGTCAAGA ATCTATG), and Nint4 (SEQ ID NO:70, GGMTTCCTCAAACCTCTTGTTCACCCCTATC) were synthesized. Nint-1 matched a sequence within the GUS coding region, including a SnaBI site. Nint-2, Nint-3, and Nint-4 complemented the sequences upstream from the codons for IIe₁₀₀, Asp₇₆, and Val₅₅ within the Intn coding region, respectively. These reverse primers also had a common 5′ sequence (GGAATTCCTCA) to include an EcoRI site and a stop codon. The three primer pairs of Nint-1/Nint-2, Nint-1/Nint-3, and Nint-1/Nint-4 were used in PCR reactions (standard conditions using Strategy Pfu DNA polymerase ), with pGISN as a template. The three products were each digested with SnaBI and EcoRI, and cloned separately into pGISN that had been digested with SnaBI and EcoRI. In this manner the second half of the original GUSn-Intn fusion coding sequence was replaced with deletion fragments encoding Intn. The resulting three plasmids were named pGISND23, pGISND45, and pGISND68. The chimeric transgenes within these plasmids had structures similar to that in pGISN, but lacked the C-terminal 23, 45, and 68 codons of Intn coding sequence, respectively. The deletions were confirmed by DNA sequencing. The structures of these transgenes were summarized in FIG. 19A, and the sequences of each Intn deletion were detailed in FIG. 19B.

### Example 16

### Trans-Splicing Assay of IntN Deletions by Transient Transformation

The Intn deletions were assessed for their ability to support trans-splicing in soybean leaf tissue using the GUS marker in a transient expression assay.

Leaf tissue from 2-week old soybean plants was transformed by DNA bombardment as described in Example 11, except that 5 μg of each plasmid (for 2 plasmids, 10 μg total) was used to coat the gold particles and approximately one fifth of the DNA coated gold particles was bombarded per experiment. Four bombardments were conducted for each plasmid or plasmid combination. As in Example 11, the leaf tissue was assayed for GUS activity (Example 6) after two days of recovery.

First, each of the plasmids pGISC, pGISN, pGISND23, pGISND45, and pGISND68 was transformed individually. GUS staining showed that none of these transformations resulted in positive staining, indicating that neither partial GUS nor partial intein conferred β-glucuronidase activity. Next, pGISC was co-transformed with a second plasmid: either pGISN, pGISND23, pGISND45, or pGISND68. The assay was performed as above. Co-transformation of pGISC with pGISN, pGISND23, or pGISD45 resulted in strong GUS staining, indicating the presence of β-glucuronidase activity in the soybean leaf tissue. Thus the Intn sequences present in the 35S-Pro::GUSn/Intn::NOS-Tchimeric genes in the pGISN, pGISND23, and pGISD45 plasmids were able to support trans-splicing of the ExtN and ExtC portions of the GUS polypeptide.

However, when pGISC was co-bombarded with pGISD68, three of four transformed leaves showed no GUS staining, indicating that there was no β-glucuronidase activity at all in these bombarded leaves. One leaf bombarded with the pGISC and pGISND68 combination showed weak GUS staining. The blue color was very light and the spots occurred at much lower density, as compared to the GUS staining for pGICS co-transformed with pGISD45. Thus the Intn produced by pGISND68 having a deletion of 68 amino acid residues at its C-terminus, could not efficiently catalyze the splicing of the GUS polypeptide. However, Intn with a C-terminal deletion of 23 or 45 amino acids is able to catalyze efficient trans-splicing. The functional 78 amino acid Intn (45 amino acids eliminated) is given as SEQ ID NO:71 and its encoding nucleic acid sequence as SEQ ID NO:72.

What is claimed is:

1. An isolated polynucleotide comprising a nucleotide sequence that encodes a polypeptide comprising an ExtN, a ExtC, and an Int interposed between said ExtN and said ExtC, wherein:
said ExtN is the N-terminal portion of the polypeptide;
said Int is an intein; and
said ExtC is the C-terminal portion of the polypeptide,
and wherein at least a portion of said nucleotide sequence has been modified to contain plant optimized codons.

2. An isolated polynucleotide comprising a nucleotide sequence that encodes a fusion polypeptide consisting of an ExtN, a ExtC, and an Int interposed between said ExtN and said ExtC, wherein:
said ExtN is the N-terminal portion of the polypeptide;
said Int is an intein; and
said ExtC is the C-terminal portion of the polypeptide.

3. The polynucleotide of claim 1 or 2 wherein said Int is of bacterial origin.

4. The polynucleotide of claim 1 or 2 that further comprises a regulatory sequence.

5. The polynucleotide of claim 4 wherein said regulatory sequence is selected from the group consisting of a constitutive plant promoter, a plant tissue-specific promoter, and a plant developmental stage-specific promoter.

6. The polynucleotide of claim 1 or 2 wherein said Int is a naturally split intein consisting of an IntN and an IntC, wherein:
said IntN is the N-terminal portion of said naturally split intein; and
said IntC is the C-terminal portion of said naturally split intein.

7. The polynucleotide of claim 6 wherein said nucleotide sequence comprises:
an N-nucleotide sequence encoding said ExtN and said IntN; and
a C-nucleotide sequence encoding said IntC and said ExtC.

8. The polynucleotide of claim 7 that further comprises an N-regulatory sequence that is operably linked to said N-nucleotide sequence and a C-regulatory sequence that is operably linked to said C-nucleotide sequence, and wherein said C-regulatory sequence is interposed between said N-nucleotide sequence and said C-nucleotide sequence.

9. The polynucleotide of claim 6 wherein said IntN is encoded by the nucleotide sequence of SEQ ID NO:22.

10. The polynucleotide of claim 6 wherein said IntC is encoded by the nucleotide sequence of SEQ ID NO:24.

11. The polynucleotide of claim 6 wherein said IntN has the amino acid sequence of SEQ ID NO:23.

12. The polynucleotide of claim 6 wherein said IntC has the amino acid sequence of SEQ ID NO:25.

13. The polynucleotide of claim 1 or 2 wherein said ExtN and said ExtC together form an active protein.

14. A vector comprising the polynucleotide of claim 1 or 2.

15. A host cell comprising the polynucleotide of claim 1 or 2.

16. A transgenic plant comprising the polynucleotide of claim 1 or 2.

17. A seed comprising the polynucleotide of claim 1 or 2.

18. An isolated polynucleotide comprising a nucleotide sequence that encodes a polypeptide selected from the group consisting of:
an ExtN and an IntN; and
an ExtC and an IntC,
wherein said IntN and said IntC together form a naturally split intein.

19. A vector comprising the polynucleotide of claim 18.

20. A host cell comprising the polynucleotide of claim 18.

21. A transgenic plant comprising the polynucleotide of claim 18.

22. A seed comprising the polynucleotide of claim 18.

23. A method for producing a protein comprising an ExtN and a ExtC, said method comprising:
(a) obtaining an N-nucleotide sequence that encodes an N-polypeptide comprising an ExtN and an IntN;
(b) obtaining a C-nucleotide sequence that encodes a C-polypeptide comprising an IntC and an ExtC;
(c) transforming a plant host with said N-nucleotide sequence and said C-nucleotide sequence such that said plant produces said protein; and
(d) optionally recovering said protein.

24. The method of claim 23 wherein said (c) transforming comprises transforming said plant host with a vector that comprises said N-nucleotide sequence and said C-nucleotide sequence.

25. The method of claim 23 wherein said (c) transforming comprises separately transforming said plant host with said N-nucleotide sequence and said C-nucleotide sequence.

26. The method of claim 23 wherein at least a portion of at least one of said N-nucleotide sequence and said C-nucleotide sequence has been modified to contain plant optimized codons.

27. The method of claim 23 wherein said IntN and said IntC together form a naturally split intein.

28. The method of claim 23 wherein said IntN and said IntC together form an intein of bacterial origin.

29. The method of claim 23 wherein said plant host is a plant, a plant derived tissue, or a plant cell.

30. The method of claim 23 wherein said plant host is selected from food plants, non-food plants, arboreous plants, and aquatic plants.

31. The method of claim 23 wherein said protein consists of said ExtN and said ExtC.

32. The method of claim 31 wherein said protein is an active protein.

33. A method for producing a protein that comprises an ExtN and a ExtC, said method comprising:
(a) transforming an N-plant host with an N-polynucleotide comprising an N-nucleotide sequence that encodes an N-polypeptide comprising said ExtN and an IntN, such that said N-plant host produces said N-polypeptide;
(b) transforming a C-plant host with a C-polynucleotide comprising a C-nucleotide sequence that encodes a C-polypeptide comprising a IntC and said ExtC, such that said C-plant host produces said C-polypeptide; and
(c) crossing said N-plant host and said C-plant host to obtain a progeny of said N-plant host and said C-plant host, wherein said progeny comprises said protein.

34. The method of claim 33 wherein at least a portion of at least one of said N-nucleotide sequence and said C-nucleotide sequence has been modified to contain plant optimized codons.

35. The method of claim 33 wherein said IntN and said IntC form a naturally split intein.

36. The method of claim 33 wherein said IntN and said IntC together form an intein that is of bacterial origin.

37. The method of claim 33 wherein each of said N-plant host and said C-plant host is a plant, a plant derived tissue, or a plant cell.

38. The method of claim 33 wherein said plant host is selected from food plants, non-food plants, arboreous plants, and aquatic plants.

39. The method of claim 33 wherein said (a) transforming comprises introducing an N-vector into said N-plant host and wherein said N-vector comprises said N-nucleotide sequence, and wherein said (b) transforming comprises introducing a C-vector into said C-plant host and wherein said C-vector comprises said C-nucleotide sequence.

40. The method of claim 33 wherein said protein consists of said ExtN and said ExtC.

41. The method of claim 40 wherein said protein is an active protein.

42. A method for producing a protein comprising an ExtN and a ExtC, said method comprising:
(a) transforming an N-plant host with an N-polynucleotide comprising an N-nucleotide sequence that encodes an N-polypeptide comprising said ExtN and an IntN, such that said N-plant host produces said N-polypeptide;
(b) transforming a C-plant host with a C-polynucleotide comprising a C-nucleotide sequence that encodes a C-polypeptide comprising a IntC and said ExtC, such that said C-plant host produces said C-polypeptide;
(c) isolating said N-polypeptide from said N-plant host and said C-polypeptide from said C-plant host; and
(d) combining said N-polypeptide and said C-polypeptide in vitro to obtain said protein.

43. The method of claim 42 wherein at least a portion of at least one of said N-nucleotide sequence and said C-nucleotide sequence has been modified to contain plant optimized codons.

44. The method of claim 42 wherein said IntN and said IntC together form a naturally split intein.

45. The method of claim 42 wherein said IntN and said IntC together form an intein that is of bacterial origin.

46. The method of claim 42 wherein each of said N-plant host and said C-plant host is a plant, a plant derived tissue, or a plant cell.

47. The method of claim 42 wherein said plant host is selected from food plants, non-food plants, arboreous plants, and aquatic plants.

48. The method of claim 42 wherein said (a) transforming comprises introducing an N-vector into said N-plant host and wherein said N-vector comprises said N-nucleotide sequence, and wherein said (b) transforming comprises introducing a C-vector into said C-plant host, said C-vector comprising said C-nucleotide sequence.

49. The method of claim 48 wherein said protein consists of said ExtN and said ExtC.

50. The method of claim 49 wherein protein is an active protein.

51. A transgenic plant that produces an active protein comprising an ExtN and a ExtC, wherein said protein is produced from a polynucleotide comprising a nucleotide sequence that encodes said ExtN, said ExtC, and an intein interposed between said ExtN and said ExtC.

52. The plant of claim 51 wherein at least a portion of said nucleotide sequence has been modified to contain plant optimized codons.

53. The plant of claim 51 wherein said protein is expressed in at least one of a leaf, a root, a stem, a flower, a fruit, or a seed of the plant.

54. The plant of claim 51 that is selected from food plants, non-food plants, arboreous plants, and aquatic plants.

55. A transgenic plant that expresses a polypeptide selected from the group consisting of:
an ExtN and an IntN; and
an ExtC and an IntC,
wherein said IntN and said IntC together form an intein, and
wherein said ExtN and said ExtC together form an active protein.

56. The plant of claim 55 wherein said polypeptide is expressed in at least one of a leaf, a root, a stem, a flower, a fruit, or a seed of the plant.

57. The plant of claim 55 that is selected from food plants, non-food plants, arboreous plants, and aquatic plants.
